(19) United States
(12) Patent Application Publication
(10) Pub. No.: US 2026/0146266 A1
(43) Pub. Date: May 28, 2026

(54) RECOMBINANT HOST CELLS AND METHODS FOR THE PRODUCTION OF GLYCOLIC ACID

(71) Applicant: LYGOS, INC., Hayward, CA (US)

(72) Inventors: Jeffrey A. Dietrich, Hayward, CA (US); Mario Ouellet, Hayward, CA (US); Johan Van Walsem, Hayward, CA (US); Andrew Conley, Hayward, CA (US)

(21) Appl. No.: 18/684,261

(22) PCT Filed: Aug. 16, 2022
(86) PCT No.: PCT/US2022/040511

§ 371 Date: Feb. 16, 2024

Related U.S. Application Data
(60) Provisional application No. 63/233,494, filed on Aug. 16, 2021.

(51) Int. Cl. C12P 7/42; C12N 9/00; C12N 9/04; C12N 15/52

(52) U.S. Cl. CPC C12P 7/42; C12N 9/0006; C12N 9/93; C12N 15/52; C12Y 101/01037; C12Y 604/01001

(57) ABSTRACT

Methods and materials related to producing glycolic acid are disclosed. Specifically, isolated nucleic acids, polypeptides, host cells, methods and materials for producing glycolic acid by direct fermentation from sugars are disclosed.

### CROSS-REFERENCE TO RELATED APPLICATIONS

This application claims priority to and the benefit of U.S. Provisional Application Ser. No. 63/233,494 filed on Aug. 16, 2021 and entitled “RECOMBINANT HOST CELLS AND METHODS FOR THE PRODUCTION OF GLYCOLIC ACID,” the entire disclosure of which is incorporated herein by reference.

### FEDERALLY SPONSORED RESEARCH OR DEVELOPMENT

This invention was made with government support under Federal Gram No. DE-SC 0018759 awarded by the United States Department of Energy. The government has certain rights in the invention.

### SEQUENCE LISTING

The instant application contains a Sequence Listing which has been submitted via EFS-web and is hereby incorporated by reference in its entirety. The Sequence Listing, created on Aug. 15, 2022, is named 20220815_LYGOS-0017-04-WO_SequenceListing.txt, and is 377 KB in size.

### BACKGROUND

The long-term economic and environmental concerns associated with the petrochemical industry have provided the impetus for increased research, development, and commercialization of processes for conversion of carbon feedstocks into chemicals that can replace those petroleum feedstocks. One approach is the development of biorefining processes to convert renewable feedstocks into products that can replace petroleum-derived chemicals. Two common goals in improving a biorefining process include achieving a lower cost of production and reducing detrimental effects on the environment.

Glycolic acid (CAS No. 79-14-1) is an important precursor molecule that can be polymerized to produce the strong, malleable and biocompatible plastics polyglycolic acid (PGA) and poly[lactic-co-glycolic acid] (PLGA). These renewable and biodegradable plastics are in high demand in the food packaging, medical device and personal care industries.

Glycolic acid can be produced by several routes of chemical synthesis that arm each dependent on hazardous raw materials and extreme conditions during synthesis. Currently, glycolic acid may be prepared by: (1) hydrative carbonylation of formaldehyde with carbon monoxide and sulfuric acid (U.S. Pat. No. 2,152,852), or (2) saponification of chloroacetic acid with alkali metal hydroxide (U.S. Pat. No. 5,723,662). Both formaldehyde and chloroacetic acid are recognized as toxic air contaminants by the U.S. Environmental Protection Agency and the California Air Toxics Program (AB 1807 and AB 2728). Both methods require energy-intensive operating conditions that pose additional health and safety risks—carbonylation of formaldehyde requires run temperatures that range from 21 WC to 240° C. and a run pressure of around 900 atm while saponification of chloroacetic acid requires run temperatures that range from 100° C. to 160° C. Thus, there is a need for new low-cost, energy efficient, high yielding, and renewable manufacturing methods.

### SUMMARY

The present disclosure provides recombinant host cells and methods to produce glycolic acid by microbial fermentation from renewable feedstocks (e.g., glucose). Glycolic acid production according to the present disclosure utilizes an efficient carbon conversion route; in cases where glucose is used as the raw material and carbon dioxide is incorporated during product biosynthesis, the stoichiometric theoretical yield is 0.85 grams of glycolic acid for every gram of glucose, equating to one of the highest yielding products that can be manufactured microbially from glucose. Further, glycolic acid has two functional groups (i.e., an alcohol and a carboxylic acid) which make it a valuable chemical building block for a range of applications, including polymers and solvents.

The microbial fermentation process disclosed herein is run at both ambient atmospheric pressure and temperature, reducing the cost and environmental impact of manufacturing relative to the incumbent petrochemical processes. No organism in nature, including yeast, is known to produce glycolic acid from glucose in more than trace amounts. The materials and methods described herein comprise a renewable and cheaper starting material and an environmentally-benign biosynthetic process. The present disclosure provides a significant improvement to incumbent methods that comprise hazardous petrochemicals and extreme process conditions. The materials and methods described herein enable higher fermentation yields and productivities in the production of glycolic acid.

In a first aspect, the present disclosure provides recombinant host cells capable of producing glycolic acid, the host cells comprising one or more heterologous nucleic acids that encode the glycolic acid biosynthetic pathway, wherein the pathway enzymes comprise an oxaloacetate-forming enzyme, a malate dehydrogenase, a malate-CoA ligase, a malyl-CoA lyase, and a glyoxylate reductase. In some embodiments, the oxaloacetate-forming enzyme is pyruvate carboxylase, phosphoenolpyruvate carboxykinase, or phosphoenolpyruvate carboxylase. In some embodiments, the oxaloacetate-forming enzyme has at least 6(0% homology to SEQ ID NO: 57. In some embodiments, the oxaloacetate-forming enzyme is selected from the following: SEQ ID NO: 1, SEQ ID NO: 2, SEQ ID NO: 8, SEQ ID NO: 9, SEQ ID NO: 10, SEQ ID NO: 53, SEQ ID NO: 54, SEQ ID NO: 55, SEQ ID NO: 57, SEQ ID NO: 58, SEQ ID NO: 59, SEQ ID NO: 60, SEQ ID NO: 68, and SEQ ID NO: 69. In some embodiments, the malate dehydrogenase has at least 60% homology to SEQ ID NO: 4, SEQ ID NO: 64, SEQ ID NO: 70, or SEQ ID NO: 71, in some embodiments, the malate-CoA ligase has at least 60% homology to SEQ ID NO: 6 and SEQ ID NO: 7, SEQ ID NO: 11 and SEQ ID NO: 12, SEQ ID NO: 13 and SEQ ID NO: 14, SEQ ID NO: 15 and SEQ ID NO: 16, SEQ ID NO: 63 and SEQ ID NO: 3, or SEQ ID NO: 56 and SEQ ID NO: 52. In some embodiments, the malyl-CoA lyase has at least 40% homology to SEQ ID NO: 21. In some embodiments, the malyl-CoA lyase is selected from the following: SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30.

As noted in detail in the definitions provided below, proteins that share a specific function are not always defined or limited by percent sequence homology. In some cases, a protein with low percent sequence homology with a reference protein is able to carry out the identical biochemical reaction as the reference protein. Such proteins may share three-dimensional structure which enables shared specific functionality, but not necessarily sequence similarity. Such proteins may share an insufficient amount of sequence similarity to indicate that they are homologous via evolution from a common ancestor and would not be identified by a BLAST search or other sequence-based searches. Thus, in some embodiments of the present disclosure, homologous proteins comprise proteins that lack substantial sequence similarity but share substantial functional similarity and/or substantial structural similarity.

In some embodiments, the glyoxylate reductase has at least 60% homology to SEQ ID NO: 22. In some embodiments, the glyoxylate reductase is selected from the following: SEQ ID NO: 5, SEQ ID NO: 17, SEQ ID NO: 18, SEQ ID NO: 19, SEQ ID NO: 20, SEQ ID NO: 31, SEQ ID NO: 32, SEQ ID NO: 33. SEQ ID NO: 34, SEQ ID NO: 66, SEQ ID NO: 80, SEQ ID NO: 81, SEQ ID NO: 82, SEQ ID NO: and 83 SEQ ID NO: 84.

In a second aspect, the recombinant host cell is a yeast cell. In certain embodiments, the yeast cell belongs to the Issatchenkia orientalis/Pichia fermentans clade. In some embodiments, the yeast cell belongs to the genus Pichia, Issatchenkia or Candida. In some embodiments, the yeast cell is Pichia kudriavzevii. In some embodiments, the yeast cell belongs to the Saccharomyces clade. In some embodiments, the yeast cell is Saccharomyces cerevisiae. In other embodiments, the recombinant host cell is a prokaryotic cell. In some embodiments, the prokaryotic cell belongs to the genus Escherichia, Corynebacterium, Bacillus, or Lactacoccus. In some embodiments, the prokaryotic cell is Escherichia coli, Corynebacterium glutamicum, Bacillus subtilis, or Lactococcus lactis.

In a third aspect, the present disclosure provides recombinant host cells that further comprise one or more heterologous nucleic acids encoding one or more ancillary proteins that function in redox cofactor recycling, redox cofactor biogenesis, organic acid transport, carbon fixation, or improving pathway flux through the glycolic acid pathway. In some embodiments, the one or more ancillary proteins is a glycolic acid transporter, a carbon fixation enzyme, or a combination thereof. In some embodiments, the glycolic acid transporter has at least 60% homology to SEQ ID NO: 35. SEQ ID NO: 36, SEQ ID NO: 37, SEQ ID NO: 38, SEQ ID NO: 65. SEQ ID NO: 67, SEQ ID NO: 76, SEQ ID NO: 77, SEQ ID NO: 78, or SEQ ID NO: 79. In some embodiments, the glycolic acid transporter is selected from the following: SEQ ID NO: 35, SEQ ID NO: 36, SEQ ID NO: 37, SEQ ID NO: 38, SEQ ID NO: 65, SEQ ID NO: 67, SEQ ID NO: 76, SEQ ID NO: 77. SEQ ID NO: 78, and SEQ ID NO: 79. In some embodiments, the carbon fixation enzyme has at least 60% homology to SEQ ID NO: 44, SEQ ID NO: 45, SEQ ID NO: 46, SEQ ID NO: 47, SEQ ID NO: 48, SEQ ID NO: 49, SEQ ID NO: 50, or SEQ ID NO: 51. In some embodiments, the carbonic anhydrase is selected from the following: SEQ ID NO: 44, SEQ ID NO: 45. SEQ ID NO: 46. SEQ ID NO: 47, SEQ ID NO: 48, SEQ ID NO: 49. SEQ ID NO: 50, and SEQ ID NO: 51.

In a fourth aspect, the present disclosure provides recombinant host cells that further comprise a genetic disruption of one or more genes, wherein the one or more genes encodes pyruvate decarboxylase, pyruvate dehydrogenase complex, glycerol-3-phosphate dehydrogenase, malate synthase, glycine dehydrogenase, or a combination thereof. In some embodiments, the one or more genes has at least 60% amino acid homology to SEQ ID NO: 39, SEQ ID NO: 40, SEQ ID NO: 41, SEQ ID NO: 42. SEQ ID NO: 43, SEQ ID NO: 72, SEQ ID NO: 73, SEQ ID NO: 74, or SEQ ID NO: 75. In some of these embodiments, the recombinant host cells produce less than 5 g/l ethanol. In some of these embodiments, the recombinant host cells produce less than 10 g/l glycerol. In some of these embodiments, the recombinant host cells produce less than 10 g/l malate.

In a fifth aspect, the present disclosure provides a method for the production of glycolic acid that comprises culturing the recombinant host cells provided by the present disclosure for a sufficient period of time to produce glycolic acid. In some embodiments, the method further comprises an oxygen transfer rate that is greater than 10 mmol/l/hr. In some embodiments, the method further comprises a culturing temperature of 25° C. to 45° C. In some embodiments, the method further comprises a final fermentation pH of between pH 4 and pH 8. In some embodiments, the method further comprises a final fermentation pH of less than about pH 5. In some embodiments, the method further comprises carbon dioxide supplementation. In some embodiments, the method further comprises bicarbonate supplementation. In some embodiments, the method further comprises producing a solution containing at least 50 g/l glycolic acid. In some embodiments, the method further comprises providing at least 100 g/l glucose to the recombinant host cells and converting at least 25% (w/w) of said glucose to glycolic acid (i.e., a 25% yield).

### BRIEF DESCRIPTION OF THE DRAWINGS

Those skilled in the art will understand that the drawings described herein are for illustration purposes only. The drawings are not intended to limit the scope of the present disclosure.

FIG. 1. The glycolic acid pathway provided by the present disclosure converts 1 molecule of glucose and 2 molecules of CO₂ to 2 molecules of glycolic acid and 2 molecules of acetyl-CoA.

### DETAILED DESCRIPTION

The present disclosure provides recombinant host cells, materials and methods for the biological production and purification of glycolic acid.

While the present disclosure describes aspects and specific embodiments, those skilled in the an will recognize that various changes may be made and equivalents may be substituted without departing therefrom. The present disclosure is not limited to particular nucleic acids, expression vectors, enzymes, biosynthetic pathways, host microorganisms, or processes, as such may vary. The terminology used herein is for the purposes of describing particular aspects and embodiments only, and is not to be construed as limiting. In addition, many modifications may be made to adapt a particular situation, material, composition of matter, process, process steps or process flows, in accordance with the present disclosure. All such modifications are within the scope of the claims appended hereto.

### SECTION 1: DEFINITIONS

In this specification and in the claims that follow, reference will be made to a number of terms that shall be defined to have the following meanings.

The term “accession number” and similar terms such as “protein accession number”, “UniProt ID”, “gene ID” and “gene accession number” refer to designations given to specific proteins or genes. These identifiers described a gene or protein sequence in publicly accessible databases such as the National Center for Biotechnology Information (NCBI).

The term “heterologous” as used herein refers to a material that is non-native to a cell. For example, a nucleic acid is heterologous to a cell, and so is a “heterologous nucleic acid” with respect to that cell if at least one of the following is true: 1) the nucleic acid is not naturally found in that cell (that is, it is an “exogenous” nucleic acid); 2) the nucleic acid is naturally found in a given host cell (that is, “endogenous to”), but the nucleic acid or the RNA or protein resulting from transcription and translation of this nucleic acid is produced or present in the host cell in an unnatural (e.g., greater or lesser than naturally present) amount; 3) the nucleic acid comprises a nucleotide sequence that encodes a protein endogenous to a host cell, but the nucleic acid sequence differs in sequence from the endogenous nucleotide sequence that encodes that same protein (having the same or substantially the same amino acid sequence), typically resulting in the protein being produced in a greater amount in the cell, or in the case of an enzyme, producing a mutant version possessing altered (e.g., higher or lower or different) activity; and/or 4) the nucleic acid comprises two or more nucleotide sequences that are not found in the same relationship to each other in the cell. As another example, a protein is heterologous to a host cell if it is produced by translation of RNA or the corresponding RNA is produced by transcription of a heterologous nucleic acid. Further, a protein is also heterologous to a host cell if it is a mutated version of an endogenous protein, and the mutation was introduced by genetic engineering.

The term “homologous”, as well as variations thereof, such as “homology”, refers to the similarity of a nucleic acid or amino acid sequence, typically in the context of a coding sequence for a gene or the amino acid sequence of a protein. Homology searches can be employed using a known amino acid or coding sequence (the “reference sequence”) for a useful protein to identify homologous coding sequences or proteins that have similar sequences and thus are likely to perform the same useful function as the protein defined by the reference sequence. As will be appreciated by those of skill in the art, a protein having homology to a reference protein can be determined, for example and without limitation, by a BLAST (https://blast.ncbi.alm.nih.gov) search. A protein with high percent homology is highly likely to cany out the identical biochemical reaction as the reference protein. In some cases, two enzymes having greater than 60% homology will carry out identical biochemical reactions, and the higher the homology. i.e., 60%, 70%, 80%, 90.% or greater than 95% homology, the more likely the two proteins have the same or similar function. A protein with at least 60% homology to its reference protein is defined as substantially homologous. Any protein substantially homologous to a reference sequence can be used in a host cell according to the present disclosure.

Generally, homologous proteins share substantial sequence homology. Sets of homologous proteins generally possess one or more specific amino acids that are conserved across all members of the consensus sequence protein class. The percent sequence homology of a protein relative to a consensus sequence is determined by aligning the protein sequence against the consensus sequence. Practitioners in the art will recognized that various sequence alignment algorithms are suitable for aligning a protein with a consensus sequence. See, for example, Needleman, S B, et al., “A general method applicable to the search for similarities in the amino acid sequence of two proteins.” Journal of Molecular Biology 48 (3): 443-53 (1970). Following alignment of the protein sequence relative to the consensus sequence, the percentage of positions where the protein possesses an amino acid described by the same position in the consensus sequence determines the percent sequence homology. When a degenerate amino acid is present (i.e., B, Z, X, J or “+”) in a consensus sequence, any of the amino acids described by the degenerate amino acid may be present in the protein at the aligned position for the protein to be homologous to the consensus sequence at the aligned position. When it is not possible to distinguish between two closely related amino acids, the following one-letter symbol is used—“B” refers to aspartic acid or asparagine; “Z” refers to glutamine or glutamic acid; “J” refers to leucine or isoleucine; and “X” or “+” refers to any amino acid.

A dash (−) in a consensus sequence indicates that there is no amino acid at the specified position. A plus (+) in a consensus sequence indicates any amino acid may be present at the specified position. Thus, a plus in a consensus sequence herein indicates a position at which the amino acid is generally non-conserved; a homologous enzyme sequence, when aligned with the consensus sequence, can have any amino acid at the indicated “+” position.

In addition to identification of useful enzymes by percent sequence homology with a given consensus sequence, enzymes useful in the compositions and methods provided herein can also be identified by the occurrence of highly conserved amino acid residues in the query protein sequence relative to a consensus sequence. For each consensus sequence provided herein, a number of highly conserved amino acid residues are described. Enzymes useful in the compositions and methods provided herein include those that comprise a substantial number, and sometimes all, of the highly conserved amino acids at positions aligning with the indicated residues in the consensus sequence. Those skilled in the art will recognize that, as with percent homology, the presence or absence of these highly conserved amino acids in a query protein sequence can be determined following alignment of the query protein sequence relative to a given consensus sequence and comparing the amino acid found in the query protein sequence that aligns with each highly conserved amino acid specified in the consensus sequence.

Proteins that share a specific function are not always defined or limited by percent sequence homology. In some cases, a protein with low percent sequence homology with a reference protein is able to carry out the identical biochemical reaction as the reference protein. Such proteins may share three-dimensional structure which enables shared specific functionality, but not necessarily sequence similarity. Such proteins may share an insufficient amount of sequence similarity to indicate that they are homologous via evolution from a common ancestor and would not be identified by a BLAST search or other sequence-based searches. Thus. In some embodiments of the present disclosure, homologous proteins comprise proteins that lack substantial sequence similarity but share substantial functional similarity and/or substantial structural similarity.

As used herein, the term “express”, when used in connection with a nucleic acid encoding an enzyme or an enzyme itself in a cell, means that the enzyme, which may be an endogenous or exogenous (heterologous) enzyme, is produced in the cell. The term “overexpress”, in these contexts, means that the enzyme is produced at a higher level. i.e., enzyme levels are increased, as compared to the wild-type, in the case of an endogenous enzyme. Those skilled in the art appreciate that overexpression of an enzyme can be achieved by increasing the strength or changing the type of the promoter used to drive expression of a coding sequence, increasing the strength of the ribosome binding site or Kozak sequence, increasing the stability of the mRNA transcript, altering the codon usage, increasing the stability of the enzyme, and the like.

The terms “expression vector” or “vector” refer to a nucleic acid and/or a composition comprising a nucleic acid that can be introduced into a host cell, e.g., by transduction, transformation, or infection, such that the cell then produces (i.e., expresses) nucleic acids and/or proteins other than those native to the cell, or in a manner not native to the cell, that are contained in or encoded by the nucleic acid so introduced. Thus, an “expression vector” contains nucleic acids (ordinarily DNA) to be expressed by the host cell. Optionally, the expression vector can be contained in materials to aid in achieving entry of the nucleic acids into the host cell, such as the materials associated with a virus, liposome, protein coating, or the like. Expression vectors suitable for use in various aspects and embodiments of the present disclosure include those into which a nucleic acid sequence can be, or has been, inserted, along with any preferred or required operational elements. Thus, an expression vector can be transferred into a host cell and, typically, replicated therein (although, one can also employ. In some embodiments, non-replicable vectors that provide for “transient” expression). In some embodiments, an expression vector that integrates into chromosomal, mitochondrial, or plastid DNA is employed. In other embodiments, an expression vector that replicates extrachromosomally is employed. Typical expression vectors include plasmids, and expression vectors typically contain the operational elements required for transcription of a nucleic acid in the vector. Such plasmids, as well as other expression vectors, are described herein or are well known to those of ordinary skill in the art.

The terms “ferment”. “fermentative”, and “fermentation” are used herein to describe culturing microbes under conditions to produce useful chemicals, including but not limited to conditions under which microbial growth, be it aerobic or anaerobic, occurs.

The terms “recombinant host cell”, “recombinant host microorganism”, and “strain” are used interchangeably herein to refer to a living cell that can be (or has been) transformed via insertion of an expression vector. A host cell or microorganism as described herein may be a prokaryotic cell (e.g., a microorganism of the kingdom Eubacteria) or a eukaryotic cell. As will be appreciated by one of skill in the art, a prokaryotic cell lacks a membrane-bound nucleus, while a eukaryotic cell has a membrane-bound nucleus.

The terms “isolated” or “pure” refer to material that is substantially, e.g., greater than 50% or greater than 75%, or essentially, e.g., greater than 90%, 95%, 98% or 99%, free of components that normally accompany it in its native state, e.g., the state in which it is naturally found or the state in which it exists when it is first produced. Additionally, any reference to a “purified” material is intended to refer to an isolated or pure material.

As used herein, the term “nucleic acid” and variations thereof shall be generic to polydeoxyribonucleotides (containing 2-deoxy-D-ribose, polyribonucleotides (containing D-ribose), segments of polydeoxyribonucleotides, and segments of polyribonucleotides. “Nucleic acid” can also refer to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. As used herein, the symbols for nucleotides and polynucleotides are those recommended by the IUPAC-IUB Commission of Biochemical Nomenclature (Biochem. 9:4022, 1970). A “nucleic acid” may also be referred to herein with respect to its sequence, the order in which different nucleotides occur in the nucleic acid, as the sequence of nucleotides in a nucleic acid typically defines its biological activity, e.g., as in the sequence of a coding region, the nucleic acid in a gene composed of a promoter and coding region, which encodes the product of a gene, which may be an RNA, e.g., a rRNA, tRNA, or mRNA, or a protein (where a gene encodes a protein, both the mRNA and the protein are “gene products” of that gene).

In the present disclosure, the term “genetic disruption” refers to several ways of altering genomic, chromosomal or plasmid-based gene expression. Non-limiting examples of genetic disruptions include CRISPR. RNAi, nucleic acid deletions, nucleic acid insertions, nucleic acid substitutions, nucleic acid mutations, knockouts, premature stop codons and transcriptional promoter modifications. In the present disclosure “genetic disruption” is used interchangeably with “genetic modification”, “genetic mutation” and “genetic alteration,” Genetic disruptions give rise to altered gene expression and or altered protein activity. Altered gene expression encompasses decreased, eliminated and increased gene expression levels. In some examples, gene expression results in protein expression, in which case the term “gene expression” is synonymous with “protein expression”.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used herein, “recombinant” refers to the alteration of genetic material by human intervention. Typically, recombinant refers to the manipulation of DNA or RNA in a cell or virus or expression vector by molecular biology (recombinant DNA technology) methods, including cloning and recombination. Recombinant can also refer to manipulation of DNA or RNA in a cell or virus by random or directed mutagenesis. A “recombinant” cell or nucleic acid can typically be described with reference to how it differs from a naturally occurring counterpart (the “wild-type”). In addition, any reference to a cell or nucleic acid that has been “engineered” or “modified” and variations of those terms, is intended to refer to a recombinant cell or nucleic acid.

The terms “transduce”, “transform”, “transfect” and variations thereof as used herein refer to the introduction of one or more nucleic acids into a cell. For practical purposes, the nucleic acid must be stably maintained or replicated by the cell for a sufficient period of time to enable the function(s) or product(s) it encodes to be expressed for the cell to be referred to as “transduced”, “transformed”, or “transfected”. As will be appreciated by those of skill in the art, stable maintenance or replication of a nucleic acid may take place either by incorporation of the sequence of nucleic acids into the cellular chromosomal DNA, e.g., the genome, as occurs by chromosomal integration, or by replication extrachromosomally, as occurs with a freely-replicating plasmid. A virus can be stably maintained or replicated when it is “infective”: when it transduces a host microorganism, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

As used herein, “glycolic acid” is intended to mean the molecule having the chemical formula C₂H₄O₃ and a molecular mass of 76.05 g/mol (CAS No. 79-14-1). The terms “glycolic acid”, “hydroxyacetic acid”, and “2-hydroxyacetic acid” are used interchangeably in the present disclosure, and practitioners skilled in the art understand that these terms are synonyms.

In conditions with pH values higher than the pKa of glycolic acid (e.g., about pH>3.83 when using a sodium base, such as sodium hydroxide), glycolic acid is deprotonated to the glycolate/glycollate anion C₂H₃O₃⁺. Herein, “glycolate” is also used interchangeably with “glycollate”, “glycolate anion,” “glycollate anion”, “hydroxyacetate”, and “2-hydroxyacetate”, and practitioners skilled in the art understand that these terms are synonyms.

Further, the glycolate anion is capable of forming an ionic bond with a cation to produce a glycolate salt. The term “glycolate” is intended to mean a variety of glycolate salt forms, and is used interchangeably with “glycolate salt”. Non-limiting examples of glycolates comprise sodium glycolate (CAS No. 2836-32-0), calcium glycolate (CAS No. 996-23-6), potassium glycolate (CAS No. 25904-89-6), ethyl glycolate (CAS No. 623-50-7), and methyl glycolate (CAS No, 96-35-5).

In conditions with pH values lower than the pKa of glycolate acid (e.g., pH<3.83), the glycolate anion is protonated to form glycolic acid. Herein, “glycolate” is also used interchangeably with “glycolic acid” and practitioners in the art understand that these terms are synonyms.

As used herein, “glycolate ester” is intended to mean an ester derived from glycolic acid, and practitioners in the art understand that it is synonymous with “alkyl glycolate”. Non-limiting examples of glycolate esters comprise ethyl glycolate (CAS No. 623-50-7), methyl glycolate (CAS No. 96-35-3) and benzyl glycolate (CAS No. 30379-58-9).

The glycolic acid, glycolate salts and glycolate esters of the present disclosure are synthesized from biologically produced organic components by a fermenting microorganism. For example, glycolic acid, glycolate salts, glycolate esters, or their precursor(s) are synthesized from the fermentation of sugars by recombinant host cells of the present disclosure. Practitioners skilled in the art understand that the prefix “bio-” or the adjective “bio-based” may be used to distinguish these biologically-produced glycolic acid and glycolates from those that are derived from petroleum feedstocks. As used herein, “glycolic acid” is defined as “bio-based glycolic acid”, “glycolate salt” is defined as “bio-based glycolate salt”, and “glycolate ester” is defined as “bio-based glycolate ester”.

The term “byproduct” or “by-product” means an undesired product related to the production of a target molecule. In the present disclosure, “byproduct” is intended to mean any amino acid, amino acid precursor, chemical, chemical precursor, organic acid, organic acid precursor, ester, ester precursor, biofuel, biofuel precursor, metabolite, or small molecule, that may accumulate during biosynthesis or chemical synthesis of glycolic acid, glycolate, glycolate ester, or other downstream product of the present disclosure, in some cases, “byproduct” accumulation may decrease the yields, titers or productivities of the target product (i.e., glycolic acid, glycolate, glycolate ester, or other downstream product) in a fermentation or in synthesis.

Malic acid is a pathway intermediate of the glycolic acid biosynthetic pathway of the present disclosure. As used herein, “malic acid” is intended to mean the molecule having the chemical formula C₄H₆O₅ and a molecular mass of 134.09 g/mol (CAS No. 6915-15-7). The terms “malic acid”, “hydroxybutanedioic acid”, and “DL-malic acid” are used interchangeably in the present disclosure, and practitioners skilled in the art understand that these terms are synonyms.

In conditions with pH values higher than about 5.03, malic acid is deprotonated to the malate anion C₄H₄O₅⁻². Herein, “malate anion” is also used interchangeably with “malate” and practitioners skilled in the art understand that these terms are synonyms.

Further, the malate anion is capable of forming an ionic bond with a cation to produce a malate salt. The term “malate” is intended to mean a variety of malate salt forms and is used interchangeably with “malate salt”. Non-limiting examples of malates comprise sodium malate (CAS No. 676-46-0), calcium malate (CAS No. 17482-42-7), potassium malate (CAS No. 585-09-1), and ammonium malate (CAS No. 6283-27-8)

In conditions with pH values lower than 3.4, the malate anion is protonated to form malic acid. Herein, “malate” is also used interchangeably with “malic acid” and practitioners in the an understand that these terms are synonyms.

Malic acid exists in 2 stereoisomeric forms (also known as optical isomers)-D- and L-enantiomers; hence the synonym DL-malic acid is sometimes used for malic acid. D- and L-enantiomers of malic acid are molecules that share the same molecular weight of 134.09 g/mol and are non-superimposable mirror images of each other, analogous to one's left and right hands being the same and not superimposable by simple reorientation around an axis. Separately, enantiomers have identical chemical and physical properties (except when another enantiomer is present), but the D- or (+)-enantiomer rotates polarized light clockwise (to the right) while the L- or (−) enantiomer rotates polarized light counterclockwise (to the left).

The redox cofactor nicotinamide adenine dinucleotide. NAD, comes in 2 forms—phosphorylated and un-phosphorylated. The term “NAD(P)” refers to both phosphorylated (NADP) and un-phosphorylated (NAD) forms, and encompasses oxidized versions (NAD⁺ and NADP⁺) and reduced versions (NADH and NADPH) of both forms. The term “NAD(P)⁺” refers to the oxidized versions of phosphorylated and un-phosphorylated NAD, i.e., NAD⁺ and NADP. Similarly, the term “NAD(P)H” refers to the reduced versions of phosphorylated and un-phosphorylated NAD, i.e., NADH and NADPH. When NAD(P)H is used to describe the redox cofactor in an enzyme catalyzed reaction, it indicates that NADH and/or NADPH is used. Similarly, when NAD(P)⁺ is the notation used, it indicates that NAD⁺ and/or NADP⁺ is used. Those skilled in the art will also appreciate that while many proteins may only bind either a phosphorylated or un-phosphorylated cofactor, there are redox cofactor promiscuous proteins, natural or engineered, that are indiscriminate; in these cases, the protein may use either NADH and/or NADPH. In some embodiments, enzymes that preferentially utilize either NAD(P) or NAD may carry out the same catalytic reaction when bound to either form.

Various values for temperatures, titers, yields, oxygen uptake rate (OUR), and pH are recited in the description and in the claims. It should be understood that these values are not intended to convey that an exact temperature is or was critical to function of the present disclosure, unless that is explicitly stated. For example, a temperature range of from about 30° C. to about 42° C. in the context of a fermentation conveys to the artisan of ordinary skill that this is a preferred range, and that varying somewhat from it, whether at the high or low end, by a few degrees, may affect efficiency or yield but should still be operative. e.g., fermentations in the range 25° C. to 44° C. would be understood to be possible and, if conducted in accordance with the present disclosure, would still be operable as disclosed. It should be understood that numerical ranges recited include the recited minimum value and the recited maximum value.

### SECTION 2: RECOMBINANT HOST CELLS FOR PRODUCTION OF GLYCOLIC ACID

### 2.1 Host Cells

The present disclosure provides recombinant host cells engineered to produce glycolic acid, wherein the recombinant host cells comprise one or more heterologous nucleic acids encoding one or more glycolic acid pathway enzymes. In certain embodiments, the recombinant host cells further comprise one or more heterologous nucleic acids encoding one or more ancillary gene products (i.e., gene products other than the glycolic acid pathway enzymes) that improve yields, titers and/or productivities of glycolic acid. In particular embodiments, the recombinant host cells further comprise disruptions or deletions of endogenous nucleic acids that improve yields, titers and/or productivities of glycolic acid. In some embodiments, the recombinant host cells are capable of producing glycolic acid under aerobic conditions. In some embodiments, the recombinant host cells are capable of producing glycolic acid under substantially anerobic conditions. The recombinant host cells produce glycolic acid at increased titers, yields and productivities as compared to a parental host cell that does not comprise said heterologous nucleic acids.

In some embodiments, the recombinant host cells further comprise one or more heterologous nucleic acids encoding one or more ancillary gene products (i.e., gene products other than the downstream product pathway enzymes) that improve yields, titers and/or productivities of glycolic acid. In particular embodiments, the recombinant host cells further comprise disruptions or deletions of endogenous nucleic acids that improve yields, titers and/or productivities of glycolic acid. In some embodiments, the recombinant host cells are capable of producing glycolic acid under aerobic conditions. In some embodiments, the recombinant host cells are capable of producing glycolic acid under substantially anaerobic conditions.

Any suitable host cell may be used in practice of the methods of the present disclosure, and exemplary host cells useful in the compositions and methods provided herein include archaeal, prokaryotic, or eukaryotic cells. In an embodiment of the present disclosure, the recombinant host cell is a prokaryotic cell. In an embodiment of the present disclosure, the recombinant host cell is a eukaryotic cell. In an embodiment of the present disclosure, the recombinant host cell is a Pichia kudriavzevii (P. kudriavzevii) strain. Methods of construction and genotypes of these recombinant host cells are described herein.

### 2.1.1 Yeast Cells

In an embodiment of the present disclosure, the recombinant host cell is a yeast cell. Yeast cells are excellent host cells for construction of recombinant metabolic pathways comprising heterologous enzymes catalyzing production of small-molecule products. There are established molecular biology techniques and nucleic acids encoding genetic elements necessary for construction of yeast expression vectors, including, but not limited to, promoters, origins of replication, antibiotic resistance markers, auxotrophic markers, terminators, and the like. Additionally, techniques for integration/insertion of nucleic acids into the yeast chromosome by homologous recombination are well established. Yeast also offers a number of advantages as an industrial fermentation host. Yeast cells can generally tolerate high concentrations of organic acids and maintain cell viability at low pH, can grow under both aerobic and anaerobic culture conditions, and there are established fermentation broths and fermentation protocols. This characteristic results in efficient product biosynthesis when the host cell is supplied with a carbohydrate carbon source.

In various embodiments, yeast cells useful in the methods of the present disclosure include yeasts of the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuratshia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosportion, Schizosaccharomyces, Schwanniomyces, Sporidiobolus, Sporobolomyces, Sporopachydermia, Stephamoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In various embodiments, the yeast cell is of a species selected from the non-limiting group comprising Candida albicans, Candida ethanolica, Candida guilliermondii, Candida krusei, Candida lipolytica, Candida methanosorbosa, Candida sonorensis, Candida tropicalis, Candida utilis, Cryptococcus curvatus, Hansenula polymorpha, Issatchenkia orientalis, Kluyveromyces lactis, Kluyveromyces marxianus, Kluyveromyces thermotolerans, Komagataella pastoris, Lipomyces starkeyi, Pichia angusta, Pichia deserticola, Pichia galeiformis, Pichia kodamae, Pichia kudriavzevii (P. kudriavzevii), Pichia membranaefaciens, Pichia methanolica, Pichia pastoris, Pichia salicaria, Pichia stipitis, Pichia thermotolerans, Pichia trehalophila, Rhodosporidium toruloides, Rhodotorula glutinis, Rhodotorula graminis, Saccharomyces bayanus, Saccharomyces boulardi, Saccharomyces cerevisiae (S. cerevisiae), Saccharomyces kluyveri, Schizosaccharomyces pombe (S. pombe) and Yarrowia lipolytica. One skilled in the art will recognize that this list encompasses yeast in the broadest sense.

The Crabtree phenomenon refers to the capability of yeast cells to convert glucose to alcohol in the presence of high sugar concentrations and oxygen instead of producing biomass via the tricarboxylic acid (TCA)cycle. Yeast cells produce alcohol to prevent growth of competing microorganisms in high sugar environments, which yeast cells can utilize later on when the sugars are depleted. One skilled in the art will recognize that many yeast can typically use 2 pathways to produce ATP from sugars: the first involves the conversion of a sugars (via pyruvate) to carbon dioxide via the TCA cycle, and the second involves the conversion of sugars (via pyruvate) to ethanol. Yeast cells that display a Crabtree effect (known as Crabtree-positive yeast cells) are able to simultaneously use both pathways. Yeast cells that do not display a Crabtree effect (known as Crabtree-negative yeast cells) primarily convert pyruvate to ethanol when oxygen is absent. In some embodiments of the present disclosure, the host cell is a Crabtree-positive yeast cell. In other embodiments, the host cell is a Crabtree-negative yeast cell. In certain embodiments, the host cell displays a phenotype along a continuum of traits between Crabtree-positive and Crabtree-negative and is thus neither exclusively a Crabtree-positive yeast cell nor Crabtree negative yeast cell. In certain embodiments, it is advantageous to use a Crabtree-negative yeast or a yeast with perceptible Crabtree-negative tendencies or traits to produce glycolic acid because high glucose concentrations can be maintained during product biosynthesis without ethanol accumulation; ethanol is an undesired byproduct in glycolic acid production. P. kudriavzevii does not produce appreciable amounts of ethanol from pyruvate at high glucose concentrations in the presence of oxygen, and as such is a Crabtree-negative yeast. In some embodiments, the host cell is P. kudriavzevii.

In certain embodiments, the recombinant yeast cells provided herein are engineered by the introduction of one or more genetic modifications (including, for example, heterologous nucleic acids encoding enzymes and/or the disruption of native nucleic acids encoding enzymes) into a Crabtree-negative yeast cell. In certain of these embodiments, the host cell belongs to the Pichia/Issatchenkia/Saturnospora/Dekkera clade, in certain of these embodiments, the host cell belongs to the genus selected from the group comprising Pichia, Issatchenkia, or Candida. In certain embodiments, the host cell belongs to the genus Pichia, and in some of these embodiments the host cell is P. kudriavzevii. Members of the Pichia/Issatchenkia/Saturnospora/Dekkera or the Saccharomyces clade are identified by analysis of their 26S ribosomal DNA using the methods described by Kurtzman C. P., and Robnett C. J., (“Identification and Phylogeny of Ascomycetous Yeasts from Analysis of 23 Nuclear Large Subunit (26S) Ribosomal DNA Partial Sequences”. Atonie van Leeuwenhoek 73(4):331-371; 1998), Kurtzman and Robnett report analysis of approximately 500 ascomycetous yeasts, which were analyzed for the extent of divergence in the variable D1/D2 domain of the large subunit (26S) ribosomal DNA. Host cells encompassed by a clade exhibit greater sequence identity in the D1/D2 domain of the 26S ribosomal subunit DNA to other host cells within the clade as compared to host cells outside the clade. Therefore, host cells that are members of a clade (e.g., the Pichia/Issatchenkia/Saturnospora/Dekkera or Saccharomyces clades) can be identified using the methods of Kurtzman and Robnett.

In certain embodiments of the present disclosure, the recombinant host cells are engineered by introduction of one or more genetic modifications into a Crabtree-positive yeast cell. In certain of these embodiments, the host cell belongs to the Saccharomyces clade. In certain of these embodiments, the host cell belongs to a genus selected from the group comprising Saccharomyces, Schizosaccharomyces, Brettanomyces, Torulopsis, Nematospora and Nadsonia. In certain embodiments, the host cell belongs to the genus Saccharomyces, and in one of these embodiments the host cell is S. cerevisiae.

### 2.1.2 Eukaryotic Cells

In addition to yeast cells, other eukaryotic cells are also suitable for use in accordance with methods of the present disclosure, so long as the engineered host cell is capable of growth and/or product formation, illustrative examples of eukaryotic host cells provided by the present disclosure include, but are not limited to cells belonging to the genera Aspergillus, Crypthecodinium, Cunninghamella, Entomophthora, Mortierella, Mucor, Neurospora, Pythium, Schizochytrium, Thraustochytrium, Trichoderma, and Xanthophyllomyces. Examples of eukaryotic strains include, but are not limited to: Aspergillus niger, Aspergillus oryzae, Crypthecodinium cohnii, Cunninghamella japonica, Entomophthora coronata, Mortierella alpina, Mucor circinelloides, Neurospora crassa, Pythium ultimum, Schizochytrium limacinum, Thraustochytrium aureum, Trichoderma reesei and Xanthophyllomyces dendrorhous.

### 2.1.3 Archaeal Cells

Archaeal cells are also suitable for use in accordance with methods of the present disclosure, and in an embodiment of the present disclosure, the recombinant host cell is an archaeal cell. Illustrative examples of recombinant archaea host cells provided by the present disclosure include, but are not limited to, cells belonging to the genera: Aeropyrum, Archaeoglobus, Halobacterium, Methanococcus, Methanobacterium, Pyrococcus, Sulfolobus, and Thermoplasma. Examples of archaea strains include, but are not limited to Archaeoglobus fulgidus, Halobacterium sp., Methanococcus jannaschii, Methanobacterium thermamtotrophicum, Thermoplasma acidophilum, Thermoplasma valcanium, Pyrococcus horikoshii, Pyrococcus abyssi, and Aeropyrum pernix.

### 2.1.4 Prokaryote Cells

In an embodiment of the present disclosure, the recombinant host cell is a prokaryotic cell. Prokaryotic cells are suitable host cells for construction of recombinant metabolic pathways comprising heterologous enzymes catalyzing production of small-molecule products. Illustrative examples of recombinant prokaryotic host cells include, but are not limited to, cells belonging to the genera Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Pantoea, Phormidium, Pseudomonas, Rhodobacter, Rhodopseudomonas, Rhodospirillum, Rhodococcus, Salmonella, Scenedesmus, Serratia, Shigella, Staphylococcus, Streptomyces, Synechococcus, and Zymomonas. Examples of prokaryotic strains include, but are not limited to, Bacillus subtillis (B. subtilis), Brevibacterium ammoniagenes, Bacillus amyloliquefaciens, Brevibacterium ammoniagenes, Brevibacterium immariophilum, Clostridium acetobutylicum, Clostridium beigerinckii, Corynebacterium glutamicum (C. glutamicum). Enterobacter sakazakii, Escherichia coli (E. coli), Lactobacillus acidophilus, Lactococcus lactis, Mesorhizobium lati, Pantoea ananatis (P. ananatis), Pseudomonas aeruginosa, Pseudomonas mevalonii, Pseudomonas pudita, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus.

### 2.2 Glycolic Acid Pathway

Provided herein in certain embodiments ae recombinant host cells having at least one active glycolic acid pathway from a glycolytic intermediate to glycolate. Recombinant host cells having an active glycolic acid pathway as used herein produce one or more active enzymes necessary to catalyze each metabolic reaction in a glycolic acid pathway, and therefore are capable of producing glycolic acid in measurable yields and/or titers when cultured under suitable conditions. Recombinant host cells having a glycolic acid pathway comprise one or more heterologous nucleic acids encoding glycolic acid pathway enzyme(s) and are capable of producing glycolate.

Recombinant host cells may employ combinations of metabolic reactions for biosynthetically producing the compounds of the present disclosure. The biosynthesized compounds produced by the recombinant host cells include glycolate, glycolic acid, and the intermediates, products and/or derivatives of the glycolic acid pathway. The biosynthesized compounds are produced intracellularly and, in many embodiments, are secreted into the fermentation medium.

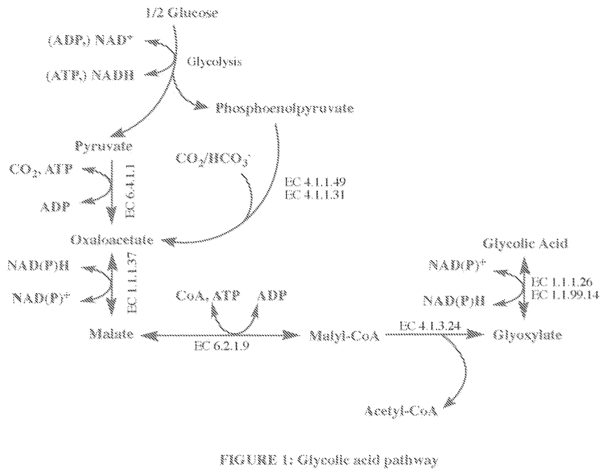

In various embodiments, recombinant host cells of the present disclosure comprise a glycolic acid pathway (FIG. 1) that proceeds via a glycolytic intermediate, such as pyruvate or phosphoenolpyruvate, in various embodiments, these recombinant host cells further comprise a glycolic acid pathway that proceeds via oxaloacetate. The glycolic acid pathway described herein comprises 5 enzymes: (1) an oxaloacetate-forming enzyme, i.e., pyruvate carboxylase (EC #6.4.1.1), phosphoenolpyruvate carboxykinase (EC #4.1.1.49), or phosphoenolpyruvate carboxylase (EC #4.1.1.31), (2) malate dehydrogenase (EC #1.1.1.37); (3) malate-CoA ligase (EC #6.2.1.9); (4) malyl-CoA lyase (EC #4.1.3.24); and (5) glyoxylate reductase. The glycolic acid pathway described herein produces glycolate from glucose with the following balanced, stoichiometric equation:

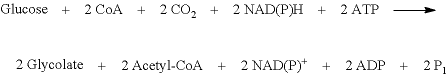

TABLE 1
Enzymes that may function in a glycolic acid pathway

| EC # | Enzyme name | Reaction catalyzed |
|---|---|---|
| 6.4.1.1 | Pyruvate carboxylase | Pyruvate + HCO₃_{^{−}}+ ATP → |
| | | Oxaloacetate + ADP |
| 4.1.1.49 | Phosphoenolpyruvate | Phosphoenolpyruvate + CO₂ → |
| | carboxykinase | Oxaloacetate |
| 4.1.1.31 | Phosphoenolpyruvate | Phosphoenolpyruvate + HCO₃_{^{−}} → |
| | carboxylase | Oxaloacetate |
| 1.1.1.37 | Malate dehydrogenase | Oxaloacetate + Reduced cofactor → |
| | | Malate + Oxidized cofactor |
| 6.2.1.9 | Malate-CoA ligase | Malate + CoA + ATP → Malyl-CoA + |
| | | ADP |
| 4.1.3.24 | Malyl-CoA lyase | Malyl-CoA → Glyoxylate + Acetyl-CoA |
| 1.1.1.26 or | Glyoxylate reductase | Glyoxylate + Reduced cofactor → |
| 1.1.99.14 | | Glycolate + Oxidized cofactor |

The stoichiometric yield for the reaction (i.e., not accounting for biomass formation and aerobic respiration for generation of ATP for cellular housekeeping) is 0.85 g of glycolic acid for every g of glucose consumed. Importantly, the reaction also generates acetyl-CoA (I mole acetyl-CoA per mole glycolic acid), which is useful for a number of reasons. The acetyl-CoA generated can be used to grow and/or maintain the biomass, generate ATP, or it can be converted into additional glycolic acid. Converting acetyl-CoA to glycolic acid is a particularly attractive option in that it increases the stoichiometric yield to 1.69 g-glycolic acid per g-glucose consumed (eq. to 4 moles-glycolic acid per mole-glucose).

The aforementioned glycolic acid pathway is calculated to thermodynamically favor the conversion of pyruvate to glycolate. The advantaged thermodynamics of the pathway will help to achieve high glycolic acid yields, titers and/or productivities, and/or high downstream product yields, titers and/or productivities. The conversion of glucose to glycolate using the glycolic acid pathway described herein has a calculated change in Gibbs free energy of −166.3+/−13.8 kJ/mol (i.e., Δ_{f}G^{m} calculated at 1 mM metabolite concentrations, 25° C., pH 7.0, and 0.1 M ionic strength), a negative value indicative of a strong driving force that pushes the reaction to completion.

The glycolic acid pathway (FIG. 1) converts a glycolytic intermediate (i.e., pyruvate or phosphoenolpyruvate) to glycolate via oxaloacetate. In various embodiments, recombinant host cells comprise enzymes of the glycolic acid pathway and are capable of producing glycolate. In some embodiments, the recombinant host cells further comprise a biosynthetic pathway converting the acetyl-CoA (resulting from malyl-CoA lyase step) to additional glycolic acid.

The glycolic acid pathway of the present disclosure comprises 5 steps that take place in the cytosol (FIG. 1) and converts 1 molecule of a glycolytic intermediate (i.e., pyruvate or phosphoenolpyruvate) to 1 molecule of glycolate. In the first step, a glycolytic intermediate is converted to oxaloacetate. In embodiments where the glycolytic intermediate is pyruvate, pyruvate carboxylase (EC #6.4.1.1) converts 1 molecule of pyruvate, 1 molecule of bicarbonate and 1 molecule of ATP to 1 molecule of oxaloacetate and 1 molecule of ADP. In many embodiments where the glycolytic intermediate is phosphoenolpyruvate, phosphoenolpyruvate carboxykinase (EC #4.1.1.49) converts 1 molecule of phosphoenolpyruvate and 1 molecule of carbon dioxide to 1 molecule of oxaloacetate. In other embodiments where the glycolytic intermediate is phosphoenolpyruvate, phosphoenolpyruvate carboxylase (EC #4.1.1.31) converts 1 molecule of phosphoenolpyruvate and 1 molecule of bicarbonate to 1 molecule of oxaloacetate. In the second step, malate dehydrogenase (EC #1.1.137) converts 1 molecule of oxaloacetate and 1 molecule or NAD(P)H to 1 molecule of malate and 1 molecule of NA(P)⁺. In the third step, malate-CoA ligase (EC #6.2.1.9) converts 1 molecule of malate, 1 molecule of CoA and 1 molecule of ATP to 1 molecule of malyl-CoA and 1 molecule of ADP. In some embodiments, the malate-CoA ligase in this third step produces AMP instead of ADP. In the fourth step, malyl-CoA lyase (EC #4.1.3.24) converts 1 molecule of malyl-CoA to 1 molecule of glyoxylate and 1 molecule of acetyl-CoA. In the fifth step, glyoxylate reductase (EC #1.1.1.26) converts 1 molecule of glyoxylate and 1 molecule of NAD(P)H to 1 molecule of glycolate and 1 molecule of NAD(P)⁺.

In certain embodiments, recombinant host cells comprise one or more heterologous nucleic acids encoding 1, 2, 3, 4, 5, 6, or all 7, of the aforementioned glycolic acid pathway enzymes (Table 1) or any combination thereof, wherein the heterologous nucleic acids are expressed in sufficient amounts to produce glycolate. In various embodiments, recombinant host cells may comprise multiple copies of a single heterologous nucleic acid and/or multiple copies of 2 or more heterologous nucleic acids. Recombinant host cells comprising multiple heterologous nucleic acids may comprise any number of heterologous nucleic acids.

The present disclosure also provides consensus sequences (defined above) useful in identifying and/or constructing the glycolic acid pathway suitable for use in accordance with the methods of the present disclosure. In various embodiments, these consensus sequences comprise active site amino acid residues believed to be necessary (although the subject matter provided by the present disclosure is not to be limited by any theory of mechanism of action) for substrate recognition and reaction catalysis, as described below. Thus, an enzyme encompassed by a consensus sequence provided herein has an enzymatic activity that is identical, or essentially identical, or at least substantially similar with respect to ability to catalyze the reaction performed by one of the enzymes exemplified herein. For example, a malate-CoA ligase encompassed by a malate-CoA ligase consensus sequence provided herein has an enzymatic activity that is identical, or essentially identical, or at least substantially similar with respect to ability to convert 1 molecule of malate, 1 molecule of CoA and 1 molecule of ATP to 1 molecule of malyl-CoA and 1 molecule of either ADP or AMP. As noted above, any protein substantially homologous to malate-CoA ligase as described herein can be used in a host cell of the present disclosure. Further, any protein that shares the specific function of malate-CoA ligase as described herein can be used in a host cell of the disclosure despite comprising insufficient sequence homology with the malate-CoA ligase consensus sequence.

### 2.2.1 Oxaloacetate-Forming Enzymes

The first step of the glycolic acid pathway comprises converting a glycolytic intermediate into oxaloacetate. In various embodiments of the present disclosure, recombinant host cells comprise one or more heterologous nucleic acids encoding an oxaloacetate-forming enzyme wherein the oxaloacetate-forming enzyme is selected from the group comprising pyruvate carboxylase (EC #6.4.1.1), phosphoenolpyruvate carboxykinase (EC #4.1.1.49), and phosphoenolpyruvate carboxylase (EC #4.1.1.31), wherein said recombinant host cells are capable of producing glycolic acid. In some embodiments, the recombinant host cells comprise one or more heterologous nucleic acids encoding 1, 2, or all 3 of the aforementioned oxaloacetate-forming enzymes. In many embodiments, the oxaloacetate-forming enzyme is derived from a prokaryotic source. In other embodiments, the oxaloacetate-forming enzyme is derived from a eukaryotic source.

### 2.2.1.1 Pyruvate Carboxylase

The pyruvate carboxylase (EC #6.4.1.1) described herein catalyzes the conversion of 1 molecule of pyruvate, 1 molecule of bicarbonate (HCO₃⁻) and 1 molecule of ATP to 1 molecule of oxaloacetate and 1 molecule of ADP. Any enzyme is suitable for use in accordance with the present disclosure so long as the enzyme is capable of catalyzing said pyruvate carboxylase reaction.

In many embodiments, the pyruvate carboxylase is derived from a bacterial source. In many of these embodiments, the pyruvate carboxylase is derived from a host cell belonging to a genus selected from the group comprising Geobacillus, Rhizobium, Pseudomonas, Mycobacterium, Staphylococcus, Arthrobacter, Sinorhizobium and Methanocaldococcus. Non-limiting examples of bacterial pyruvate carboxylase comprise Geobacillus thermodenitrificans UniProt ID: A4ILW8. Geobacillus thermodenitrificans UniProt ID: Q05FZ3, Geobacillus stearothermophilus UniProt ID: P94448, Geobacillus stearothermophilus UniProt ID: Q8LIN9, Rhizobium eth UniProt ID: Q2K340, Pseudomonas fluorescence UniProt ID: C3KEC5, Pseudomonas florescence UniProt ID: E2XMN3, Pseudomonas fluorescence UniProt ID: V7E6C6, Pseudomonas fluorescence UniProt ID: K0WNR6, Pseudomonas fluorescence UniProt ID: L7HKS9, Pseudomonas fluorescence UniProt ID: J2Y9J8, Pseudomonas fluorescence UniProt ID: U1TDW3, Pseudomonas fluorescence UniProt ID: I4K2J5, Pseudomonas fluorescence UniProt ID: G8QB75, Methanocaldococcus jannaschii UniProt ID: Q58626 and Q58628, Mycobacterium smegmatis UniProt ID: L8FHY2, Mycobacterium smegmatis UniProt ID: 17G857, Mycobacterium smegmatis UniProt ID: 17FNQ9, Mycobacterium smegmatis UniProt ID: A0R6R9, Mycobacterium smegmatis UniProt ID: L8FKA4, Mycobacterium smegmatis UniProt ID: L8FB92, Mycobacterium smegmatis UniProt ID: Q9F843, Mycobacterium smegmatis UniProt ID: A0QV14, and Mycobacterium smegmatis UniProt ID: L8FBY1.

In many embodiments, the pyruvate carboxylase is derived from a eukaryotic source. In many of these embodiments, the pyruvate carboxylase is derived from a host cell belonging to a genus selected from the group comprising Aspergillus, Paecilomyces, Pichia, Saccharomyces, Phycomyces, Emiliania. Non-limiting examples of eukaryotic pyruvate carboxylase comprise Aspergillus niger UniProt ID: Q9HES8, Aspergillus terreus UniProt ID: 093918, Aspergillus oryzae UniProt ID: Q2UGL1, Aspergillus oryzae UniProt ID: A0A099P575; Aspergillus oryzae UniProt ID: A0A1V2LT98, Aspergillus oryzae UniProt ID: 18TVE3, Aspergillus oryzae UniProt ID: A0A1S9DZ43, Paecilomyces variotii UniProt ID: V5FW17, Pichia kudriavzevii UniProt ID: A0A099P757, Pichia kudriavzevii UniProt ID: A0A1V2LT98, Pichia kudriavzevii UniProt ID: A0AIZ8JRB6, Saccharomyces cerevisiae UniProt ID: P11154, Saccharomyces cerevisiae UniProt ID: P32327, Phycomyces blakesleeanus UniProt ID: A0A167KQN5, Phycomyces blakesleeanus UniProt ID: A0A167L0T9, Emiliania huxleyi UniProt ID: B9X0T8.

In some embodiments, the pyruvate carboxylase is the S. cerevisiae pyruvate carboxylase 2 (abbv. ScPYC2; UniProt ID: P32327; SEQ ID NO: 1). In some embodiments, the pyruvate carboxylase is the S. cerevisiae pyruvate carboxylase 1 (abbv. ScPYC1; UniProt ID: P11154; SEQ ID NO: 2). In some embodiments, the pyruvate carboxylase is the P. kudriavzevii pyruvate carboxylase 1 (abbv. PkPYC1; UniProt ID: A0A099P575; SEQ ID NO: 8). In some embodiments, the pyruvate carboxylase is the P. kudriavzevii pyruvate carboxylase 2 (abbv. PkPYC2; UniProt ID: A0A1V2LT99; SEQ ID NO: 9). In some embodiments, the pyruvate carboxylase is the Aspergillus oryzae pyruvate carboxylase (abbv. AoPYC; UniProt ID: Q2UGL1; SEQ ID NO: 53). In some embodiments, the pyruvate carboxylase is the P. kudriavzevii pyruvate carboxylase 3 (abbv. PkA0A1Z8JRB6; UniProt ID: A0A1Z8JRB6; SEQ ID NO: 10). In some embodiments, the pyruvate carboxylase is the Aspergillus oryzae pyruvate carboxylase 2 (abbv. AoPYC2; UniProt ID: 18TVE3; SEQ ID NO: 68). In some embodiments, the pyruvate carboxylase is the Aspergillus oryzae pyruvate carboxylase 3 (abbv. AoPYC3; UniProt ID: A0A1S9DZ43; SEQ ID NO: 69).

In many embodiments, recombinant host cells comprise one or more heterologous nucleic acids encoding a pyruvate carboxylase wherein said recombinant host cells are capable of producing glycolic acid. In various embodiments, proteins suitable for use in accordance with methods of the present disclosure have pyruvate carboxylase activity and comprise an amino acid sequence with at least 6%, at least 70%, at least 80%, at least 90%, or at least 95% sequence homology with SEQ ID NO: 1, SEQ ID NO: 2. SEQ ID NO: 8. SEQ ID NO: 9. SEQ ID NO: 10. SEQ ID NO: 53, SEQ ID NO: 68, or SEQ ID NO: 69. In many embodiments, the recombinant host cell is a P. kudriavzevii strain.

### 2.2.1.2 Phosphoenolpyruvate Carboxykinase

The phosphoenolpyruvate carboxykinase (PCK)(EC #4.1.1.49) described herein catalyzes the conversion of 1 molecule of phosphoenolpyruvate and 1 molecule of CO₂ to 1 molecule of oxaloacetate. Any enzyme is suitable for use in accordance with the present disclosure so long as the enzyme is capable of catalyzing said PCK reaction.

Recombinant host cells comprising one or more heterologous nucleic acids encoding a PCK of the present disclosure have an increase in glycolic acid titer and/or yield as compared to parental or control cells that do not comprise said heterologous nucleic acid(s). In some embodiments, recombinant host cells comprising one or more heterologous nucleic acids encoding a PCK produce an increased glycolic acid titer in fermentations as compared to parental or control cells that do not comprise said heterologous nucleic acid(s). In some embodiments, the glycolic acid titer is increased by 0.5 g/l, 1 g/l, 2.5 g/l, 5 g/l, 7.5 g/l, 10 g/l, or more than 10 g/l. In some embodiments, recombinant host cells comprising one or more heterologous nucleic acids encoding a PCK have increased glycolic acid yield as compared to parental or control cells that do not comprise said heterologous nucleic acid(s). In some embodiments, the glycolic acid yield is increased by 0.5%, 1%, 2.5%, 5%, 7.5%, 10%, or more than 10% (g-glycolic acid/g-substrate, or g-downstream product/g-substrate).

In many embodiments, the PCK is derived from a bacterial source. In many of these embodiments, the PCK is derived from a host cell belonging to a genus selected from the group comprising Actinobacillus, Escherichia, Anaerobiaspirillum, Bacillus, Corynebacterium, Cupriavidus, Leishmania, Rhodopseudomonas, Ruminiclostridium, Ruminococcus, Salinivibrio, Selenomonas, Sinorhizobium, Staphylococcus, Mannheimia, Haemophilus, and Thermus. Non-limiting examples of bacterial PCK comprise Actinobacillus ficoidea UniProt ID: Q6W6X5, Anaerobiospirillum succiniciproducens UniProt ID: O09460, E. coli UniProt ID: P22259, Anaerobiospirillum succiniciproducens UniProt ID: O09460, Actinobacillus succinogenes UniProt ID: A6VKV4, Mannheimia succiniciproducens UniProt ID: Q65Q60, Ruminococcus albus UniProt ID: B3Y4D3, Selenomonas ruminantium UniProt ID: 083023, Thermus thermophiles UniProt ID: Q5SLL5, and Haemophilus influenzae UniProt ID: A5UDR5.

In many embodiments, the PCK is derived from a eukaryotic source. In many of these embodiments, the pyruvate carboxylase is derived from a host cell belonging to a genus selected from the group comprising Alternanthera, Ananas, Arabidopsis, Clusia, Cucumis, Digitaria, Embryophyta, Hordeum, Iris, Laminaria, Megathyrus, Mus, Nicotiana, Oryza, Pisum, Plasmodium, Prunus, Saccharomyces, Skeletonema, Solanum, Solenostemon, Sorghum, Tillandsia, Trypanosoma, Udotea, Urochloa, Vitis, Pichia, Aspergillus, Zoysia and Zea. In some embodiments, the PCK is derived from a fungal source. Non-limiting examples of eukaryotic PCK comprise Arabidopsis thaliana UniProt ID: Q93VK0, Plasmodium falciparum UniProt ID: Q9U750, Saccharomyces cerevisiae UniProt ID: PI0963, Pichia kudriavzevii UniProt ID: A0A099NX43, and Zoysia japonica UniProt ID: Q5KQS7.

In some embodiments, the PCK is the Saccharomyces cerevisiae phosphoenolpyruvate carboxykinase (abbv. ScPCK1; UniProt ID: P10963; SEQ ID NO: 54). In some embodiments, the PCK is the Pichia kudriavzevii phosphoenolpyruvate carboxykinase (abbv. PkPCK; UniProt ID: A0A099NX43; SEQ ID NO: 55).

In many embodiments, recombinant host cells comprise one or more heterologous nucleic acids encoding a PCK wherein said recombinant host cells are capable of producing glycolic acid. In various embodiments, proteins suitable for use in accordance with methods of the present disclosure have pyruvate carboxylase activity and comprise an amino acid sequence with at least 60%, at least 70%, at least 80%, at least 90%, or at least 95% sequence homology with SEQ ID NO: 54, or SEQ ID NO: 55. In many embodiments, the recombinant host cell is a P. kudriavzevii strain.

### 2.2.1.3 Phosphoenolpyruvate Carboxylase

The phosphoenolpyruvate carboxylase (PPC)(EC #4.1.1.31) described herein catalyzes the conversion of 1 molecule of phosphoenolpyruvate and 1 molecule of HCO; to 1 molecule of oxaloacetate. Any enzyme is suitable for use in accordance with the present disclosure so long as the enzyme is capable of catalyzing said PPC reaction.

Recombinant host cells comprising one or more heterologous nucleic acids encoding a PPC of the present disclosure have an increase in glycolic acid titer and/or yield as compared to parental or control cells that do not comprise said heterologous nucleic acid(s). In some embodiments, recombinant host cells comprising one or more heterologous nucleic acids encoding a PPC produce an increased glycolic acid titer in fermentations as compared to parental or control cells that do not comprise said heterologous nucleic acid(s). In some embodiments, the glycolic acid titer is increased by 0.5 g/l, 1 g/l, 2.5 g/l, 5 g/l, 7.5 g/l, 10 g/l, or more than 10 g/l. In some embodiments, recombinant host cells comprising one or more heterologous nucleic acids encoding a PPC have increased glycolic acid yield as compared to parental or control cells that do not comprise said heterologous nucleic acid(s). In some embodiments, the glycolic acid yield is increased by 0.5%, 1%, 2.5%, 5%, 7.5%, 10%, or more than 10% (g-glycolic acid/g-substrate, or g-downstream product/g-substrate).

In many embodiments, the PPC is derived from a prokaryotic source. In many of these embodiments, the PPC is derived from a host cell belonging to a genus selected from the group comprising Acetobacter, Bradyrhizoibum, Brevibacterium, Chlamydomonas, Clostridium, Escherichia, Mycobacterium, Hyphomicrobiium, Methanothermobacter, Methanothermus, Photobacterium, Pseudomonas, Rhodospeudomonas, Roseobacter, Starkeya, Streptomyces, Thermosynechococcus, Thiobacillus, Halothiobacillus, Thermus, and Corynebacterium. Non-limiting examples of bacterial PPC comprise Clostridium perfingens UniProt ID: Q8XLE8, Escherichia coli UniProt ID: P00864, Mycobacterium tuberculosis UniProt ID: P9WIH3, Corynebacterium glutamicum UniProt ID: P12880, and Thermosynechococcus vulcanus UniProt ID: P0A3X6.

In many embodiments, the PPC is derived from a eukaryotic source. In many of these embodiments, the pyruvate carboxylase is derived from a host cell belonging to a genus selected from the group comprising Alternanthera, Amaranthus, Ananas, Annona, Arabidopsis, Atriplex, Beta, Brachiaria, Bracssica, Bryophyllum, Cicer, Citrus, Coccochloris, Colealaenia, Commelina, Crassula, Cucumis, Digitaria, Echinochloa, Embryophyta, Euglena, Flaveria, Gallus, Glycine, Hakea, Haloxylon, Helianthus, Hordeum, Hydrilla, Iris, Kalanchoe, Lilium, Lotus, Lupinus, Malus, Medicago, Megathyrus, Mesembryanthemum, Molinema, Monoraphidium, Musa, Nicotiana, Oryza, Panicum, Persea, Phaeodactylum, Pinus, Pisum, Plasmodium, Portulaca, Ricinus, Saccharomyces, Solanum, Sorghum, Spinacia, Steinchisma, Starkeya, Umbilicus, Vicia, Xylosalsola, and Zea. In some embodiments, the PPC is derived from a fungal source, Non-limiting examples of eukaryotic PPC comprise Alternanthera ficoidea UniProt ID: Q1XAT8, Arabidopsis thaliana UniProt ID: Q5GM68, Arabidopsis thaliana UniProt ID: Q84VW9, Arabidopsis thaliana UniProt ID: Q8GVE8, Arabidopsis thaliana UniProt ID: Q9MAHO, Gossypium hirsutum UniProt ID: O23946, and Pinus halepensis UniProt ID: Q9M3Y3.

In some embodiments, the PPC is the Escherichia coli phosphoenolpyruvate carboxylase (abbv. EcPPC; UniProt ID: P00864; SEQ ID NO: 58). In some embodiments, the PPC is the Mycobacterium tuberculosis phosphoenolpyruvate carboxylase (abbv. MtPCKG: UniProt ID: P9WIH3; SEQ ID NO: 59). In some embodiments, the PPC is the Corynebacterium glutamicum phosphoenolpyruvate carboxylase (abbv. CgPPC; UniProt ID: P12880; SEQ ID NO 60).

In many embodiments, recombinant host cells comprise one or more heterologous nucleic acids encoding a PPC wherein said recombinant host cells are capable of producing glycolic acid, in various embodiments, proteins suitable for use in accordance with methods of the present disclosure have PPC activity and comprise an amino acid sequence with at least 60%, at least 70%, at least 80%, at least 90%, or at least 95% sequence homology with SEQ ID NO: 58, SEQ ID NO: 59, or SEQ ID NO: 60. In many embodiments, the recombinant host cell is a P. kudriavzevii strain.

In some embodiments, recombinant host cells comprise one or more heterologous nucleic acids encoding a PPC wherein the PPC was mutagenized towards an altered enzyme characteristic such as altered substrate affinity, cofactor affinity, altered reaction rate, and/or altered inhibitor affinity. In these embodiments, the PPC variant is a product of one or more protein engineering cycles. In these embodiments, the PPC variant comprises one or more point mutations. In these embodiments, proteins suitable for use in accordance with methods of the present disclosure have PPC activity and comprise an amino acid sequence with at least 60%, at least 70%, at least 80%, at least 90%, or at least 95% sequence homology with SEQ ID NO: 58, SEQ ID NO: 59, or SEQ ID NO: 60 In some of these embodiments, the PPC variant has decreased affinity for allosteric inhibitors, Non-limiting examples of allosteric inhibitors of PPC include aspartate, acetyl-CoA, and malate. For example, in EcPPC (SEQ ID NO: 58), the allosteric binding site for aspartate is located 20 angstroms away from the catalytic site and the 4 residues involved in binding aspartate are Lys773, Arg832, Arg587, and Asn881. In some embodiments, proteins with at least 60% sequence homology with SEQ ID NO: 58 comprise a mutation at one, some, or all of these amino acids (Lys773, Arg832, Arg587, and Asn8l) to decrease binding of aspartate. In embodiments wherein the recombinant host cells comprise one or more heterologous nucleic acids encoding such a mutagenized PPC, the recombinant host cells produce glycolic acid at a titer and/or yield that is higher than recombinant host cells lacking said mutagenized PPC.

The PPC consensus sequence #3 (SEQ ID NO: 57) provides the sequence of amino acids in which each position identifies the amino acid (if a specific amino acid is identified) or a subset of amino acids (if a position is identified as variable) most likely to be found at a specific position in a PPC. Many amino acids in consensus sequence #3 (SEQ ID NO: 57) are highly conserved and PPCs suitable for use in accordance with the methods of the present disclosure will comprise a substantial number, and sometimes all, of these highly conserved amino acids at positions aligning with the location of the indicated amino acids in consensus sequence #3 (SEQ ID NO: 57). In various embodiments, proteins suitable for use in accordance with the methods of the present disclosure have phosphoenolpyruvate carboxylase activity and comprise an amino acid sequence with at least at least 60%, at least 65%, or at least 70 sequence homology with consensus sequence #3 (SEQ ID NO: 57). For example, the EcPPC sequence (SEQ ID NO: 58) is 71% homologous to consensus sequence #3 (SEQ ID NO: 37), and is therefore encompassed by consensus sequence #3 (SEQ ID NO: 57).

Highly conserved amino acids in consensus sequence #3 SEQ ID NO: 57 are M1, Y5, N11, S13, M14, L15, G16, L19, G20, T22, I23, A26, G28, E36, I38, R39, L41, S42, R46, G48, R53, L56, P70, V71, A72, R73, A74, F75, Q77, F78, 179, N80, L81, N83, A85, E86, Q87, Y88,191, S92, L111, V125, E131, L132, V133, L134, T135, A136, H137, P138, T139, E140, R143, R144, K149, N154, C156, L157, L160, E169, L177, L180, A182, W185, H186, I190, R191, R194, P195, P197, E200, A201, K202, W203, G204, A206, E209, N210, S211, L212, W213, P217, L220, R221, L235, P241, W247, M248, G249, G250, D251, R252, D253, G254, N255, P256, V258, T259, T263, R271, W272, K273, A274, L277, L279, D281, L285, E288, L289, S290, G303, E309, P310, Y311, R312, K316, R319, L322, T325, L351, W352, P354, L355, C358, Y359, S361, L362, C365, G366, M367, I369, I370, A371, G373, L375, L376, D377, L379, R381, F385, G386, L389, D393, R395, Q396, E397, S398, T399, H401, E407, Y411, G415, D416, Y417, W420, E422, K425, F428, L429, E432, L433, S435, R437, P438, L439, P441, W444, P446, S447, E452, T456, C457, Y471, 1473, S474, M475, A476, S480, D481, V482, L483, A484, V485, L487, L488, L489, E491, G493, V500, P502, L503, P504, E505, T506, L507, D509, L510, L520, W525, Y526, R527, I530, Q534, M535, V536, M537, I538, G539, Y540, S541, D542, S543, A544, K545, D546, A547, G548, M550, A552, W554, A555, Q556, Y557, A559, L563, L574, T575, L576, F577, H578, G579, R580, G581, G582, I584, G585, R586, G587, G588, A389, P590, A591, H592, A594, L595, L596, S597, Q598, P599, P600, S602, L603, K604, G606, L607, R608, V609, T610, E611, Q612, G613, E614, M615, I616, R617, F618, K619, G621, L622, P623, Y633, A636, L639, E639, A640, N641, L642, L643, P644, P645, P646, P648, K649, W652, M656, L659, S660, S663, C664, Y667, R668, R672, F677, V678, Y680, F681, R682, A684, T685, P686, E687, E689, L690, K692, L693, P694, L695, 0696, S697, R698, P699, A700, K701, R702, P704, G706, G707, V708, E709, L711, R712, A713, I714, P715, W716, I717, F718, W720, Q722, N723, R724, L725, L727, P728, A729, W730, L731, G732, A733, G734, G744, M752, W756, P757, F758, F759, T761, R762, M765, L766, E767, M768, V769, K772, Y781, D782, L785, L790, W791, L793, G794, L797, R798, D804, I805, V808, L809, L817, M818, P822, W823, I828, L830, R831, N832, Y834, P837, L838, N839, L841, Q842, E844, L845, L846, R848, R850, E960, A862, L863, M864, I867, G869, A871, G873, M874, R875, N876, T877, and G878. Enzymes homologous to SEQ ID NO: 57 will contain a majority of these conserved amino acids at positions aligning with (i.e., corresponding to) the highly conserved amino acids in SEQ ID NO: 57. In various embodiments, PPC enzymes homologous to SEQ ID NO: 57 comprise at least 60%, at least 70%, at least 80%, at least 85%, at least 90%, at least 95%, or sometimes all of these highly conserved amino acids at positions corresponding to the highly conserved amino acids identified in SEQ ID NO: 57. In some embodiments, each of these highly conserved amino acids are found in a desired PPCs as provided, for example, in SEQ ID NO: 58.

### 2.2.2 Malate Dehydrogenase

The malate dehydrogenase (EC #1.1.1.37) described herein catalyzes the conversion of 1 molecule of pyruvate, 1 molecule of oxaloacetate and 1 molecule of NAD(P)H to 1 molecule of malate and 1 molecule of NAD(P)⁺. Any enzyme is suitable for use in accordance with the present disclosure so long as the enzyme is capable of catalyzing said malate dehydrogenase reaction. In some embodiments, the malate dehydrogenase preferentially utilizes NADH instead of NADPH.

Recombinant host cells comprising one or more heterologous nucleic acids encoding a malate dehydrogenase of the present disclosure have an increase in glycolic acid titer and/or yield as compared to parental or control cells that do not comprise said heterologous nucleic acid(s). In some embodiments, recombinant host cells comprising one or more heterologous nucleic acids encoding a malate dehydrogenase produce an increased glycolic acid titer in fermentations as compared to parental or control cells that do not comprise said heterologous nucleic acid(s). In some embodiments, the glycolic acid titer is increased by 0.5 g/l, 1 g/l, 2.5 g/l, 5 g/l, 7.5 g/l, 10 g/l, or more than 10 g/l. In some embodiments, recombinant host cells comprising one or more heterologous nucleic acids encoding a malate dehydrogenase have increased glycolic acid yield as compared to parental or control cells that do not comprise said heterologous nucleic acid(s). In some embodiments, the glycolic acid yield is increased by 0.5%, 1%, 2.5%, 5%, 7.5%, 10%, or more than 10% (g-glycolic acid/g-substrate, or g-downstream product/g-substrate).

In many embodiments, the malate dehydrogenase is derived from a bacterial source. In many of these embodiments, the malate dehydrogenase is derived from a host cell belonging to a genus selected from the group comprising Thermus, Beggiatoa, Rhodopseudomonas, Mycobacterium, Leishmania, Acinetobacter, Corynebacterium, Methanospirillum, Vulcanithermus, Syntrophobacter, Macromonas, Sulfolobus, Streptomyces, Comomonas, Bacillus, Moritella, Haemophilus, and Escherichia. Non-limiting examples of bacterial enzymes comprise Thermus themophilus UniProt ID: P10584, Corynebacterium glutamicum UniProt ID: Q8NN33, Leishmania mexicana UniProt ID: Q0QW09, Escherichia coli UniProt ID: P61889, Moritella sp. UniProt ID: Q7X3X5, and Streptomyces coelicolor UniProt ID: Q9K3J3.

In many embodiments, the malate dehydrogenase is derived from an archaeal source. In many of these embodiments, the malate dehydrogenase is derived from a host cell belonging to a genus selected from the group comprising Pyrobaculum, Thermoplasma, and Archaeoglobus. Non-limiting examples of archaeal enzymes comprise Archaeoglobus fulgidus UniProt ID; O08349, and Pyrobaculum calidifontis UniProt ID: A3MWU9.

In many embodiments, the malate dehydrogenase is derived from a eukaryotic source. In many of these embodiments, the malate dehydrogenase is derived from a host cell belonging to a genus selected from the group comprising Pichia, Saccharomyces, Mesenbryanthemum, Sphyraena, Echinococcus, Trypanosoma, Trichomonas, Geophagus, Tritrichomonas Spinacia, Sus, Chlonorchis, Rasamsonia, Phytophthora, Eugelena, Ratus. Drosophila, Candida, Hoplias, Dictyostelium, Phycomyces, Plasmodium, Malassezia, Gallus, Drosophila, Taenia, Triticum, Physarum, Ananas, Aspergillus, Vigna, Zea, and Malus. Non-limiting examples of eukaryotic enzymes comprise Echinococcus granulosus UniProt ID: Q704F5, Mesembryanthemum crystallimum UniProt ID: Q24047, Sphyraena ideates UniProt ID: Q90YZ7, Sphyraena idiastes UniProt ID: Q90YZ8, Sphyraena idiastes UniProt ID: Q90YZ9, Trypanosoma brucei UniProt ID: O15769, Trichomonas gallinae UniProt ID: Q9ZF99, Rasamsonia emersonii UniProt ID: Q8TG27, Rasamsonia emersonii UniProt ID: Q8X1C8, Plasmodium falciparum UniProt ID: Q6VVP7, Taenia solium UniProt ID: F1C714, Mesenbryanthemum crystallimum Uniprot ID: O24047. Mesenbryanthemum crystallimum UniProt ID: Q9ZF99, Triticum aestivum UniProt ID: A3KLL4. S. cerevisae UniProt ID: P32419, Aspergillus oryzae UniProt ID: Q2U9I9. Aspergillus oryzae UniProt ID: A0A1S9D9V2, Aspergillus oryzae UniProt ID: I8U0T6, and Malus domestica UniProt ID: A3DSX0.

In some embodiments, the malate dehydrogenase is derived from the S. cerevisae peroxisomal malate dehydrogenase (abbv. ScMDH3; UniProt ID: P32419; SEQ ID NO: 4). In some embodiments, the malate dehydrogenase is derived from the Aspergillus oryzae malate dehydrogenase (abbv, AoMDH; UniProt ID: Q2U919 SEQ ID NO: 70). In some embodiments, the malate dehydrogenase is derived from the Aspergillus oryzae malate dehydrogenase 2 (abbv. AoMDH2; UniProt ID: A0A1S9D9V2, SEQ ID NO: 71). In some embodiments, the malate dehydrogenase is derived from the Aspergillus oryzae malate dehydrogenase 3 (abbv, AoMDH3; UniProt ID: I8U0T6; SEQ ID NO: 64). In some of these embodiments, the malate dehydrogenase further comprises a targeting signal deletion. In some of these embodiments, the targeting signal is a peroxisomal targeting signal.

In many embodiments, recombinant host cells comprise one or more heterologous nucleic acids encoding a malate dehydrogenase wherein said recombinant host cells are capable of producing glycolic acid, in various embodiments, proteins suitable for use in accordance with methods of the present disclosure have malate dehydrogenase activity and comprise an amino acid sequence with at least 60%, at least 70%, at least 80%, at least 90%, or at least 95% sequence homology with SEQ ID NO: 4, SEQ ID NO: 64, SEQ ID NO: 70, or SEQ ID NO: 71. In many embodiments, the recombinant host cell is a P. kudriavzevii strain.

### 2.2.3 Malate-CoA Ligase

The malate-CoA ligase (EC #6.2.1.9) described herein catalyzes the conversion of 1 molecule of malate, 1 molecule of CoA and 1 molecule of ATP to 1 molecule of malyl-CoA and 1 molecule of ADP. In some embodiments, the malate-CoA ligase generates ADP. In other embodiments, the malate-CoA ligase generates AMP instead of ADP. Any enzyme is suitable for use in accordance with the present disclosure so long as the enzyme is capable of catalyzing said malate-CoA ligase reaction. In some embodiments, the malate-CoA ligase is a dimer. In some of these embodiments, the malate-CoA ligase is a heterodimer. Practitioners skilled in the art will appreciate that malate-CoA ligase is synonymous with malyl-CoA synthase and these terms are used interchangeably in this disclosure.

A malate-CoA ligase is necessary in the 3-hydroxypropionate cycle required for acetyl-CoA production and serine/ethylmalonyl-CoA pathways in C₁ carbon assimilation in methylotrophic bacteria; thus, the malate-CoA ligases is believed to have evolved properties such as reasonable reaction rates and substrate specificity that are suitable for use in the glycolic acid pathway described herein. The malate-CoA ligase reaction in step 3 of said glycolic acid pathway has a calculated ΔG^{m} of −6.6 kJ/mol, indicative of enzyme flux in the forward direction under equilibrium conditions. In some embodiments, recombinant host cells comprise one or more heterologous nucleic acids encoding a malate-CoA ligase wherein said recombinant host cells are capable of producing glycolate.

In some embodiments, the heterodimeric malate-CoA ligase is derived from a heterodimeric succinate-CoA ligase (EC #6.2.1.4 or EC #6.2.1.5), Malate-CoA ligase and succinate-CoA ligase are thought to share an evolutionary ancestor, and previous studies have reported succinate-CoA ligase activity toward a broad range of substrates including malate (see, for example, Nolte et al, Appl. Environ. Microbiol. 2017). Thus, said succinate-CoA ligases capable of accepting malate as a substrate are also malate-CoA ligases, and as such said malate-CoA ligases are said to be derived from a succinate-CoA ligase. In some embodiments, recombinant host cells of the present disclosure comprise one or more heterologous nucleic acids encoding a malate-CoA ligase derived from a succinate-CoA ligase. Likewise, other classes of acid thiol ligases (EC #s 6.2.1.X, where X is any number) may also have malate-CoA ligase activity, and as such are suitable for use in accordance with the methods of the present disclosure. In many embodiments, the malate-CoA ligase is derived from an acid thiol ligase, which comprises malate-CoA ligase (EC #6.2.1.9), succinate-CoA ligase (EC #6.2.1.4 or EC #6.2.1.5), acetate-CoA ligase (EC #6.2.1.1), butyrate-CoA ligase (EC #6.2.1.2), and acetoacetate-CoA ligase (EC #6.2.1.16) wherein said recombinant host cells are capable of producing glycolate and the malate-CoA ligase is able to catalyze said malate-CoA ligase reaction. In many embodiments, the malate-CoA ligase has one or more mutations as compared to the parental acid thiol ligase protein sequence from which the malate-CoA ligase was derived. In some embodiments, the one or more mutations lies in the active site, catalytic site, or substrate binding site.

Methods for identifying and improving enzymes towards desired malate-CoA ligase activity are disclosed below in section 2.3. The methods described below for protein mutagenesis, identification, expression, purification, and characterization am methods widely-practiced by practitioners skilled in the art, who will appreciate that a wide variety of commercial solutions are available for such endeavors. Briefly, exemplary methods that may be used (as described in section 2.3) include random and rational mutagenesis methods such as site-specific mutagenesis, error-prone PCR, and directed evolution. Libraries of mutated genes or strains are generated, and mutagenized proteins are characterized, and integrated into the genome of recombinant host cells for further strain characterization. Practitioners in the art will appreciate that the methods disclosed may be adapted as needed depending on the target enzyme properties desired.

Recombinant host cells comprising one or more heterologous nucleic acids encoding a malate-CoA ligase of the present disclosure demonstrate increased glycolic acid titers and/or yields in fermentation as compared to parental or control cells that do not comprise said heterologous nucleic acid(s). In some embodiments, recombinant host cells comprising one or more heterologous nucleic acids encoding a malate-CoA ligase produce an increased glycolic acid titer in fermentations as compared to parental or control cells that do not comprise said heterologous nucleic acid(s). In some embodiments, the glycolic acid titer is increased by 0.5 g/l, 1 g/l, 2.5 g/l, 5 g/l, 7.5 g/l, 10 g/l, or more than 10 g/l. In some embodiments, recombinant host cells comprising one or more heterologous nucleic acids encoding a malate-CoA ligase have increased glycolic acid yield as compared to parental or control cells that do not comprise said heterologous nucleic acid(s). In some embodiments, the glycolic acid yield is increased by 0.5%, 1%, 2.5%, 5%, 7.5%, 10%, or more than 10% (g-glycolic acid/g-substrate, or g-downstream product/g-substrate).

In some embodiments, a mutated malate-CoA ligase displays improved kinetic properties, such as Kₘ and k_{cat} when using malate as the substrate. In some embodiments, the malate-CoA ligase is a product of one or more protein engineering cycles. In some embodiments, the malate-CoA ligase comprises one or more point mutations. In some embodiments, a mutated malate-CoA ligase has improved Kₘ and/or k_{cat} for the substrate malate. In some embodiments, the malate-CoA ligase has Kₘ≤3 mM with malate as substrate. In some embodiments, the malate-CoA ligase has k_{cat}≥10 per second with malate as substrate.

Following strain library generation and screening, strain variants with desired evolved phenotype(s) are analyzed for malate-CoA ligase activity. In some embodiments, in vitro characterization assays are carried out as described below in section 2.3. In some embodiments, culture median or fermentation broth is analyzed for the presence of metabolites such as malate-CoA.

In many embodiments, the malate-CoA ligase is derived from a bacterial source. In many of these embodiments, the malate-CoA ligase is derived from a host cell belonging to a genus selected from the group comprising Methanothermobacter, Methylobacterium, Mezorhizobium, Roseobacter, Beauveria, Acetobacter, Rhodobacter, Methanosaeta, Salmonella, Zoogloea, Sinorhizobium, Escherichia, Advenella, Alcanivorax, and Nitrosomonas, Non-limiting examples of proteins from which malate-CoA ligase is derived from comprise Methylobacterium extorquens UniProt ID: P53594 and UniProt ID: P53595, Mezorhizobium japonicum UniProt ID: Q98KT8 and UniProt ID:Q98KT9, Roseobacter denitrificans UniProt ID: Q16B30 and UniProt ID:Q16B02, Advenella mimigardefordenis UniProt ID: W0PAN5 and UniProt ID:W0PFR9, Advenella mimigardefordensis UniProt ID: B3TZD8 and UniProt ID: B3TZD9; Acetobacter aceti UniProt ID: B3EY95, Alcanivorax borkunensis UniProt ID: Q0VPF7 and UniProt ID: Q0VPF8, and Escherichia coli UniProt ID: P0A36 and UniProt ID: P0AGE9, Methanosaeta thermophila UniProt ID: A0B8F, Methanothermobacter thermautotrophicus UniProt ID: Q210L7, Methanothermobacter thermautotrophicus UniProt ID: Q2XNL6, and Beauveria bassiana UniProt ID: A0A0A2VTS4. (Each example has 2 UniProt IDs because the protein candidates are heterodimers.)

In many embodiments, the malate-CoA ligase is derived from an archaeal source. In many of these embodiments, the malate-CoA ligase is derived from a host cell belonging to a genus selected from the group comprising Pyrococcus.

In many embodiments, the malate-CoA ligase is derived from a eukaryotic source. In many of these embodiments, the malate-CoA ligase is derived from a host cell belonging to a genus selected from the group comprising Pichia, Sacchraomyces, Pisum, Homo, Bos, Archaeoglobus, Oryctolagus, Ovis, Penicillium, Paecilomyces, Sus, Rattus, Spinacia, Glycine, and Columba. Non-limiting examples of proteins from which malate-CoA ligase is derived from comprise Penicillium chrysogenum UniProt ID: 074725, and Homo sapiens UniProt ID: Q9NR19.

In some embodiments, the malate-CoA ligase is derived from the Advenella mimigardefordensis heterodimer succinate-CoA ligase (abbv. AmSUCD; UniProt ID: W0PAN5; SEQ ID NO: 6; and abbv. AmSUCC; UniProt ID: W0PFR9; SEQ ID NO: 7). In some embodiments, the malate-CoA ligase is derived from the Alcanivorax borkumensis heterodimer succinate-CoA ligase (abbv. AbSUCC; UniProt ID: Q0VPF7; SEQ ID NO: 11, and abbv. AbSUCD; UniProt ID: Q0VPF8; SEQ ID NO: 12). In some embodiments, the malate-CoA ligase is derived from the Escherichia coli heterodimer succinate CoA ligase (abbv. EcSUCC; UniProt ID: P0A836; SEQ ID NO 13; and abbv. EcSUCD; UniProt ID: P0AGE9; SEQ ID NO: 14). In some embodiments, the malate-CoA ligase is derived from the Methylobacterium extorquens heterodimer malate-CoA ligase (abbv. MeMCSA; UniProt ID: P53594; SEQ ID NO: 15; and abbv. MeMCSB; UniProt ID: P53595, SEQ ID NO: 16). In some embodiments, the malate-CoA ligase is derived from the Mezorhizobium japonicum (abbv. MjSUCD; UniProt ID: Q98KT8; SEQ ID NO: 63; and abbv, MjMTKA; UniProt ID: Q98KT9; SEQ ID NO: 3). In some embodiments, the malate-CoA ligase is derived from the Roseobacter denitrificans (abbv. RdMTKA; UniProt ID: Q16B30; SEQ ID NO: 56) and (abbv, RdMTKB; UniProt ID: Q16B29; SEQ ID NO: 52).

In many embodiments, recombinant host cells comprise one or more heterologous nucleic acids encoding a malate-CoA ligase wherein said recombinant host cells are capable of producing glycolic acid, in various embodiments, proteins suitable for use in accordance with methods of the present disclosure have malate-CoA ligase activity and comprise an amino acid sequence with at least 60%, at least 70%, at least 80%, at least 90%, or at least 95% sequence homology with SEQ ID NO: 3, SEQ ID NO: 6. SEQ ID NO: 7, SEQ ID NO: 11, SEQ ID NO: 12, SEQ ID NO: 13, SEQ ID NO: 14, SEQ ID NO: 15, SEQ ID NO: 16, SEQ ID NO: 52, SEQ ID NO: 56, or SEQ ID NO: 63, or. In many embodiments, the recombinant host cell is a P. kudriavzevii strain.

### 2.2.4 Malyl-CoA Lyase

The malyl-CoA lyase (EC #4.1.3.24) described herein catalyzes the conversion of 1 molecule of malyl-CoA to 1 molecule of acetyl-CoA and 1 molecule of glyoxylate. Any enzyme is suitable for use in accordance with the present disclosure so long as the enzyme is capable of catalyzing said malyl-CoA lyase reaction. Practitioners skilled in the art will appreciate that malyl-CoA is synonymous with (3S)-3-carboxy-3-hydroxypropanoyl-CoA and these terms are used interchangeably in this disclosure. Similarly, malyl-CoA lyase and (3S)-3-carboxy-3-hydroxypropanoyl-CoA glyoxylate-lyase are synonyms and are used interchangeably in this disclosure.

Recombinant host cells comprising one or more heterologous nucleic acids encoding a malyl-CoA lyase of the present disclosure have an increase in glycolic acid titer and/or yield as compared to parental or control cells that do not comprise said heterologous nucleic acid(s). In some embodiments, recombinant host cells comprising one or more heterologous nucleic acids encoding a malyl-CoA lyase produce an increased glycolic acid titer in fermentations as compared to parental or control cells that do not comprise said heterologous nucleic acid(s). In some embodiments, the glycolic acid titer is increased by 0.5 g/l, 1 g/l, 2.5 g/l, 5 g/l, 7.5 g/l, 10 g/l, or more than 10 g/l. In some embodiments, recombinant host cells comprising one or more heterologous nucleic acids encoding a malyl-CoA lyase have increased glycolic acid yield as compared to parental or control cells that do not comprise said heterologous nucleic acid(s). In some embodiments, the glycolic acid yield is increased by 0.5%, 1%, 2.5%, 5%, 7.5%, 10%, or more than 10% (g-glycolic acid/g-substrate, or g-downstream product/g-substrate).

In some embodiments, the malyl-CoA lyase of the present disclosure is derived from a formimidoyltetrahydrofolate cyclodeaminase (EC #; 4.3.14).

In many embodiments, the malyl-CoA lyase is derived from a bacterial source. In many of these embodiments, the malyl-CoA lyase is derived from a host cell belonging to a genus selected from the group comprising Pseudomonas, Rhodobacter, Chloroflexus, Methylobacterium, Roseobacter, Roseovarious, and Magnetospira, Non-limiting examples of bacterial enzymes comprise Rhodobacter sphaeroides UniProt ID: Q3J5L6, Rhodobacter capsulatus UniProt ID: D5AR83, Chloroflexus aurantiacus UniProt ID: S5N020, Methylpyrrolium extorque UniProt ID: P71503, Rhodobacter sp. UniProt ID: V7FJP0, Roseobacter sp. UniProt ID: A3XFN7, Roseovarius nubinhibens UniProt ID: A3SLS9, and Magnetospira sp. UniProt ID: W6K7J1.

In many embodiments, the malyl-CoA lyase is derived from a eukaryotic source. In many of these embodiments, the malyl-CoA lyase is derived from a host cell belonging to a genus selected from the group comprising Anas, Homo, and Raphanus.

In some embodiments, the malyl-CoA lyase is derived from the Rhodobacter sphaeroides L-malyl-CoA/beta-methylamyl-CoA lyase (abbv. RsMCL1; UniProt ID: Q3J5L6; SEQ ID NO: 23). In some embodiments, the malyl-CoA lyase is derived from the Rhodobacter capsulatus L-malyl-CoA/beta-methylamyl-CoA lyase (abbv. RcMCL1; UniProt ID: D5AR84; SEQ ID NO: 24). In some embodiments, the malyl-CoA lyase is derived from the Chloroflexus aurantiacus malyl-CoA/beta-methylamyl-CoA/citramalyl-CoA lyase (abbv. CaMCL; UniProt ID: S5N020; SEQ ID NO: 25). In some embodiments, the malyl-CoA lyase is derived from the Methylobacterium extorquens (malyl-)CoA ester lyase (abbv, MeMCLA; UniProt ID: P71503; SEQ ID NO: 26). In some embodiments, the malyl-CoA lyase is derived from the Rhodobacter sp, malyl-CoA lyase (abbv. RsMCL; UniProt ID: V7EJP0; SEQ ID NO: 27). In some embodiments, the malyl-CoA lyase is derived from the Roseobacter sp, malyl-CoA lyase (abbv. RsMCL2; UniProt ID: A3XFN7; SEQ ID NO: 28). In some embodiments, the malyl-CoA lyase is derived from the Roseovarius nubinhibens lyase malyl-CoA lyase (abbv. RnMCL; UniProt ID: A3SLS9; SEQ ID NO: 29). In some embodiments, the malyl-CoA lyse is derived from the Magnetospira sp, malyl-CoA lyase (abbv. MsMCL; UniProt ID: W6K7J1; SEQ ID NO: 30).

In many embodiments, recombinant host cells comprise one or more heterologous nucleic acids encoding a malyl-CoA lyase wherein said recombinant host cells are capable of producing glycolic acid, in various embodiments, proteins suitable for use in accordance with methods of the present disclosure have malyl-CoA lyase activity and comprise an amino acid sequence with at least 40%, at least 50%, at least 60%, at least 70%, at least 80%, at least 90%, or at least 95% sequence homology with SEQ ID NO: 23, SEQ ID NO: 24, SEQ ID NO: 25, SEQ ID NO: 26, SEQ ID NO: 27. SEQ ID NO: 28, SEQ ID NO: 29, or SEQ ID NO: 30. In many embodiments, the recombinant host cell is a P. kudriavzevii strain.

The malyl-CoA lyase consensus sequence #1 (SEQ ID NO: 21) provides the sequence of amino acids in which each position identifies the amino acid (if a specific amino acid is identified) or a subset of amino acids (if a position is identified as variable) most likely to be found at a specific position in a malyl-CoA lyase. Many amino acids in consensus sequence #1 (SEQ ID NO: 21) are highly conserved and malyl-CoA lyases suitable for use in accordance with the methods of the present disclosure will comprise a substantial number, and sometimes all, of these highly conserved amino acids at positions aligning with the location of the indicated amino acid in consensus sequence #1 (SEQ ID NO: 21). In various embodiments, proteins suitable for use in accordance with the methods of the present disclosure have malyl-CoA lyase activity and comprise an amino acid sequence with at least 40%, at least 50%, at least 60%, at least 65%, or at least 70% sequence homology with consensus sequence ##1 (SEQ ID NO: 21). For example, the RsMCL1 sequence (SEQ ID NO: 23) is at least 89% homologous to consensus sequence #1 (SEQ ID NO: 21) and is therefore encompassed by consensus sequence #1 (SEQ ID NO: 21), in another example, the CaMCL sequence (SEQ ID NO: 25) is at least 44% homologous to consensus sequence #1 (SEQ ID NO: 21) and is therefore encompassed by consensus sequence #1 (SEQ ID NO: 21). In another example, the McMCL sequence (SEQ ID NO: 30) is at least 45% homologous to consensus sequence #1 (SEQ ID NO: 21) and is therefore encompassed by consensus sequence #1 (SEQ ID NO: 21). In another example, the MeMCLA sequence (SEQ ID NO: 26) is at least 60% homologous to consensus sequence #1 (SEQ ID NO: 21) and is therefore encompassed by consensus sequence #1 (SEQ ID NO: 21). In another example, the RnMCL sequence (SEQ ID NO: 29) is at least 88% homologous to consensus sequence #1 (SEQ ID NO: 21) and is therefore encompassed by consensus sequence #1 (SEQ ID NO: 21). In another example, the RsMCL2 sequence (SEQ ID NO: 28) is at least 87% homologous to consensus sequence #1 (SEQ ID: NO: 21) and is therefore encompassed by consensus sequence #1 (SEQ ID NO: 21). In another example, the RsMCL sequence (SEQ ID NO: 27) is at least 88% homologous to consensus sequence #1 (SEQ ID NO. 21) and is therefore encompassed by consensus sequence #1 (SEQ ID NO: 21).

Highly conserved amino acids in consensus sequence #1 (SEQ ID NO: 21) are R15, P21, A33, D37, V38, L43, E44, D45, K52, A55, R56, I60, G69, R76, N78, L80, D88, L100, D101, P106, K107, V108, D113, D118, E125, E141, A143, G145, I152, A153, S156, R158, S163, G165, A167, D168, A170, A171, S172, T178, GI81, GI82, Y187, D201. W203, G218, G224, P225, F226, G227, D228, D231, R240, L245, G246, G249, W251, P255, Q257, A261, V264, F265, P267. V272, A275, I278, L279, A281, M282, G289, G291, G297, D301, A303, and A308, Enzymes homologous to SEQ ID NO: 21 will contain a majority of these conserved amino acids at positions aligning with (i.e., corresponding to) the highly conserved amino acids in SEQ ID NO: 21. In various embodiments, malyl-CoA lyases homologous to SEQ ID NO: 21 comprise at least 40%, at least 50%, at least 60%, at least 70%, at least 80%, at least 85%, at least 90%, at least 95%, or sometimes all of these highly conserved amino acids at positions corresponding to the highly conserved amino acids identified in SEQ ID NO: 21. In some embodiments, each of these highly conserved amino acids are found in a desired malyl-CoA lyases as provided, for example, in SEQ ID NO: 21.

### 2.2.5 Glyoxylate Reductase

The glyoxylate reductase (EC #1.1.1.26) described herein catalyzes the conversion of 1 molecule of glyoxylate and 1 molecule of NAD(P)H to 1 molecule of glycolate and 1 molecule of NAD(P)⁺. Any enzyme is suitable for use in accordance with the present disclosure so long as the enzyme is capable of catalyzing said glyoxylate reductase reaction. Practitioners skilled in the an will appreciate that glyoxylate reductase is synonymous with glyoxylic acid reductase and these terms are used interchangeably in this disclosure. In some embodiments, the glyoxylate reductase preferentially utilizes NADH instead of NADPH.

Recombinant host cells comprising one or more heterologous nucleic acids encoding a glyoxylate reductase of the present disclosure have an increase in glycolic acid titer and/or yield as compared to parental or control cells that do not comprise said heterologous nucleic acid(s). In some embodiments, recombinant host cells comprising one or more heterologous nucleic acids encoding a glyoxylate reductase produce an increased glycolic acid titer in fermentations as compared to parental or control cells that do not comprise said heterologous nucleic acid(s). In some embodiments, the glycolic acid titer is increased by 0.5 g/l, 1 g/l, 2.5 g/l, 5 g/l, 7.5 g/l, 10 g/l, or more than 10 g/l. In some embodiments, recombinant host cells comprising one or more heterologous nucleic acids encoding a glyoxylate reductase have increased glycolic acid yield as compared to parental or control cells that do not comprise said heterologous nucleic acid(s). In some embodiments, the glycolic acid yield is increased by 0.5%, 1%, 2.5%, 5%, 7.5%, 10%, or more than 10% (g-glycolic acid/g-substrate, or g-downstream product/g-substrate).

In some embodiments, the glyoxylate reductase of the present disclosure is derived from a D-lactate dehydrogenase (EC #1.1.128). In some embodiments, the glyoxylate reductase of the present disclosure is derived from a L-lactate dehydrogenase (EC #1.1.1.27). In some embodiments, the glyoxylate reductase of the present disclosure is derived from a diacetyl reductase [(S)-acetoin forming] (EC #L1.1.304) In some embodiments, the glyoxylate reductase of the present disclosure is derived from a glycolate dehydrogenase (EC #1.1.99.14).

In many embodiments, the glyoxylate reductase is derived from a bacterial source. In many of these embodiments, the glyoxylate reductase is derived from a host cell belonging to a genus selected from the group comprising Thermococcus, Rhizobium, Pediococcus, Lactobacillus, Leuconostoc, Staphylococcus, Enterococcus, Nocardia, Thermotogam, Geobacillus, Thermus, Thermoanaerobacter, Mycobacterium, Klebsiella, Bacillus, Enterobacter, Lactococcus, Streptococcus, and Dienococcus. Non-limiting examples bacterial enzymes comprise Thermococcus litoralis UniProt ID: Q9C4M5, Leuconostoc mesenteroides subsp. cremoris UniProt ID: P51011. Geobacillus stearothermophilus UniProt ID: P00344, Thermus thermophiles UniProt ID: Q5SJA1, Thermoanaerobacter methanolicus UniProt ID: B1A4R6, Escherichia coli UniProt ID: P75913, Klebsiella pneumoniae UniProt ID: B5XMZ4, Mycobacterium sp. UniProt ID: W8VSK8, and Deinococcus radiodurans UniProt ID: P50933.

In many embodiments, the glyoxylate reductase is derived from an archaeal source. In many of these embodiments, the glyoxylate reductase is derived from a host cell belonging to the genus Sulfolobus. A non-limiting example of an archaeal enzyme is Sulfolobus solfataricus UniProt ID: Q97U35.

In many embodiments, the glyoxylate reductase is derived from a eukaryotic source. In many of these embodiments, the glyoxylate reductase is derived from a host cell belonging to a genus selected from the group comprising Saccharomyces, Arabidopsis, Spinacia, Homo, Limulus, Haliotis, Helix, Epidalea, Sphyraena, Plasmodium, Agama, Cryptosporidium, Columba, Pelophylax, Clupea, Saduria, Hoplodactylus, Oligosoma, Cyclodina, Sus, Champsocephalus, Solanum, Homarus, Capsella, Ipomoea, Molinema, Rhizopus, Ipomoea, Taenia, and Lactuca. Non-limiting examples eukaryotic enzymes comprise Saccharomyces cerevisiae UniProt ID: P53839, Plasmodium vivax UniProt ID: Q6JH30, Cryptosporidium parvum UniProt ID: Q9GT92, Plasmodium falciparum UniProt ID: Q27743, Champsocephalus gunnari UniProt ID: Q93541, Sphyraena hucasana UniProt 23 ID: O13278, Sphyraena idiastes UniProt ID: 013277, and Sphyraena argentea UniProt ID: O13276. In some embodiments, the glyoxylate reductase is derived from the Saccharomyces cerevisiae g/lyoxylate reductase (abbv. ScGOR1; UniProt ID: P53839; SEQ ID NO: 31). In some embodiments, the glyoxylate reductase is derived from the Sulfolobus solfataricus SERA-2 (abbv. SsSERA-2; UniProt ID: Q97U35; SEQ ID NO: 32). In some embodiments, the glyoxylate reductase is derived from the Escherichia coli g/lyoxylate/hydroxypyruvate reductase A (abbv. EcGHRA; UniProt ID: P75913; SEQ ID NO: 33). In some embodiments, the glyoxylate reductase is derived from the Klebsiella pneumoniae glyoxylate/hydroxypyruvate reductase B (abbv. KpGHRB; UniProt ID: B5XMZ4; SEQ ID NO: 34). In some embodiments, the glyoxylate reductase is derived from the Escherichia coli glyoxylate/hydroxypyruvate reductase A (abbv. EcGHRA2; UniProt ID: S0WM29; SEQ 10 NO: 5). In some embodiments, the glyoxylate reductase is derived from the Escherichia coli glyoxylate/hydroxypyruvate reductase B (abbv. EcGHRB; UniProt ID: Q1R343; SEQ ID NO: 80): In some embodiments, the glyoxylate reductase is derived from the Escherichia fergusonii g/lyoxylate/hydroxypyruvate reductase B (abbv. EfGHRB; UniProt ID: B7LTG7; SEQ ID NO: 81). In some embodiments, the glyoxylate reductase is derived from the Kluyvera georgiana g/lyoxylate/hydroxypyruvate reductase B (abbv. KgGHRB; UniProt ID: A0A248KG17; SEQ ID NO: 82). In some embodiments, the glyoxylate reductase is derived from the Citrobacter koseri g/lyoxylate/hydroxypyruvate reductase B (abbv. CkGHRB; UniProt ID: A8ARD9; SEQ ID NO: 83). In some embodiments, the glyoxylate reductase is derived from the Salmonella sp. HMSC13B08 g/lyoxylate/hydroxypyruvate reductase B (abbv. SsGHRB; UniProt ID: A0AIF2JL17; SEQ ID NO: 84). In some embodiments, the glyoxylate reductase is derived from the Escherichia coli SEQ SC6 g/lyoxylate/hydroxypyruvate reductase B (abbv. EcGHRB2; UniProt ID: W1H84; SEQ ID NO: 66). In some embodiments, the glyoxylate reductase is derived from the Klebsiella pneumoniae g/lyoxylate reductase (abbv. KpGLYR; UniProt ID: B5XMZ4; SEQ ID NO: 17). In some embodiments, the glyoxylate reductase is derived from the Sulfolobus solfataricus g/lyoxylate reductase (abbv. SsGLYR; UniProt ID: Q97U35; SEQ ID NO: 18), Arabidopsis thaliana g/lyoxylate reductase (abbv. AtGLYR; UniProt ID: Q9LSV0; SEQ ID NO: 19). In some embodiments, the glyoxylate reductase is derived from the Rhizobium etli g/lyoxylate reductase (abbv. ReGLYR; UniProt ID: C1 JH53, SEQ ID NO: 20)

In many embodiments, recombinant host cells comprise one or more heterologous nucleic acids encoding a glyoxylate reductase wherein said recombinant host cells are capable of producing glycolic acid, in various embodiments, proteins suitable for use in accordance with methods of the present disclosure have glyoxylate reductase activity and comprise an amino acid sequence with at least 60%, at least 70%, at least 80%, at least 90%, or at least 95% sequence homology with SEQ ID NO: 5, SEQ ID NO: 17, SEQ ID NO: 18. SEQ ID NO: 19, SEQ ID NO: 20, SEQ ID NO: 31. SEQ ID NO: 32, SEQ ID NO: 33. SEQ ID NO: 34, SEQ ID NO: 80. SEQ ID NO: 81, SEQ ID NO: 82. SEQ ID NO: 83, SEQ ID NO: 84, or SEQ ID NO: 66. In many embodiments, the recombinant host cell is a P. kudriavzevii strain.

The glyoxylate reductase consensus sequence #2 (SEQ ID NO: 22) provides the sequence of amino acids in which each position identifies the amino acid (if a specific amino acid is identified) or a subset of amino acids (if a position is identified as variable) most likely to be found at a specific position in a glyoxylate reductase. Many amino acids in consensus sequence #2 (SEQ ID NO: 22) are highly conserved and glyoxylate reductases suitable for use in accordance with the methods of the present disclosure will comprise a substantial number, and sometimes all, of these highly conserved amino acids at positions aligning with the location of the indicated amino acids in consensus sequence #2 (SEQ ID NO: 22). In various embodiments, proteins suitable for use in accordance with the methods of the present disclosure have glyoxylate reductase activity and comprise an amino acid sequence with at least 60%, at least 65%, or at least 70′% sequence homology with consensus sequence #2 (SEQ ID NO:22). For example, the EcGHRB (SEQ ID NO: 80) sequence is at least 89% homologous to consensus sequence 42 (SEQ ID NO: 22), and is therefore encompassed by consensus sequence #2 (SEQ ID NO: 22), in another example, the KgGHRB (SEQ ID NO: 82) sequence is at least 91% homologous to consensus sequence #2 (SEQ ID NO: 22), and is therefore encompassed by consensus sequence #2 (SEQ ID NO: 22). In another example, the EfGHRB (SEQ ID NO: 81) sequence is at least 91% homologous to consensus sequence #2 (SEQ ID NO: 22), and is therefore encompassed by consensus sequence #2 (SEQ ID NO: 22). In another example, the KpGHRB (SEQ ID NO: 34) sequence is at least 93% homologous to consensus sequence #2 (SEQ ID NO: 22), and is therefore encompassed by consensus sequence #2 (SEQ ID NO: 22).

In another example, the EcGHRB (SEQ ID NO: 66) sequence is at least 94% homologous to consensus sequence, #2 (SEQ ID NO: 22), and is therefore encompassed by consensus sequence #2 (SEQ ID NO: 22). In another example, the CkGHRB (SEQ ID NO: 83) sequence is at least 95% homologous to consensus sequence #2 (SEQ ID NO: 22), and is therefore encompassed by consensus sequence #2 (SEQ ID NO: 22). In another example, the SsGHRB (SEQ ID NO: 84) sequence is at least 95% homologous to consensus sequence #2 (SEQ ID NO: 22), and is therefore encompassed by consensus sequence #2 (SEQ ID NO: 22).

Highly conserved amino acids in consensus sequence #2 (SEQ ID NO: 22) are 16, Y8, D13, P33, G51, A59, L60, P65, L67, A69, G76, D78, L85, M93, P96, E101, L112, R116, R117, W130, P136, T147, G149, I150, G152, G154, G157, A161, Q162, F168, F187, L194, L198, L208, P209, E213, T214, A230, N234, R237, G238, V241, E243, L246, A249, L250, G253, A258, L260, D261, V262, P263, E266, P267, L268, S272, P273, L274, V280, P284, H285, T290, I306, and L309, Enzymes homologous to SEQ ID NO: 22 will contain a majority of these conserved amino acids at position aligning with (i.e., corresponding to) the highly conserved amino acids in SEQ ID NO: 22. In various embodiments, glyoxylate reductases homologous to SEQ ID NO: 22 comprise at least 60%, at least 70% at least 80%, at least 85%, at least 90%, at least 95%, or sometimes all of these highly conserved amino acids at positions corresponding to the highly conserved amino acids identified in SEQ ID NO: 22. In some embodiments, each of these highly conserved amino acids are found in a desired glyoxylate reductases as provided, for example, in SEQ ID NO: 22.

Due to sequence diversity in glyoxylate reductases, certain proteins will have glyoxylate reductase activity suitable for use in accordance with the present disclosure but may not be encompassed by consensus sequence #2 (SEQ ID NO: 22). In some embodiments, these proteins may share some, most, or all of the highly conserved amino acids in consensus sequence #2, which give rise to the glyoxylate reductase activity.

2.3 Methods to Identify and/or Improve Enzymes in the Glycolic Add Pathway

The present disclosure provides methods for the construction and characterization of malate-CoA ligase. The following exemplary methods have been developed for mutagenesis and diversification of genes for engineering specific or enhanced properties of targeted enzymes. Practitioners in the an will appreciate that the methods disclosed may be adapted as needed depending on the target enzyme properties desired. In some instances, the disclosed methods are suitable for use in engineering enzymes towards malate-CoA ligase of the glycolic acid pathway. In some embodiments, the malate-CoA ligase is derived from an enzyme with native activity towards a substrate that is structurally similar to malate-CoA. In many embodiments, the malate-CoA ligase is derived from an acid thiol ligase (EC #6.2.1.X), which comprises malate-CoA ligase (EC #6.2.1.9), succinate-CoA ligase (EC #6.2.1.5 or EC #6.2.1.4), acetate-CoA ligase (EC #6.2.1.1), butyrate-CoA ligase (EC #6.2.1.2), and/or an acetoacetate-CoA ligase (EC #6.2.1.16).

Methods described herein for protein mutagenesis, identification, expression, purification, and characterization are methods widely-practiced by practitioners skilled in the art, who will appreciate that a wide variety of commercial solutions are available for such endeavors. Practitioners will understand that identification of mutated proteins comprise activity screens and phenotypic selections.

### 2.3.1 Generating Protein Libraries Via Mutagenesis

Enzymes that are identified as good mutagenesis starting points enter the protein engineering cycle, which comprises protein mutagenesis, protein identification, protein expression, protein characterization, recombinant host cell characterization, and any combination thereof. Iterative rounds of protein engineering are typically performed to produce an enzyme variant with properties that are different from the template/original protein. The enzyme variants of the present disclosure comprise malate-CoA ligase activity, Examples of enzyme characteristics that are improved and/or altered by protein engineering include, for example, substrate binding (Kₘ; i.e., a measure of enzyme binding affinity for a particular substrate) that includes non-natural substrate selectivity/specificity: enzymatic reaction rates (k_{cat}, the turnover rate of a particular enzyme-substrate complex into product and enzyme), to achieve desired pathway flux; temperature stability, for high temperature processing pH stability, for processing in pH ranges outside the wild type protein pH range; substrate or product tolerance, to enable high product titers; removal of inhibition by products, substrates or intermediates; expression levels, to increase protein yields and overall pathway flux; oxygen stability, for operation of oxygen sensitive enzymes under aerobic conditions; and anaerobic activity, for operation of an aerobic enzyme in the absence of oxygen. In some embodiments, the enzyme variant enables improved glycolic acid pathway flux. In some embodiments, the enzyme variant enables increased glycolate yield, titer and/or productivity. In some embodiments, the enzyme variant enables increased substrate specificity. In some embodiments, the enzyme variant displays improved kinetic properties, such as decreased Kₘ and/or increased k_{cat}. In some embodiments, the enzyme variant has improved Kₘ and/or kk_{cat} for the substrates malate and CoA. In some embodiments, the enzyme variant has Kₘ≤3 mM with malate, CoA, and ATP as substrates. In some embodiments, the enzyme variant has k_{cat}≥10 turnovers per second with malate, CoA, and ATP as substrates. In sone embodiments, the enzyme variant is a product of one or more protein engineering cycles. In some embodiments, the enzyme variant comprises one or more point mutations.

In general, random and rational mutagenesis approaches are acceptable methods for generating DNA libraries of mutant proteins. Error-prone PCR is a random mutagenesis method widely used for generating diversity in protein engineering, and practitioners skilled in the art will recognize that error-prone PCR is not only fast and easy, but it is also a method that has successfully produced mutated enzymes with altered activity from a wild type DNA template, (Wilson, D. S. & Keefe, A. D. Random mutagenesis by PCR, Curr. Protoc. Mol. Biol. Chapter 8, Unit 8.3 (2001.) To help increase the odds of identifying an enzyme with malate-CoA ligase activity, rational mutagenesis of a small number of active site mutations is also useful. For example, in embodiments wherein the starting protein for malate-CoA ligase engineering is the succinate-CoA ligase, atomic structures of succinate-CoA ligase homologs are available to guide structural modeling of succinate-CoA ligase with malate and CoA bound in the active site. Practitioners in the art will appreciate that structural modeling allows one to identify amino acids in the active site believed to be important for substrate recognition. Other mutagenesis approaches that could be used include DNA shuffling and combinatorial mutagenesis. In some embodiments, the mutagenesis step is carried out more than once, resulting in iterative rounds of engineering.

Following library generation, mutated genes are typically cloned for expression in a host organism and in many cases the proteins are subsequently purified for in vitro activity screening. In some embodiments, the host organism is E. coli. Mutated genes are cloned in a suitable expression plasmid comprising an auto-inducible promoter upstream of the gene and a His-ag sequence downstream of the gene. The proteins expressed from such a plasmid are isolated from whole cell lysate with Ni-NTA affinity purification methods, such as the Takara Capturem His-tagged Purification 9. Purified proteins then enter an in vitro activity screen for characterization. The Ni-NTA affinity purification step helps to reduce background noise in in vitro activity screen.

### 2.3.2 Generating Strain Libraries Via Directed Evolution

In another aspect, directed evolution methods are used to identify enzymes with malate-CoA ligase activity and/or improved kinetic parameters (for example, decreasing the enzyme Kₘ and/or increasing the enzyme k_{cat} when using malate, CoA, and ATP as the substrates) of enzymes exhibiting low activity toward malate as a substrate, Directed evolution approaches are useful in generating strain libraries with a wide diversity of mutations wherein the mutations are driven by the process of natural selection given the constraints provided to the organism in its growth environment, Evolution approaches provide an effective and impartial way of introducing sequence mutations that give rise to functional change at an organism scale, enabling practitioners to explore non-intuitive mutations in the universe of possibilities that lie beyond the confines of one's understanding about structure-function specificity.

In some embodiments, a screen is designed to monitor the progress of evolution over time. In some of these embodiments, it is useful to link desired mutagenesis with a measurable phenotype so that the rate of evolution can be monitored over an extended period of time. In some of these embodiments, the measurable phenotype comprises cell growth, glucose consumption, and metabolite production. In some embodiments, the measurable phenotype is favored by a selection. In some embodiments, the directed evolution experiment is designed so that mutations acquired in the target gene(s) is a measurable phenotype that is advantageous to the organism. In some of these embodiments, the advantageous measurable phenotype comprises cellular fitness, energy production, growth rate, tolerance to toxicity, and tolerance to extreme culture conditions (such as high or low pH, high or low temperature, high or low osmotic pressure, drought, and nutrient limitation). In various embodiments, one or more synthetic metabolic pathways are constructed by introducing exogenous nucleic acids to recombinant host cells. In these embodiments, the one or more synthetic metabolic pathways provide a method of applying selective pressure or a method of selecting strain variants that result from directed evolution.

Besides a well-crafted screen and/or selection before the evolution experiment begins, starting nucleic acid templates for proteins of interest (i.e., target gene(s) or parent gene(s)) are also identified. In embodiments of the present disclosure, enzymes that serve as a good starting point for malate-CoA ligase engineering are identified. In these embodiments, nucleic acids that encode the template for malate-CoA ligase engineering are integrated into the genome of recombinant host cells. In some embodiments, the malate-CoA ligase is derived from an enzyme with native activity towards substrates that is structurally similar to malate. In some embodiments, the malate-CoA ligase is derived from an acid thiol ligase (EC #6.2.1.X), which comprises malate-CoA ligase (EC #6.2.1.9), succinate-CoA ligase (EC #6.2.1.4 or EC #6.2.1.5), acetate-CoA ligase (EC #6.2.1.1), butyrate-CoA ligase (EC #6.2.1.2), and acetoacetate-CoA ligase (EC #6.2.1.16).

Once a screen and/or selection is established and target genes (i.e., malate-CoA ligase, according to embodiments of the present disclosure) are identified and integrated into the genome of recombinant host cells, recombinant host cells enter the directed evolution cycle, wherein the directed evolution cycle comprises: (1) mutagenesis in response to selective pressure: (2) analysis of recombinant host cells in the generated library for measurable phenotypic differences that arise due to selective pressure; and (3) isolation and characterization of evolved variants.

In some embodiments, acquisition of a mutation in the target gene enables the recombinant host cell to overcome the selective pressure. In some embodiments, recombinant host cells are passaged throughout the course of mutagenesis with selective pressure. In various embodiments, the selective pressure comprises nutrient limitation, cellular toxicity, and extreme culture conditions that further comprise high or low pH, high or low temperature, and high or low osmotic pressure. In some embodiments, the recombinant host cells are initially propagated without selective pressure prior to mutagenesis.

After exposure to selective pressure for some period of time, the evolved or evolving strains are screened for a change in phenotype in response to selective pressure. Non-limiting examples of phenotypic change include faster glucose consumption, faster cell growth, higher flux through a metabolic pathway or pathways, improved product yield/titer/productivity, decreased byproduct yield/titer, increased tolerance to toxicity, or increased tolerance to extreme culture conditions.

### 2.3.3 Enzyme Characterization

Following protein library generation, protein variants can be screened for malate-CoA ligase activity. With malate. CoA, and ATP as substrates, for example, kinetic parameters Kₘ (i.e., binding affinity for a substrate) and k_{cat} (i.e., turnover rate of an enzyme-substrate complex into product and enzyme) are calculated from an in vitro activity assay, After identifying malate-CoA ligase with this initial assay, the same assay can be used to test malate-CoA ligase substrate specificity, in addition to malate, CoA, and ATP, a variety of substrates can be provided as substrates in the assay to determine if the engineered malate-CoA ligase can react with other substrates, leading to byproducts that could be problematic in a commercial process.

Protein variants that result from strain library generation and screening can also be analyzed for malate-CoA ligase activity, Strain variants with desired evolved phenotype(s) are typically isolated and characterized, in some embodiments, mutations are acquired by the nucleic acids encoding target proteins. In these embodiments, nucleic acids encoding target proteins are sequenced so that acquired mutations are identified, in other embodiments, mutations are acquired by nucleic acids that are native to the recombinant host cells. In some embodiments, target proteins are analyzed for malate-CoA ligase activity.

In some embodiments, iterative rounds of protein engineering are performed to produce enzyme variants with optimized properties, wherein the iterative rounds of protein engineering comprise rational mutagenesis, random mutagenesis, and directed evolution. In these embodiments, select variants from preceding rounds of protein engineering are identified for further protein engineering. Non-limiting examples of such properties comprise improved enzyme kinetics for specificity and/or turnover, improved pathway flux, increased metabolite yield, decreased byproduct yield. In some embodiments, culture medium or fermentation broth is analyzed for the presence of metabolites such as glycolic acid and/or byproducts, wherein the method of analysis is HPLC (high-performance liquid chromatography).

### 2.4 Ancillary Proteins

In addition to the glycolic acid pathway enzymes and/or downstream product pathway enzymes, ancillary proteins are other proteins that are overexpressed in recombinant host cells of the present disclosure whose overexpression results in an increase in glycolic acid and/or downstream product yields, productivities, and/or titers as compared to control, or host cells that do not overexpress said proteins. Ancillary proteins function outside the glycolic acid pathway and/or the downstream product pathway, wherein each ancillary protein plays a role that boosts the recombinant host cell's ability to produce glycolic acid and/or downstream product. Ancillary proteins comprise any protein (excluding glycolic acid pathway enzymes and downstream product pathway enzymes) ofany structure or function that can increase glycolic acid and/or downstream product yields, titers, or productivities when overexpressed. Non-limiting examples of classes of proteins include transcription factors, transporters, scaffold proteins, proteins that decrease byproduct accumulation, and proteins that regenerate or synthesize redox cofactors.

Provided herein in certain embodiments are recombinant host cells comprising one or more heterologous nucleic acids encoding one or more ancillary proteins wherein said recombinant host cell is capable of producing higher glycolic acid and/or downstream product yields, titers, or productivities as compared to control cells, or host cells that do not comprise said heterologous nucleic acid(s). In some embodiments, that host recombinant cell naturally produces glycolic acid and/or downstream product, and in these cases, the glycolic acid and/or downstream product yields, liters, and/or productivities are increased. In other embodiments, the recombinant host cell comprises one or more heterologous nucleic acids encoding one or more glycolic acid pathway enzymes and/or downstream product pathway enzymes.

In certain embodiments of the present disclosure, the recombinant host cells comprise one or more heterologous nucleic acids encoding one or more glycolic acid pathway enzymes and/or one or more downstream product pathway enzymes, and one or more heterologous nucleic acids encoding one or more ancillary proteins. In certain of these embodiments, the recombinant host cells may be engineered to express more of these ancillary proteins. In these particular embodiments, the ancillary proteins are expressed at a higher level (i.e., produced at a higher amount as compared to cells that do not express said ancillary proteins) and may be operatively linked to one or more exogenous promoters or other regulatory elements.

In certain embodiments, recombinant host cells comprise both endogenous and heterologous nucleic acids encoding one or more glycolic acid pathway enzymes and/or one or more downstream product pathway enzymes and one or more ancillary proteins. In certain embodiments, the recombinant host cells comprise one or more heterologous nucleic acids encoding one or more glycolic acid pathway enzymes and/or one or more ancillary proteins, and one or more endogenous nucleic acids encoding one or more glycolic acid pathway enzymes and/or one or more downstream product pathway enzymes, and/or one or more ancillary proteins.

In certain embodiments, endogenous nucleic acids of ancillary proteins are modified in situ (i.e., on chromosome in the host cell genome) to alter levels of expression, activity, or specificity. In some embodiments, heterologous nucleic acids are inserted into endogenous nucleic acids of ancillary proteins.

### 2.4.1 Ancillary Proteins for Redox Cofactor Recycling

One type of ancillary proteins is proteins that recycle the redox cofactors that are produced during glycolic acid pathway activity. Redox balance is fundamental to sustained metabolism and cellular growth in living organisms. Intracellular redox potential is determined by redox cofactors that facilitate the transfer of electrons from one molecule to another within a cell. Redox cofactors in yeast include the nicotinamide adenine dinucleotides, NAD and NADP, the flavin nucleotides. FAD and FMN, and iron sulfur clusters (Fe—S clusters).

Redox constraints play an important role in end-product formation. Additional reducing power must be provided to produce compounds whose degree of reduction is higher than that of the substrate. For example, the glycolic acid pathway of the present disclosure results in the oxidation of 2 molecules of NAD(P)H to 2 molecules of NAD(P)⁺ for every molecule of glucose that is converted to 2 molecules of glycolic acid. Reduction of NAD(P)⁺ back to NAD(P)H is important for maintaining the thermodynamic diving force necessary for efficient and rapid glycolic acid production. This means that other processes in the cell must operate to restore the redox imbalance caused; for example, NAD(P)H can be generated from acetyl-CoA catabolism. In certain embodiments, the ancillary proteins are expressed in the cytosol of recombinant host cells to provide said additional reducing power or to restore redox balance. In certain embodiments, the ancillary proteins are associated with the mitochondrial or cell membrane of the recombinant host cells.

### 2.4.2 Ancillary Proteins for Redox Cofactor Biogenesis

As explained in preceding paragraphs, redox balance is crucial for cell growth and sustained metabolism. Two out of the five glycolic acid pathway enzymes utilize redox cofactors that must be generated, in addition to being recycled, for robust metabolism and cell vitality. In some embodiments of the present disclosure, recombinant host cells comprise a malate dehydrogenase that utilizes NAD(P)H. In some embodiments, recombinant host cells comprise a glyoxylate reductase that utilizes NAD(P)H. Thus, biogenesis and homeostasis of these cofactors are crucial for efficient catalysis of these enzymatic reactions.

The NAD and NADP cofactors are involved in electron transfer and contribute to approximately 12% of all biochemical reactions in a cell (Osterman A., EcoSal Plus, 2009). NAD is assembled from L-aspartate, dihydroxyacetone phosphate (DHAP; glycerone), phosphoribosyl pyrophosphate (PRPP) and ATP. NA DP is assembled in the same manner and further phosphorylated. In some embodiments, recombinant host cells comprise heterologous and/or endogenous nucleic acids encoding one or more ancillary proteins that facilitate NAD and NADP cofactor assembly. In some embodiments, the ancillary proteins comprise one, more than one, or all proteins suitable for use in accordance with methods of the present disclosure having NAD and/or NADP assembly capability. NAD and/or NADP transfer capability, NAD and/or NADP chaperone capability, or any combination thereof.

Similarly, Fe—S clusters facilitate various enzyme activities that require electron transfer. Because both iron and sulfur atoms are highly reactive and toxic to cells, Fe—S cluster assembly requires carefully coordinated synthetic pathways in living cells, Three known pathways are the Isc (iron sulfur cluster) system, the Suf (sulfur formation) system, and the Nif (nitrogen fixation) system. Each of these systems has a unique physiological role, yet several functional components are shared between them. First, a cysteine desulfurase enzyme liberates sulfur atoms from free cysteine. Then, a scaffold protein receives the liberated sulfur for Fe—S cluster assembly. Finally, the Fe—S cluster is transferred to a target apoprotein. In some embodiments of the present disclosure, recombinant host cells comprise heterologous and/or endogenous nucleic acids encoding one or more ancillary proteins that facilitate Fe—S cluster assembly. In some embodiments, the ancillary proteins comprise one, more than one, a plurality or all proteins of the Isc system, the Suf system, the Nif system, or any combination thereof. In some embodiments, recombinant host cells comprise one or more heterologous nucleic acids encoding one or more proteins suitable for use in accordance with methods of the present disclosure having cysteine desulfurase activity, Fe—S cluster assembly capability, Fe—S cluster transfer capability, iron chaperone capability, or any combination thereof,

2.4.3 Ancillary Proteins for Glycolic Acid and/or Downstream Product Transport

Another class of ancillary proteins useful for increasing glycolic acid and/or downstream product yields, titers, and/or productivities are organic acid transporter. In some embodiments, recombinant host cells comprise one or more heterologous and/or endogenous nucleic acids encoding one or more organic acid transporter proteins. Recombinant host cells comprising one or more heterologous nucleic acids encoding an organic acid transporter of the present disclosure have an increase in in glycolic acid titer and/or yield as compared to parental or control cells that do not comprise said heterologous nucleic acid(s). In some embodiments, recombinant host cells comprising one or more heterologous nucleic acids encoding an organic acid transporter produce an increased glycolic acid titer in fermentations as compared to parental or control cells that do not comprise said heterologous nucleic acid(s). In some embodiments, the glycolic acid titer is increased by 0.5 g/l, 1 g/l, 2.5 g/l, 5 g/l, 7.5 g/l, 10 g/l, or more than 10 g/l. In some embodiments, recombinant host cells comprising one or more heterologous nucleic acids encoding an organic acid transporter have increased glycolic acid yield as compared to parental or control cells that do not comprise said heterologous nucleic acid(s). In some embodiments, the glycolic acid yield is increased by 0.5%, 1%, 2.5%, 5%, 7.5%, 10%, or more than 10% (g-glycolic acid/g-substrate, or g-downstream product/g-substrate).

In many embodiments, the organic acid transporter is derived from a fungal source. In some embodiments, the organic acid transporter is selected from the group comprising Saccharomyces cerevisiae PDR12 (abbv. ScPDR12; UniProt ID: Q02785; SEQ ID NO: 35), Saccharomyces cerevisiae WAR1 (abbv. ScWAR1; UniProt ID: Q03631; SEQ ID NO: 36), Schizosaccharomyces pombe MAE1 (abbv. SpMAE1; UniProt ID: P50537: SEQ ID NO: 37), Kluyveromyces marxianus PDC12 (abbv. KmPDC12; UniProt ID: W0T9C6; SEQ ID NO: 38), Kluyveromyces lactis JEN1 (abbv. K1JEN1; UniProt 1D: Q70DJ7; SEQ ID NO: 65), Aspergillus oryzae MAE1 (abbv. AoMAE1; UniProt ID: Q2UHT6; SEQ ID NO: 67), Saccharomyces cerevisiae JEN1 (abbv. ScJEN1; UniProt ID: P36035; SEQ ID NO: 76). Kluyveromyces marxianus JEN1 (abbv. KmJEN1; UniProt ID: W0TGY6; SEQ ID NO: 77), Saccharomyces kudriavzevii JEN1-like protein (abbv. SkJEN1; UniProt ID: J6EMX9; SEQ ID NO: 78), and Candida albicans JEN1 (abbv. CaJEN1; UniProt ID: A0A1D8PKH9; SEQ ID NO: 79). In some embodiments, recombinant host cells comprise one or more heterologous nucleic acids encoding one or more proteins suitable for use in accordance with methods of the present disclosure have glycolic acid and/or downstream product transporter activity. In some embodiments, recombinant host cells comprise one or more heterologous nucleic acids encoding one or more proteins that comprise an amino acid sequence with at least 60%, at least 70%, at least 80%, at least 90%, or at least 95% sequence homology with ScPDR12 (SEQ ID NO: 35), ScWAR1 (SEQ ID NO: 36), SpMAE1 (SEQ ID NO: 37), KmPDC12 (SEQ ID NO: 38), KIJEN1 (SEQ ID NO: 63), AoMAE1 (SEQ ID NO: 67), ScJEN1 (SEQ ID NO: 76), KmJEN1 (SEQ ID NO: 77), SkJEN1 (SEQ ID NO: 78), or CaJEN1 (SEQ ID NO: 79).

### 2.4.4 Ancillary Proteins for Improved Flux Through the Glycolic Acid Pathway

Another class of ancillary proteins useful for increasing glycolic acid and/or downstream product yields, titers, and/or productivities are enzymes that increase carbon flux through the glycolic acid pathway. In the first step of the glycolic acid pathway of the present disclosure, an oxaloacetate-forming enzyme fixes carbon that originates from CO₂ onto a substrate to produce oxaloacetate. Thus. In some embodiments of the present disclosure, the ancillary proteins are proteins that aid carbon fixation. As described in preceding sections (i.e., Sections 2.2.1, 2.2.1.1, 2.2.1.2, and 2.2.1.3), the 3 oxaloacetate-forming enzymes of the present disclosure are pyruvate carboxylase, phosphoenolpyruvate carboxykinase, and phosphoenolpyruvate carboxylase. In one, some, or all of these oxaloacetate-forming enzymes, CO₂ first diffuses into the cell and is convened to bicarbonate (HCO₃⁻) and a proton by a carbon fixation enzyme. An abundant pool of bicarbonate helps the oxaloacetate-forming enzyme reaction move forward and prevents this first step of the glycolic acid pathway from becoming a bottleneck of the entire pathway.

In some embodiments, recombinant host cells comprise one or more heterologous and/or endogenous nucleic acids encoding one or more carbon fixation enzymes, in many embodiments, the carbon fixation enzyme is derived from a prokaryotic source. In many embodiments, the carbon fixation enzyme is derived from a eukaryotic source.

In some embodiments of the present disclosure, the carbon fixation enzyme is a carbonic anhydrase (EC #4.2.1.1). In some embodiments, the carbonic anhydrase is selected from the group comprising Pichia kudriavzevii carbonic anhydrase 1 (abbv. PkCAH1; UniProt ID: A0A1Z8JS57; SEQ ID NO: 44); Pichia kudriavzevii carbonic anhydrase 2 (abbv. PkCAH2; UniProt ID: A0A1V2LUA9; SEQ ID NO: 43); Homo sapiens carbonic anhydrase (abbv, HsCAH; UniProt ID: P00918; SEQ ID NO: 46). Flavia bidentis carbonic anhydrase (abbv. FbCAH; UniProt ID: P46510; SEQ ID NO: 47), Saccharomyces cerevisiae carbonic anhydrase (abbv, ScCAH; UniProt ID: P53615; SEQ ID NO: 48). Candida albicans carbonic anhydrase (abbv. CaCAH; UniProt ID: Q5AJ71; SEQ ID NO: 49), Porphyromonas gingivalis carbonic anhydrase (abbv. PgCAH; UniProt ID: Q7MV79; SEQ ID NO: 50). Mycobacterium tuberculosis carbonic anhydrase (abbv. MtCAH; UniProt ID: P9WPJ9; SEQ ID NO: 51).

In some embodiments, the carbon fixation enzyme is the Pichia kudriavzevii carbonic anhydrase 1 (abbv, PkCAH1; UniProt ID; A0A1Z8JS57; SEQ ID NO: 44). In some embodiments, the carbonic anhydrase is the Pichia kudriavzevii carbonic anhydrase 2 (abbv. PkCAH2; UniProt ID: A0A1V2LUA9; SEQ ID NO: 45). In some embodiments, the carbon fixation enzyme is the Homo sapiens carbonic anhydrase (abbv. HsCAH; UniProt ID: P00918; SEQ ID NO: 46). In some embodiments, the carbonic anhydrase is the Flaveria bidentis carbonic anhydrase (abbv. FbCAH; UniProt ID: P46510; SEQ ID NO: 47), in some embodiments, the carbonic anhydrase is the Saccharomyces cerevisae carbonic anhydrase (abbv. ScCAH; UniProt ID; P53615; SEQ ID NO: 48). In some embodiments, the carbonic anhydrase is the Candida albicans carbonic anhydrase (abbv. CaCAH; UniProt ID: Q5AJ71; SEQ ID NO: 49). In some embodiments, the carbon fixation enzyme is the Porphyromonas gingivalis carbonic anhydrase (abbv. PgCAH; UniProt ID: Q7MV79; SEQ ID NO: 50). In some embodiments, the carbon fixation enzyme is the Mycobacterium tuberculosis carbonic anhydrase (abbv. MtCAH; UniProt ID: P9WPJ9; SEQ ID NO: 51).

In some embodiments, recombinant host cells comprise one or more heterologous nucleic acids encoding one or more proteins suitable for use in accordance with methods of the present disclosure have glycolic acid and/or carbonic anhydrase activity. In some embodiments, recombinant host cells comprise one or more heterologous nucleic acids encoding one or more proteins that comprise an amino acid sequence with at least 60%, at least 70%, at least 80%, at least 90%, or at least 95% sequence homology with PkCAH1 (SEQ ID NO: 44), PkCAH2 (SEQ ID NO: 45), HsCAH (SEQ ID NO: 46). FbCAH (SEQ It NO: 47), ScCAH (SEQ ID NO: 48), CaCAH (SEQ ID NO: 49), PgCAH (SEQ ID NO: 50), or MtCAH (SEQ ID NO: 51).

Recombinant host cells comprising one or more heterologous nucleic acids encoding a carbonic anhydrase of the present disclosure have an increase in in glycolic acid titer and/or yield as compared to parental or control cells that do not comprise said heterologous nucleic acid(s). In some embodiments, recombinant host cells comprising one or more heterologous nucleic acids encoding a carbonic anhydrase produce an increased glycolic acid titer in fermentations as compared to parental or control cells that do not comprise said heterologous nucleic acid(s). In some embodiments, the glycolic acid titer is increased by 0.5 g/l, 1 g/l, 2.5 g/l, 5 g/l, 7.5 g/l, 10 g/l, or more than 10 g/l. In some embodiments, recombinant host cells comprising one or more heterologous nucleic acids encoding a carbonic anhydrase have increased glycolic acid yield as compared to parental or control cells that do not comprise said heterologous nucleic acid(s). In some embodiments, the glycolic acid yield is increased by 0.5%, 1%, 2.5%, 5%, 7.5%, 10%, or more than 10% (g-glycolic acid/g-substrate, or g-downstream product/g-substrate).

In some embodiments, recombinant host cells comprising one or more heterologous nucleic acids encoding a carbon fixation enzyme of the present disclosure further comprise increased malate production.

In some embodiments, recombinant host cells comprising one or more heterologous nucleic acids encoding a carbon fixation enzyme of the present disclosure further comprise increased oxaloacetate production.

In some embodiments, recombinant host cells comprising one or more heterologous nucleic acids encoding a carbon fixation enzyme of the present disclosure are provided fermentation conditions that comprise carbon dioxide supplementation and/or bicarbonate supplementation.

### 2.5 Decreasing or Eliminating Expression of Byproduct Pathway Enzymes

In an additional aspect, nucleic acids encoding byproduct pathway enzymes are disrupted in recombinant host cells of the present disclosure to increase glycolic acid and/or downstream product yields, productivities, and/or titers; and/or to decrease byproduct titers and/or yields as compared to control cells, or host cells that express native/undisrupted levels of said byproduct pathway enzymes. Byproduct pathway enzymes comprise any protein (excluding glycolic acid pathway enzymes and/or downstream product pathway enzymes) of any structure or function that can increase glycolic acid and/or downstream product yields, titers, and/or productivities when disrupted because they utilize intermediates or products of the glycolic acid pathway and/or the downstream product pathway.

Byproducts that accumulate during glycolic acid and/or downstream product production can lend to: (1) lower glycolic acid and/or downstream product acid titers, productivities, and/or yields; and/or (2) accumulation of byproducts in the fermentation broth that increase the difficulty of downstream purification processes. In some embodiments, recombinant host cells may comprise genetic disruptions that encompass alternations, deletions, knockouts, substitutions, promoter modifications, premature stop codons, or knock-downs that decrease byproduct accumulation. In some embodiments, recombinant host cells comprising a disruption of one or more genes encoding a byproduct pathway enzyme will have altered performance characteristics as compared to cells without said genetic disruption(s), such as decreased or eliminated byproduct pathway enzyme expression, decreased or eliminated byproduct accumulation, improved glycolic acid and/or downstream product pathway activity, altered metabolite flux through the glycolic acid and/or downstream product pathway, higher glycolic acid and/or downstream product titers, glycolic acid and/or downstream product productivities, glycolic acid and/or downstream product yields, and/or altered cellular fitness.

One important reason to decrease byproduct formation is to increase glycolic acid and/or downstream product pathway activity, resulting in an increased amount of glycolic acid and/or downstream product produced. In many embodiments, recombinant host cells of the present disclosure comprising one or more genetic disruptions of one or more genes encoding a byproduct pathway enzyme produce an increased glycolic acid and/or downstream product titer as compared to host cells that do not comprise said genetic disruption(s). In some of these embodiments, the glycolic acid titer in the fermentation broth is increased by 0.5 g/l, 1 g/l, 2.5 g/l, 5 g/l, 7.5 g/l, 10 g/l, or more than 10 g/l. In some of these embodiments, the downstream product titer in the fermentation broth is increased by 0.5 g/l, 1 g/l, 2.5 g/l, 5 g/l, 7.5 g/l, 10 g/l, or more than 10 g/l.

In addition to increasing glycolic acid and/or downstream product titers, decreasing byproduct formation can also help increase glycolic acid and/or downstream product yields. Because yield is independent of the volume of the fermentation broth, which can change during die course of a fermentation, it is often advantageous to measure glycolic acid and/or downstream product yields. In many embodiments, recombinant host cells of the present disclosure comprising one or more genetic disruptions of one or more genes encoding byproduct pathway enzymes produce an increased glycolic acid and/or downstream product yield as compared to host cells that do not comprise said genetic disruption. In some of these embodiments, the glycolic acid and/or downstream product yield is increased by 0.5%, 1%, 2.5%, 5%, 7.5%, 10%, or more than 10% (g-glycolic acid/g-substrate, or g-downstream product/g-substrate). The substrate in this yield calculation is the fermentation substrate, which is typically glucose, but may also be other, non-glucose substrates (e.g., sucrose, glycerol, or pyruvate).

Increasing glycolic acid and/or downstream product production is important for decreasing manufacturing costs, but it is also useful to disrupt genes encoding byproduct pathway enzymes in order to decrease byproduct formation. Byproducts are typically unwanted chemicals, are disposed of as waste, and their disposal can involve elaborate processing steps and containment requirements. Therefore, decreasing byproduct formation is generally also important for lowering production costs. In many embodiments, recombinant host cells of the present disclosure comprising one or more genetic disruptions of one or more genes encoding a byproduct pathway enzyme produces a lower byproduct titer as compared to host cells that do not comprise said genetic disruption. In some of these embodiments, a recombinant host cell of the disclosure comprising genetic disruption of one or more byproduct pathway enzymes produces a byproduct titer that is 0.5 g/l, 1 g/l, 2.5 g/l, 5 g/l, 7.5 g/l, 10 g/l, or greater than 10 g/l less than host cells that do not comprise said genetic disruption.

In many embodiments, recombinant host cells of the present disclosure comprising one or more genetic disruptions of one or more genes encoding a byproduct pathway enzyme produces a lower byproduct yield as compared to host cells that do not comprise said genetic disruption(s). In some of these embodiments, recombinant host cells comprise genetic disruption of one or more genes encoding byproduct pathway enzymes produce a byproduct yield that is 0.5%, 1%, 2.5%, 5%, 7.5%, 10%, or greater than 10% (g-byproduct/g-substrate) less than host cells that do not comprise said genetic disruption. As with the glycolic acid yield calculation, the substrate used in the byproduct yield calculation is the carbon source provided to the fermentation; this is typically glucose, sucrose, or glycerol, but may be any carbon substrate.

Non-limiting examples of byproducts that arise due to consumption of a glycolic acid pathway or a downstream product pathway substrate, intermediate or product include 2-phosphoglycerate, 3-phosphoglycerate, glycerol 3-phosphate, pyruvate, hydroxypyruvate, tartronate semialdehyde, 3-phosphonooxypyruvate, glyceraldehyde, I-deoxy-D-xylulose 5-phosphate, DHAP, methylglyoxal, fructose 6-phosphoric acid, inositol-3-monophosphate, and 6-phospho-glucono-1,5-lactone, acetaldehyde, carbon dioxide, acetic acid, 2-oxoglutarate, and ethanol. In the event of a redox imbalance, an undesirable excess of reduced or oxidized cofactors may also accumulate; thus, the redox cofactors NADH, NAD⁺. NADPH and NADP⁺ can also be considered byproducts.

A non-limiting list of enzyme-catalyzed reactions that utilize the glycolic acid pathway substrate (e.g., pyruvate), a glycolic acid pathway intermediate, or glycolic acid itself, is found in Table 2. Decreasing or eliminating expression of one, some or all of the genes encoding the enzymes in Table 2 can increase glycolic acid production and/or decrease byproduct production. In many cases, the product of the enzyme-catalyzed reactions provided in Table 2 can accumulate in the fermentation broth: in such cases, this indicates that expression of the native gene encoding the listed enzyme should be reduced or eliminated. For example, the occurrence of dihydroxyacetone (abbv. DHAP; also known as glycerone) in the fermentation broth indicates that expression of a native gene encoding glycerol dehydrogenase should be decreased or eliminated, in some cases, the product of the specific reaction listed in Table 2 is further converted, either spontaneously or through the action of other enzymes, into a byproduct that accumulates in the fermentation broth. For example, dihydroxyacetone is generally metabolized to glycerol, which is found to accumulate in the fermentation broth. In cases where byproduct accumulation is due to the activity of multiple enzymes, one or more of the genes encoding the one or more byproduct pathway enzymes can be deleted or disrupted to reduce byproduct formation.

In some embodiments of the present disclosure, recombinant host cells comprise microbial strains with decreased or eliminated expression of one, some or all of the genes encoding enzymes listed in Table 2. In some embodiments, recombinant host cells comprise microbial strains with decreased byproduct accumulation wherein the byproducts are formed through the activity of one, some or all of the enzymes listed in Table 2. In some embodiments, recombinant host cells comprise microbial strains with decreased expression of pyruvate-utilizing enzymes. In some embodiments, recombinant host cells comprise microbial strains with decreased expression of glycolic acid-utilizing enzymes. In some embodiments, recombinant host cells comprise microbial strains with inability to catabolize or breakdown glycolic acid and/or glycolic acid pathway intermediates. In some embodiments, recombinant host cells comprise genetic modifications that reduce the ability of the host cells to catabolize the glycolic acid and/or pathway intermediates. In some embodiments, recombinant host cells comprise genetic modifications that decrease the ability of the host cells to catabolize pyruvate except via the glycolic acid and/or downstream product pathway.

TABLE 2
Enzyme-catalyzed reactions that consume a substrate, intermediate
or product of glycolysis or the glycolic acid pathway

| Substrate | EC # | Enzyme name | Reaction formula |
|---|---|---|---|
| Pyruvate | 1.1.1.28 | Lactate | Pyruvate + Reduced cofactor + H⁺ [symbol] |
| | | dehydrogenase | Lactate + Oxidized cofactor |
| Pyruvate | 4.1.1.1 | Pyruvate | Pyruvate + H⁺ → Acetaldehyde + CO₂ |
| | | decarboxylase | |
| Pyruvate | n/a | Pyruvate | Pyruvate + CoA + Oxidized cofactor → |
| | | dehydrogenase | Acetyl-CoA + CO₂ + Reduced cofactor |
| | | complex | |
| 3- | 1.1.1.95 | Phosphoglycerate | 3-Phosphoglycerate + Oxidized cofactor |
| Phosphoglycerate | | dehydrogenase | → 3-Phosphonooxypyruvate + Reduced |
| | | | cofactor |
| Glucose 6- | 5.5.1.4 | Inositol 1- | Glucose 6-phosphate → Inositol-3- |
| phosphate | | phosphate | monophosphate |
| | | synthase | |
| Glucose 6- | 1.1.1.49 | Glucose-6- | Glucose 6-phosphate + Oxidized |
| phosphate | | phosphate | cofactor → 6-Phosphoglucono-1,5- |
| | | dehydrogenase | lactone + Reduced cofactor |
| Fructose-1,6- | 3.1.3.11 | Fructose-1,6- | Fructose-1,6-bisphosphate + H₂O → |
| biphosphate | | biphosphate | Fructose 6-phosphoric acid + |
| | | phosphatase | Orthophosphate |
| DHAP | 1.1.1.8 | Glycerol-3- | DHAP + Reduced cofactor → Oxidized |
| | | phosphate | cofactor + Glycerol 3-phosphate |
| | | dehydrogenase | |
| DHAP | 3.1.3.X | Dihydroxyacetone | DHAP + H₂O → Dihydroxyacetone + |
| | | phosphate | Phosphate |
| | | phosphatase | |
| DHAP | 4.2.3.3 | Methylglyoxal | DHAP → |
| | | synthase | Orthophosphate + Methylglyoxal |
| Glyoxylate | 2.3.3.9 | Malate synthase | Acetyl-CoA + Glyoxylate + H₂O → (S)- |
| | | | malate + CoA |
| Glyoxylate | 1.2.3.5 | Glyoxylate | Glyoxylate + H₂O + O₂ → Oxalate + |
| | | oxidase | H₂O₂ |
| Glyoxylate | 1.4.1.10 | Glycine | Glyoxylate + NH₃ + Reduced cofactor + |
| | | dehydrogenase | H⁺ → Glycine + H₂O + Oxidized |
| | | | cofactor |
| Glyoxylate | 2.6.1.X(12/4/45/35/etc) | Amino | Glyoxylate + Amino acid substrate → |
| | | transferases | Oxoacid + Amino acid product |

### 2.5.1 Decreasing or Eliminating Expression of Pyruvate Decarboxylase

Pyruvate decarboxylase (EC #14.1.1.1) catalyzes the irreversible unidirectional conversion of 1 molecule of pyruvate to 1 molecule of acetaldehyde and 1 molecule of CO₂. Pyruvate decarboxylase activity can lead to the formation of at least 3 undesirable pyruvate decarboxylase-based byproducts: acetaldehyde, acetate, and ethanol. There are at least 3 pyruvate decarboxylase homologs in P. kudriavzevii, PkPDC1 (SEQ ID NO: 39), PkPDC5 (SEQ ID NO: 40) and PkPDC6 (SEQ ID NO: 41): decreasing or eliminating expression of one or more of these homologs is useful for increasing glycolic acid production and/or decreasing accumulation of pyruvate decarboxylase-based byproducts. As described above, homologous proteins share substantial sequence homology with each other. Any protein that is homologous to one, more, or all of the pyruvate decarboxylases of the present disclosure (SEQ ID NOs. 8, 9 and 10) will share substantial sequence homology one or more of these proteins.

In some embodiments, recombinant host cells comprise genetic disruptions in one or more pyruvate decarboxylase homologs. As defined above, gene-tic disruptions encompass nucleic acid deletions, nucleic acid insertions, nucleic acid substitutions, nucleic acid mutations, premature stop codons and promoter modifications. In some embodiments, recombinant host cells of the present disclosure comprise a genetic disruption of a homologous pyruvate decarboxylase gene with at least 60%, at least 70%, at least 80%, at least 90%, at least 95%, or more than 95% homology when compared to PkPDC1, PkPDC5 or PkPDC6. In some of these embodiments, the recombinant host cell is a P. kudriavzevii strain. In some embodiments, recombinant host cells comprise one or more gene disruptions that produce altered, decreased or eliminated activity in 1, 2 or all 3, pyruvate decarboxylase proteins. In some of these other embodiments, the recombinant host cell is a P. kudriavzevii strain.

In some embodiments, recombinant host cells comprise heterologous nucleic acids encoding glycolic pathway enzymes, and further comprise one or more genetic disruptions of one, more, or all of the pyruvate decarboxylase homologs. In certain embodiments, acetaldehyde byproduct titer (i.e., g of byproduct liter of fermentation volume) at the end of fermentation is 10 g or less, preferably 5 g/l or less, and most preferably 2.5 g/l or less. In certain embodiments, acetaldehyde byproduct yield (i.e., percentage of g of byproduct/g of substrate) at the end of fermentation is 10% or less, 5% or less, 2.5% or less, and preferably, 1% or less. In certain embodiments, acetate byproduct titer at the end of fermentation is 10 g/l or less, preferably 5 g/l or less, and most preferably 25 g/l or less. In certain embodiments, acetate byproduct yield at the end of fermentation is 10% or less, 5% or less, 2.5% or less, and preferably, 1% or less. In some embodiments, ethanol byproduct titer at the end of a fermentation is 10 g/l or less, preferably 5 g/l or less, and most preferably 2.5 g/l or less. In certain embodiments, ethanol byproduct yield at the end of fermentation is 10% or less, 5% or less, 2.5% or less, and preferably, 1% or less.

Recombinant host cells comprising one or more heterologous nucleic acids encoding one or more glycolic acid pathway enzymes and further comprising one or more genetic disruptions of one or more pyruvate decarboxylase homologs have an increase in in glycolic acid titer and/or yield as compared to parental or control cells that do not comprise said genetic disruptions. In some embodiments, recombinant host cells comprising one or more heterologous nucleic acids encoding one or more glycolic acid pathway enzymes and further comprising one or more genetic disruptions of one or more pyruvate decarboxylase homologs produce an increased glycolic acid titer in fermentations as compared to parental or control cells that do not comprise said genetic disruptions. In some embodiments, the glycolic acid titer is increased by 0.5 g/l, 1 g/l, 2.5 g/l, 5 g/l, 7.5 g/l, 10 g/l, or more than 10 g/l. In some embodiments, recombinant host cells comprising one or more heterologous nucleic acids encoding one or more glycolic acid pathway enzymes and further comprising one or more genetic disruptions of one or more pyruvate decarboxylase homologs have increased glycolic acid yield as compared to parental or control cells that do not comprise said genetic disruptions. In some embodiments, the glycolic acid yield is increased by 0.5%, 1%, 2.5%, 5%, 7.5%, 10%, or more than 10% (g-glycolic acid/g-substrate, or g-downstream product/g-substrate).

### 2.5.2 Decreasing or Eliminating Expression of Pyruvate Dehydrogenase Complex

The pyruvate dehydrogenase complex catalyzes the irreversible/unidirectional conversion of 1 molecule of pyruvate, 1 molecule of coenzyme A, and 1 molecule of NAD⁺ to 1 molecule of acetyl-CoA, 1 molecule of CO₂ and 1 molecule of NADH: in wild type P. kudriavzevii, this enzyme is localized in the mitochondria: In most native microbes, pyruvate dehydrogenase is used for aerobic metabolism of pyruvate to CO₂ through the activity of the tricarboxylic acid cycle enzymes. Genetic disruption of one or more genes encoding a protein of the pyruvate dehydrogenase complex can decrease pyruvate dehydrogenase complex protein activity or expression, consequently increasing glycolic acid production and/or decreasing CO₂ byproduct formation. In some embodiments of the present disclosure, recombinant host cells comprise decreased or eliminated expression and/or activity of one or more pyruvate dehydrogenase complex proteins. In some of these embodiments, recombinant host cells comprise decreased or eliminated expression and/or activity of the E1 α-subunit of the pyruvate dehydrogenase complex (abbv. PkPDA1; SEQ ID NO: 42). In some embodiments, recombinant host cells comprise one or more heterologous nucleic acids encoding one or more proteins that comprise an amino acid sequence with at least 60%, at least 70%, at least 80%, at least 90%, or at least 95% sequence homology with SEQ ID NO: 42 In some embodiments, the recombinant host cell is a P. kudriavzevii strain.

In many embodiments wherein recombinant host cells comprise a glycolic acid pathway and genetic disruption(s) that decrease or eliminate expression and/or activity of one or more pyruvate dehydrogenase complex proteins, the glycolic acid titer and/or yield is higher as compared to recombinant host cells that do not comprise said genetic disruption(s), In some embodiments, said recombinant host cells comprise a carbon dioxide yield (i.e., g-carbon dioxide/g-glucose consumed) that is lower as compared to recombinant host cells that do not comprise said genetic disruption(s).

Recombinant host cells comprising one or more heterologous nucleic acids encoding one or more glycolic acid pathway enzymes and further comprising one or more genetic disruptions of one or more pyruvate dehydrogenase complex E1 α-subunit homologs have an increase in in glycolic acid titer and/or yield as compared to parental or control cells that do not comprise said genetic disruptions. In some embodiments, recombinant host cells comprising one or more heterologous nucleic acids encoding one or more glycolic acid pathway enzymes and further comprising one or more genetic disruptions of one or more pyruvate dehydrogenase complex E1 α-subunit homologs produce an increased glycolic acid titer in fermentations as compared to parental or control cells that do not comprise said genetic disruptions. In some embodiments, the glycolic acid titer is increased by 0.5 g/l, 1 g/l, 2.5 g/l, 5 g/l, 7.5 g/l, 10 g/l, or more than 10 g/l. In some embodiments, recombinant host cells comprising one or more heterologous nucleic acids encoding one or more glycolic acid pathway enzymes and further comprising one or more pyruvate dehydrogenase complex E1 α-subunit homologs have increased glycolic acid yield as compared to parental or control cells that do not comprise said genetic disruptions. In some embodiments, the glycolic acid yield is increased by 0.5%, 1%, 2.5%, 5%, 7.5%, 10%, or more than 10% (g-glycolic acid/g-substrate, or g-downstream product/g-substrate).

2.5.3 Decreasing or Eliminating Expression of Glycero-3-phosphate Dehydrogenase

In addition to the possible byproducts derived from 3-PG and glycolic acid pathway intermediates and product, additional byproducts can arise from intermediates in glycolysis (Table 2). Glycerol is a common byproduct that occurs under conditions of excess NADI. NAD-dependent glycerol-3-phosphate dehydrogenase (EC #1.1.1.8) catalyzes the conversion of one molecule of dihydroxyacetone phosphate (DHAP; glycerone phosphate) and one molecule of NAD(P)H to one molecule of glycerol 3-phosphate and one molecule of NAD(P)⁺, leading to the formation of the undesired byproduct glycerol. In P. kudriavzevii, NAD-dependent glycerol-3-phosphate dehydrogenase activity is the Gpd1 protein (abbv. PkGPD1; SEQ ID NO: 43).

Decreasing or eliminating the expression of PkGPD1 or its homologs is useful for decreasing glycerol byproduct accumulation. In some embodiments of the present disclosure, recombinant host cells comprise one or more genetic disruptions in one or more nucleic acids encoding a glycerol-3-phosphate dehydrogenase that gives rise to decreased, altered or eliminated expression and/or protein activity. In embodiments where the recombinant host cell is a P. kudriavzevii strain, the glycerol-3-phosphate dehydrogenase is PkGPD1.

Several amino acids in glycerol-3-phosphate dehydrogenase are highly conserved. Any protein that is homologous to PkGPD1 will comprise amino acids corresponding to a substantial number of highly conserved amino acids in PkGPD1. In some embodiments, recombinant host cells of the present disclosure comprise one or more geneic disruptions in one or more PkGPD1 homologs with at least 60%, at least 70%, at least 80%, at least 90%, at least 95%, or more than 95% homology when compared PkGPD1 (SEQ ID NO: 43).

In some embodiments, recombinant host cells comprise heterologous nucleic acids encoding glycolic acid pathway enzymes, and further comprise one or more genetic disruptions in one, more, or all PkGPD1 homologs. In certain embodiments, glycerol byproduct titer at the end of fermentation is 10 g/l or less, preferably at a titer of 5 g/l or less, and most preferably at a titer of 2.5 g/l or less. In certain embodiments, glycerol byproduct yield at the end of fermentation is 10% or less, 5% or less, 2.5% or less, and preferably, 1% or less.

Recombinant host cells comprising one or more heterologous nucleic acids encoding one or more glycolic acid pathway enzymes and further comprising one or more genetic disruptions of one or more glycerol-3-phosphate dehydrogenase homologs have an increase in in glycolic acid titer and/or yield as compared to parental or control cells that do not comprise said genetic disruptions. In some embodiments, recombinant host cells comprising one or more heterologous nucleic acids encoding one or more glycolic acid pathway enzymes and further comprising one or more genetic disruptions of one or more glycerol-3-phosphate dehydrogenase homologs produce an increased glycolic acid titer in fermentations as compared to parental or control cells that do not comprise said genetic disruptions. In some embodiments, the glycolic acid titer is increased by 0.5 g/l, 1 g/l, 2.5 g/l, 5 g/l, 7.5 g/l, 10 g/l, or more than 10 g/l. In some embodiments, recombinant host cells comprising one or more heterologous nucleic acids encoding one or more glycolic acid pathway enzymes and further comprising one or more genetic disruptions of one or more glycerol-3-phosphate dehydrogenase homologs have increased glycolic acid yield as compared to parental or control cells that do not comprise said genetic disruptions. In some embodiments, the glycolic acid yield is increased by 0.5%, %, 2.5%, 5%, 7.5%, 10% or more than I0% (g-glycolic acid/g-substrate, or g-downstream product/g-substrate).

### 2.5.4 Decreasing or Eliminating Expression of Malate Synthase

Malate synthase (EC #2.3.3.9) catalyzes the conversion of 1 molecule of glyoxylate, 1 molecule of acetyl-CoA, and 1 molecule of water to 1 molecule of malate and 1 molecule of CoA. In some embodiments, malate synthase activity can lead to acetyl-CoA and glyoxylate being siphoned away from the glycolic acid pathway, thereby decreasing glycolic acid yield. Malate accumulates as a result of malate synthase activity, which is undesirable as it forms a futile cycle within the glycolic acid pathway of the present disclosure. Decreasing or eliminating expression of one or more genes encoding a protein with malate synthase activity is useful for increasing glycolic acid production.

In some embodiments of the present disclosure, recombinant host cells comprise decreased or eliminated expression and/or activity of one or more malate synthases. In some embodiments, the recombinant host cell is a P. kudriavzevii strain.

In some embodiments, the malate synthase is the Pichia kudriavzevii malate synthase (abbv. PkMLS; UniProt ID: A0A099NZ48; SEQ ID NO: 72). In some embodiments, the malate synthase is the Saccharomyces cerevisae malate synthase 1 (abbv. ScMLS1; UniProt ID: P30952. SEQ ID NO: 731. In some embodiments, the malate synthase is the Saccharomyces cerevisiae malate synthase 2 (abbv. ScDAL7; UniProt ID: P21826; SEQ ID NO: 74).

In some embodiments, recombinant host cells comprise genetic disruptions in one or more malate synthase homologs. In some embodiments, recombinant host cells of the present disclosure comprise a genetic disruption of a homologous malate synthase gene with at least 60%, at least 70%, at least 80%, at least 90%, at least 95%, or more than 95% homology when compared to PkMLS (SEQ ID NO: 72), ScMLS1 (SEQ ID NO: 73), or ScDAL7 (SEQ ID NO: 74). In some of these embodiments, the recombinant host cell is a P. kudriavzevii strain. In some embodiments, recombinant host cells comprise one or more gene disruptions that produce altered, decreased or eliminated activity in one, more, or all, malate synthase proteins. In some of these other embodiments, the recombinant host cell is a P. kudriavzevii strain.

In some embodiments, recombinant host cells comprise heterologous nucleic acids encoding glycolic acid pathway enzymes, and further comprise one or more genetic disruptions of one, more, or all of the malate synthase homologs. In certain embodiments, malate titer (i.e., g of malate/liter of fermentation volume) at the end of fermentation is 10 Ig or less, preferably 5 g/l or less, and most preferably 2.5 g/l or less. In certain embodiments, malate yield (i.e., percentage of g of malate/g of substrate) at the end of fermentation is 10% or less, 5% or less, 2.5% or less, and preferably, 1% or less.

Recombinant host cells comprising one or more heterologous nucleic acids encoding one or more glycolic acid pathway enzymes and further comprising one or more genetic disruptions of one or more malate synthase homologs have an increase in in glycolic acid titer and/or yield as compared to parental or control cells that do not comprise said genetic disruptions. In some embodiments, recombinant host cells comprising one or more heterologous nucleic acids encoding one or more glycolic acid pathway enzymes and further comprising one or more genetic disruptions of one or more malate synthase homologs produce an increased glycolic acid titer in fermentations as compared to parental or control cells that do not comprise said genetic disruptions. In some embodiments, the glycolic acid titer is increased by 0.5 g/l, 1 g/l, 2.5 g/l, 5 g/l, 7.5 g/l, 10 g/l or more than 10 g/l. In some embodiments, recombinant host cells comprising one or more heterologous nucleic acids encoding one or more glycolic acid pathway enzymes and further comprising one or more genetic disruptions of one or more malate synthase homologs have increased glycolic acid yield as compared to parental or control cells that do not comprise said genetic disruptions. In some embodiments, the glycolic acid yield is increased by 0.5%, 1%, 2.5%, 5%, 7.5%, 10%, or more than 10% (g-glycolic acid/g-substrate, or g-downstream product/g-substrate).

### 2.5.5 Decreasing or Eliminating Expression of Glycine Dehydrogenase

Glycine dehydrogenase (EC #1.4.1.10) catalyzes the conversion of 1 molecule of glyoxylate, 1 molecule of NH₃, 1 molecule of NAD(P)H to 1 molecule of glycine, 1 molecule of H₂O and 1 molecule of NAD(P)⁺. In embodiments, glycine dehydrogenase activity can lead to glyoxylate being siphoned away from the glycolic acid pathway, thereby decreasing glycolic acid yield. Decreasing or eliminating expression of one or more genes encoding a protein with glycine dehydrogenase activity is useful for increasing glycolic acid production.

In some embodiments of the present disclosure, recombinant host cells comprise decreased or eliminated expression and/or activity of one or more glycine dehydrogenases. In some embodiments, the recombinant host cell is a P. kudriavzevii strain.

In some embodiments, the glycine dehydrogenase is the Saccharomyces cerevisiae glycine dehydrogenase (abbv. ScGCV2; UniProt ID: P49095: SEQ ID NO: 75).

In some embodiments, recombinant host cells comprise genetic disruptions in one or more glycine dehydrogenase homologs. In some embodiments, recombinant host cells of the present disclosure comprise a genetic disruption of a homologous malate synthase gene with at least 60%, at least 70%, at least 80%, at least 9W %, at least 95%, or more than 95% homology when compared to ScGCV2 (SEQ ID NO: 75). In some of these embodiments, the recombinant host cell is a P. kudriavzevii strain. In some embodiments, recombinant host cells comprise one or more gene disruptions that produce altered, decreased or eliminated activity in one, more, or all, glycine dehydrogenase proteins. In some of these other embodiments, the recombinant host cell is a P. kudriavzevii strain.

In some embodiments, recombinant host cells comprise heterologous nucleic acids encoding glycolic acid pathway enzymes, and further comprise one or more genetic disruptions of one, more, or all of the glycine dehydrogenase homologs. In certain embodiments, glycine byproduct titer (i, g of byproduct/liter of fermentation volume) at the end of fermentation is 10 g/l or less, preferably 5 g/l or less, and most preferably 2.5 g/I or less. In certain embodiments, glycine byproduct yield (i.e., percentage of g of byproduct/g of substrate) at the end of fermentation is 10% or less, % or less, 25% or less, and preferably, 1% or less.

Recombinant host cells comprising one or more heterologous nucleic acids encoding one or more glycolic acid pathway enzymes and further comprising one or more genetic disruptions of one or more glycine dehydrogenase homologs have an increase in in glycolic acid titer and/or yield as compared to parental or control cells that do not comprise said genetic disruptions. In some embodiments, recombinant host cells comprising one or more heterologous nucleic acids encoding one or more glycolic acid pathway enzymes and further comprising one or more genetic disruptions of one or more glycine dehydrogenase homologs produce an increased glycolic acid titer in fermentations as compared to parental or control cells that do not comprise said genetic disruptions. In some embodiments, the glycolic acid titer is increased by 0.5 g/l, 1 g/l, 2.5 g/l, 5 g/l, 7.5 g/l, 10 g/l, or more than 10 g/l. In some embodiments, recombinant host cells comprising one or more heterologous nucleic acids encoding one or more glycolic acid pathway enzymes and further comprising one or more genetic disruptions of one or more glycine dehydrogenase homologs have increased glycolic acid yield as compared to parental or control cells that do not comprise said genetic disruptions. In some embodiments, the glycolic acid yield is increased by 0.5%, 1%, 2.5%, 5%, 7.5%, 10%, or more than 10% (g-glycolic acid/g-substrate, or g-downstream product/g-substrate).

### 2.6 Genetic Engineering

Expression of glycolic acid pathway enzymes is achieved by transforming host cells with exogenous nucleic acids encoding glycolic acid pathway enzymes, producing recombinant host cells of the present disclosure. The same is true for expression or ancillary proteins. Any method can be used to introduce exogenous nucleic acids into a host cell to produce a recombinant host cell of the present disclosure. Many such methods are known to practitioners in the art. Some examples include electroporation, chemical transformation, and conjugation. After exogenous nucleic acids enter the host cell, nucleic acids may integrate in to the cell genome via homologous recombination.

Recombinant host cells of the present disclosure may comprise one or more exogenous nucleic acid molecules/elements, as well as single or multiple copies of a particular exogenous nucleic acid molecule/element as described herein. These molecules/elements comprise transcriptional promoters, transcriptional terminators, protein coding regions, open reading frames, regulatory sites, flanking sequences for homologous recombination, and intergenic sequences.

Exogenous nucleic acids can be maintained by recombinant host cells in various ways. In some embodiments, exogenous nucleic acids are integrated into the host cell genome, in other embodiments, exogenous nucleic acids are maintained in an episomal state that can be propagated, either stably or transiently, to daughter cells. Exogenous nucleic acids may comprise selectable markers to ensure propagation. In some embodiments, the exogenous nucleic acids are maintained in recombinant host cells with selectable markers. In some embodiments, the selectable markers are removed and exogenous nucleic acids are maintained in a recombinant host cell strain without selection. In some embodiments, removal of selectable markers is advantageous for downstream processing and purification of the fermentation product.

In some embodiments, endogenous nucleic acids (i.e., genomic or chromosomal elements of a host cell), are genetically disrupted to alter, mutate, modify, modulate, disrupt, enhance, remove, or inactivate a gene product. In some embodiments, genetic disruptions alter expression or activity of proteins native to a host cell. In some embodiments, genetic disruptions circumvent unwanted byproduct formation or byproduct accumulation. Genetic disruptions occur according to the principle of homologous recombination via methods well known in the art. Disrupted endogenous nucleic acids can comprise open reading frames as well as genetic material that is not translated into protein, in some embodiments, one or more marker genes replace deleted genes by homologous recombination. In some of these embodiments, the one or more marker genes are later removed from the chromosome using techniques known to practitioners in the art.

### SECTION 3. METHODS OF PRODUCING GLYCOLIC ACID, GLYCOLATE SALTS, AND/OR DOWNSTREAM PRODUCTS WITH RECOMBINANT HOST CELLS

Methods are provided herein for producing glycolic acid, glycolate salts, and/or one or more downstream products from recombinant host cells of the present disclosure, in certain embodiments, the methods comprise the steps of: (1) culturing recombinant host cells as provided by the present disclosure in a fermentation broth containing at least one carbon source and one nitrogen source under conditions such that glycolate is produced; and (2) recovering the glycolate, glycolic acid or glycolate salt from the fermentation broth. In some embodiments, the glycolic acid is first converted to a glycolate salt before the glycolate salt is recovered from the fermentation broth. In some embodiments, the glycolate acid or glycolate salt is first converted to a downstream product before the downstream product is recovered from the fermentation broth. In some embodiments, the glycolic acid or glycolate salt is converted to a downstream product by recombinant host cells of the present disclosure.

3.1 Fermentative Production of Glycolic Acid, Glycolate Salts, and/or Downstream Products by Recombinant Host Cells

Any of the recombinant host cells of the present disclosure can be cultured to produce and/or secrete glycolate (i.e., glycolic acid and glycolate salt). In some embodiments, the recombinant host cells of the present disclosure can be cultured to also produce one or more downstream products. As disclosed herein, the glycolate or downstream product can then be esterified and distilled to generate a purified ester.

Materials and methods for the maintenance and growth of microbes, as well as fermentation conditions, are well known to practitioners of ordinary skill in the art. It is understood that consideration must be given to appropriate culture medium, pH, temperature, revival of frozen stocks, growth of seed cultures and seed trains, and requirements for aerobic, microaerobic, or anaerobic conditions, depending on the specific requirements of the host cells, the fermentation, and process flows.

The methods of producing glycolate and/or one or more downstream products provided herein may be performed in a suitable fermentation broth in a suitable bioreactor such as a fermentation vessel, including but not limited to a culture plate, a flask, or a fermenter. Further, the methods can be performed at any scale of fermentation known in the art to support microbial production of small-molecules on an industrial scale, Any suitable fermenter may be used including a stirred tank fermenter, an airlift fermenter, a bubble column fermenter, a fixed bed bioreactor, or any combination thereof.

In some embodiments of the present disclosure, the fermentation broth is any fermentation broth in which a recombinant host cell capable of producing glycolate according to the present disclosure, and can subsist (i.e., maintain growth, viability, and/or catabolize glucose or other carbon source). In some embodiments, the fermentation broth is an aqueous medium comprising assimilable carbon, nitrogen, and phosphate sources. Such a medium can also include appropriate salts, minerals, metals, and other nutrients. In some embodiments, the carbon source and each of the essential cell nutrients are provided to the fermentation broth incrementally or continuously, and each essential cell nutrient is maintained at essentially the minimum level required for efficient assimilation by growing cells. Exemplary cell growth procedures include batch fermentation, fed-batch fermentation with batch separation, fed-batch fermentation with continuous separation, and continuous fermentation with continuous separation. These procedures are well known to practitioners of ordinary skill in the art.

In some embodiments of the present disclosure, the handling and culturing of recombinant host cells to produce glycolate and/or downstream product may be divided up into phases, such as growth phase, production phase, and/or recovery phase. The following paragraphs provide examples of features or purposes that may relate to these different phases. One skilled in the art will recognize that these features or purposes may vary based on the recombinant host cells used, the desired glycolate and/or downstream product yield, titer, and/or productivity, or other factors. While it may be beneficial in some embodiments for the glycolic acid pathway enzymes, ancillary proteins and/or endogenous host cell proteins to be constitutively expressed, in other embodiments, it may be preferable to selectively express or repress any or all of the aforementioned proteins.

During growth phase, recombinant host cells may be cultured to focus on growing cell biomass by utilizing the carbon source provided. In some embodiments, expression of glycolic acid pathway enzymes and/or ancillary proteins are repressed or uninduced. In some embodiments, no appreciable amount of glycolate, downstream product, or any of their pathway intermediates are made. In some embodiments, proteins that contribute to cell growth and/or cellular processes may be selectively expressed.

During production phase, however, recombinant host cells may be cultured to stop producing cell biomass and to focus on glycolate and/or downstream product biosynthesis by utilizing the carbon source provided. In some embodiments, glycolic acid pathway enzymes, downstream product pathway enzymes, and/or ancillary proteins may be selectively expressed during production to generate high product titers, yields and productivities. The production phase is synonymous with fermentation, fermentation run or fermentation phase.

In some embodiments, the growth and production phases take place at the same time. In other embodiments, the growth and production phases are separate. While in some embodiments, product is made exclusively during production phase, in other embodiments some product is made during growth phase before production phase begins.

The recovery phase marks the end of the production phase, during which cellular biomass is separated from fermentation broth and glycolate or downstream product is purified from fermentation broth. Those skilled in the art will recognize that in some fermentation process, e.g., fill-draw and continuous fermentations, there may be multiple recovery phases where fermentation broth containing biomass and glycolic acid are removed from the fermentation system. The draws of fermentation broth may be processed independently or may be stored, pooled, and processed together. In other fermentation processes, e.g., batch and fed-batch fermentations, there may only be a single recovery phase.

Fermentation procedures are particularly useful for the biosynthetic production of commercial glycolate and/or downstream product. It is understood by practitioners of ordinary skill in the art that fermentation procedures can be scaled up for manufacturing glycolate and/or downstream product and exemplary fermentation procedures include, for example, fed-batch fermentation and batch product separation: fed-batch fermentation and continuous product separation: batch fermentation and batch product separation; and continuous fermentation and continuous product separation.

### 3.1.1 Carbon Source

The carbon source provided to the fermentation can be any carbon source that can be fermented by recombinant host cells. Suitable carbon sources include, but are not limited to, monosaccharides, disaccharides, polysaccharides, glycerol, acetate, ethanol, methanol, methane, or one or more combinations thereof, Exemplary monosaccharides suitable for use in accordance to the methods of the present disclosure include, but are not limited to, dextrose (glucose), fructose, galactose, xylose, arabinose, and any combination thereof. Exemplary disaccharides suitable for use in accordance to the methods of the present disclosure include, but are not limited to, sucrose, lactose, maltose, trehalose, cellobiose, and any combination thereof. Exemplary polysaccharides suitable for use in accordance to the methods of the present disclosure include, but are not limited to, starch, glycogen, cellulose, and combinations thereof. In some embodiments, the carbon source is dextrose. In other embodiments, the carbon source is sucrose. In some embodiments, mixtures of some or all the aforementioned carbon sources can be used in fermentation.

In some embodiments, an additional carbon source is provided to the fermentation in the form of gaseous carbon dioxide. In certain embodiments, the glycolic acid pathway of the present disclosure comprises a phosphoenolpyruvate carboxykinase (PCK) which converts phosphoenolpyruvate to oxaloacetate with the incorporation of carbon dioxide. Carbon dioxide supplementation during fermentation may improve PCK catalytic efficiency, thereby increasing overall glycolic acid pathway flux. In some embodiments, carbon dioxide supplementation increases glycolic acid yields, titers, and/or productivities. In various embodiments, fermentation conditions comprise at least 2.5%, at least 5%, at least 10%, or at least 15% carbon dioxide in air supplementation. In some embodiments, carbon dioxide supplementation is provided during production phase (i.e., not during biomass/growth phase).

In some embodiments, an additional carbon source is provided to the fermentation in the form of bicarbonate. In certain embodiments, the glycolic acid pathway of the present disclosure comprises a phosphoenolpyruvate carboxylase (PPC) which converts phosphoenolpyruvate to oxaloacetate with the incorporation of bicarbonate. Further, in the cytosol, carbon dioxide is converted to bicarbonate either by the enzyme carbonic anhydrase or spontaneously via carbonic acid. Carbon dioxide supplementation and/or bicarbonate supplementation during fermentation may improve PPC catalytic efficiency, thereby increasing overall glycolic acid pathway flux. In some embodiments, carbon dioxide supplementation and/or bicarbonate supplementation increases glycolic acid yields, titers, and/or productivities. In various embodiments, the fermenter is sparged with air comprising at least 2.5%, at least 5%, at least 10%, or at least 15% carbon dioxide as a percentage of total gases. In various embodiments, the fermentation medium contains at least 5 mM, at least 10 mM, at least 15 mM, or at least 30 mM bicarbonate. In some embodiments, fermentation conditions comprise supplementing up to 15 mM sodium bicarbonate (NaHCO₃), or up to 30 mM (NaHCO₃) in the fermentation broth. In some embodiments, carbon dioxide supplementation and/or bicarbonate supplementation is provided during production phase (i.e., not during biomass/growth phase).

3.1.2 pH

The pH of the fermentation broth can be controlled by the addition of acid or base to the culture medium, Preferably, fermentation pH is controlled at the beginning of fermentation and then allowed to drop as glycolic acid accumulates in the broth, minimizing the amount of base added to the fermentation (thereby improving process economics) as well as minimizing the amount of salt formed. Specifically, the pH during fermentation is maintained in the range of 2-8, and more preferably, in the range of4-8. At the end of fermentation, the final pH is in the range of 2-5. Non-limiting examples of suitable acids used to control fermentation pH include aspartic acid, acetic acid, hydrochloric acid, and sulfuric acid. Non-limiting examples ofsuitable bases used to control fermentation pH include sodium bicarbonate (NaHCO₃), sodium hydroxide (NaOH), potassium bicarbonate (KHCO3), potassium hydroxide (KOH), calcium hydroxide (Ca(OH)₂), calcium carbonate (CaCO₃), ammonia, ammonium hydroxide, and diammonium phosphate. In some embodiments, a concentrated acid or concentrated base is used to limit dilution of the fermentation broth.

Base cations and glycolate unions react to form ionic compounds in fermentation broths. Base cations and downstream product anions also react to form ionic compounds in fermentation broths. For example, base Na+ cations and glycolate anions react to form sodium glycolate. In some embodiments, the ionic compounds formed by base cations and glycolate anions are soluble in fermentation broth. In other embodiments, the ionic compounds formed by base cations and glycolate anions are insoluble salts and may crystallize in the fermentation broth,

### 3.1.3 Temperature

The temperature of the fermentation broth can be any temperature suitable for growth of the recombinant host cells and/or production of glycolic acid. Preferably, during glycolic acid production, the fermentation broth is maintained within a temperature range of from about 20° C. to about 45° C., and more preferably in the range of from about 25° C. to about 42° C.

### 3.1.4 Oxygen/Aeration

Under aerobic conditions, microbes will commonly use molecular oxygen as an electron acceptor to reoxidize redox cofactors. If the fermentation is not appropriately oxygenated, glycolate and/or downstream product production will decrease. During cultivation, aeration and agitation conditions are selected to produce an oxygen transfer rate (OTR: rate of dissolution of dissolved oxygen in a fermentation medium) that results in glycolic acid formation. In various embodiments, fermentation conditions are selected to produce an OTR of greater than 10 mmol/l/hr. In some embodiment, fermentation conditions are selected to produce an OTR of greater than 20 mmol/l/hr, greater than 30 mmol/l/hr, greater than 40 mmol/l/hr, greater than 50 mmol/l/hr, greater than 75 mmol/l/hr, greater than 100 mmol/l/hr, greater than 125 mmol/l/hr, greater than 150 mmol/l/hr, greater than 175 mmol/l/hr, or greater than 200 mmol/l/hr. OTR as used herein refers to the volumetric rate at which oxygen is consumed during the fermentation. Inlet and outlet oxygen concentrations can be measured by exhaust gas analysis, for example by mass spectrometers. OTR can be calculated by one of ordinary skill in the art using the Direct Method described in Bioreaction Engineering Principles 3^{rd} Edition, 2011, Spring Science+Business Media, p. 449. The recombinant host cells of the present disclosure are able to produce glycolic acid and/or downstream product under a wide range of oxygen concentrations.

### 3.1.5 Yields and Titers

A high yield of glycolic acid, glycolate, and/or downstream product from the provided carbon source(s) is desirable to decrease the production cost. As used herein, yield is calculated as the percentage of the mass of carbon source catabolized by recombinant host cells of the present disclosure and used to produce glycolic acid, glycolate, and/or downstream product. In some cases, only a fraction of the carbon source provided to a fermentation is catabolized by the cells, and the remainder is found unconsumed in the fermentation broth or is consumed by contaminating microbes in the fermentation. Thus, in various aspects the fermentation is both substantially pure of contaminating microbes and the concentration of unconsumed carbon source at the completion of the fermentation is measured. For example, if 100 grams of glucose is fed into the fermentation, and at the end of the fermentation 25 grams of glycolic acid are produced and there remains 10 grams of glucose, the glycolic acid yield is 27.7% (i.e., percentage of 25 grams glycolic acid from 90 grams glucose). In certain embodiments of the methods provided herein, the final yield of glycolic acid on the carbon source is at least 10%, at least 20%, at least 30% at least 35%, at least 40% at least 50%, at least 60%, at least 70%, at least 80%, or greater than 80%. In certain embodiments, the recombinant host cells provided herein are capable of producing at least 70%, at least 75%, or greater than 80% by weight of carbon source to glycolic acid. As described above under Definitions, glycolic acid can also exist as glycolate anion depending on the pH, and that the glycolate anion can form a salt. Those skilled in the art will recognize that when a glycolate salt is found in the fermentation broth, the glycolic acid yield can be determined by calculating the mols of glycolate salt present and adjusting for the molecular weight difference between the glycolate salt and glycolic acid.

In addition to yield, the titer (or concentration) of glycolic acid, glycolate, and/or downstream product in the fermentation is another important metric for production. Assuming all other metrics are equal, a higher titer is preferred to a lower titer. Generally speaking, titer is provided as grams of product (e.g., glycolic acid, glycolate, and/or downstream product) per liter of fermentation broth (i.e., g/l). In some embodiments, glycolic acid, glycolate, and/or downstream product titer is at least 1 g/l, at least 5 g/l, at least 10 g/l, at least 15 g/l, at least 20 g/l, at least 25 g/l, at least 30 g/l, at least 40 g/l, at least 50 g/l, at least 60 g/l, at least 70 g/l, at least 80 g/l, at least 90 g/l, at least 100 g/l, or greater than 100 g/l at some point during the fermentation, and preferably at the conclusion of the fermentation. As with yield calculations, those skilled in the art will recognize glycolic acid can also exist as a glycolate salt, and that a glycolic acid titer can be calculated from the glycolate salt titer by adjusting for molecular weight differences between the glycolate salt and glycolic acid.

Further, productivity, or the rate of product (i.e., glycolic acid, glycolate, or downstream product) formation, is important for decreasing production cost, and, assuming all other metrics are equal a higher productivity is preferred over a lower productivity. Generally speaking, productivity is provided as grams product produced per liter of fermentation broth per hour (i.e., g/l/hr). In some embodiments, glycolic acid, glycolate, and/or downstream product productivity is at least 0.1 g/l/hr, at least 0.25 g/l/hr, at least 0.5 g/l/hr, at least 0.75 g/l/hr, at least 1.0 g/l/hr, at least 125 g/l/hr, at least 1.5 g/l/hr, or greater than 1.5 g/l/hr over some time period during the fermentation. As with yield and titer calculations, those skilled in the art will recognize glycolic acid can also exist as a glycolate salt, and that a glycolic acid productivity can be calculated from the glycolate salt productivity by adjusting for molecular weight differences between the glycolate salt and glycolic acid.

Practitioners of ordinary skill in the art understand that HPLC is an appropriate method to determine the amount of glycolate and/or downstream product produced, the amount of any byproducts produced (e.g., organic acids and alcohols), the amount of any pathway metabolite or intermediate produced, and the amount of unconsumed glucose left in the fermentation broth. Aliquots of fermentation broth can be isolated for analysis at any time during fermentation, as well as at the end of fermentation. Briefly, molecules in the fermentation broth are first separated by liquid chromatography (LC); then, specific liquid fractions are selected for analysis using an appropriate method of detection (e.g., UV-VIS, refractive index, and/or photodiode array detectors). In some embodiments of the present disclosure, a salt (e.g., glycolate and/or downstream product) is the fermentative product present in the fermentation broth. Practitioners in the art understand that the salt is acidified before or during HPLC analysis to produce glycolic acid or corresponding organic acid of a downstream product. Hence, the acid concentration calculated by HPLC analysis can be used to calculate the salt titer in the fermentation broth by adjusting for difference in molecular weight between the 2 compounds.

Gas chromatography-mass spectrometry (GC-MS) is also an appropriate method to determine the amount of target product and byproducts, particularly if they are volatile. Samples of fermentation can be isolated any time during and after fermentation and volatile compounds in the headspace can be extracted for analysis. Non-volatile compounds in the fermentation medium (e.g., organic acids) can also be analyzed by (C-MS after derivatization (i.e., chemical alteration) for detection by GC-MS. Non-volatile compounds can also be extracted from fermentation medium by sufficiently increasing the temperature of the fermentation medium, causing non-volatile compounds to transition into gas phase for detection by GC-MS. Practitioners in the art understand that molecules are carried by an inert gas carries as they move through a column for separation and then arrive at a detector.

### SECTION 4. EXAMPLES

### Parent Strain Used in the Examples

The parent strain in Example 1 was a P. kudriavzevii strain auxotrophic for histidine and uracil due to genetic disruptions in UR-A2 and HIS3 (i.e., the strain cannot grow in media without histidine and uracil supplementation). Histidine auxotrophy in the parent strain enables selection of new, engineered strains that carty a HIS3 marker, enabling histidine prototrophy and indicating desired nucleic acid modification. Likewise, uracil auxotrophy in the parent strain enables selection of new, engineered strains that carry a URA2 maker, enabling uracil prototrophy and indicating desired nucleic acid modification. Thus, cells that were successfully modified with exogenous nucleic acids to comprise desired genetic modifications can grow in media without histidine and/or uracil supplementation, dependent on the selection marker included in the exogenous nucleic acid. Following confirmation of correct strain engineering, the selection marker(s) were removed by, for example, homologous recombination and marker loopout. Removing the marker enables subsequent rounds of strain engineering using the same selection markers.

### Media Used in the Examples

Complete supplement mixture (CSM) medium. CSM medium comprised Adenine 10 mg/L; L-Arginine HCl 50 mg/L; L-Aspartic Acid 80 mg/L; L-Histidine HCl 20 mg/L; L-Isoleucine 50 mg/L; L-Leucine 100 mg/L; L-Lysine HCl 50 mg/L; L-Methionine 20 mg/L; L-Phenylalanine 50 mg/L; L-Threonine 100 mg/L; L-Tryptophan 50 mg/L; L-Tyrosine 50 mg/L; Uracil 20 mg/L; L-Valine 140 mg/L. The YNB used in the CSM comprised Ammonium sulfate 5.0 g/L, Biotin 2.0 μg/L, Calcium pantothenate 400 μg/L Folic acid 2.0 μg/L. Inositol 2.0 mg/L Nicotinic acid 0-400 μg/L p-Aminobenzoic acid 200 μg/L, Pyridoxine HCl 400 μg/L, Riboflavin 200 μg/L, Thiamine HCl 400 μg/L, Boric acid 500 μg/L, Copper sulfate 40 μg/L, Potassium iodide 100 μg/L, Ferric chloride 200 μg/L, Manganese sulfate 400 μg/L, Sodium molybdate 200 μg/L, Zinc sulfate 400 μg/L, Potassium phosphate monobasic 1.0 g/L, Magnesium sulfate 0.5 g/L, Sodium chloride 0.1 g/L, and Calcium chloride 0.1 g/L.

Complete supplement mixture minus histidine (CSM-His) medium. CSM-His medium is identical to CSM medium with the exception that histidine was not included in the medium. Engineered strains auxotrophic for histidine are unable to grow on CSM-His medium while engineered strains containing exogenous nucleic acids comprising a histidine selectable marker (e.g., HIS3) are capable of growth in CSM-His medium.

Complete supplement mixture minus uracil (CSM-Ura) medium. CSM-Ura medium is identical to CSM medium with the exception that uracil was not included in the medium, Engineered strains auxotrophic for uracil are unable to grow on CSM-Ura medium while engineered strains containing exogenous nucleic acids comprising a uracil selectable marker (e.g., URA2) are capable of growth in CSM-Ura medium.

BM02 medium. BM02 medium is Glucose 125 g/l, K₂SO₄ 0.816 g/l, Na₂SO₄ 0.1236, MgSO₃-7H₂O 0.304 g/l, Urea 4.3 g/l, Myo-inositol 2 mg/l, Thiamin HCl 0.4 mg/l, Pyridoxal HCl 0.4 mg/l, Niacin 0.4 mg/l, Ca-Pantothenate 0.4 mg/l, Biotin μg/l, Folic acid 2 μg/l. PABA 200 μg/l, Riboflavin 200 μg/l, Boric acid 0.25 mg/l. Copper sulfate pentahydrate 393 μg/l, Iron sulfate 11.0 mg/l, Manganese chloride 1.6 mg/l, Sodium molybdate 100 μg/l, Zinc sulfate 4 mg/l, and EDTA 11 mg/l.

BM02-P medium. BM02-P medium is BM02 medium with 1 g/l potassium phosphate.

YPE medium. YPE medium is Bacto peptone 20 g/L Yeast extract 10 g/l, and Ethanol 2% (v/v)

### Example 1: Construction of Recombinant P. kudriavzevii Strain, LPK15779, with Eliminated Expression of Pyruvate Decarboxylase

Example 1 describes the construction of a pyruvate decarboxylase (PDC) minus P. kudriavzevii, LPK15779, wherein all 3 PDC genes, i.e., Pdc1, Pdc5 and Pdc6, were genetically disrupted to eliminate expression of PkPDC1 (SEQ ID NO: 39), PkPDC5 (SEQ ID NO: 40), and PkPDC6 (SEQ ID NO: 41).

The parent P. kudriavzevii strain used in this example was auxotrophic for uracil and histidine. To eliminate PDC expression, the Pdc1, Pdc5 and Pdc6 genes in the P. kudriavzevii genome were disrupted sequentially. The P. kudriavzevii strain was diploid and 2 copies of each pyruvate decarboxylase gene were present at the indicated locus; therefore, disruption of each gene was achieved by deleting of both gene copies.

A URA3 selectable marker, amplified by PCR, was provided to the parent P. kudriavzevii strain to complement the uracil auxotrophic deficiency. The URA3 selectable marker comprised unique upstream and downstream homologous regions for homologous recombination at the P. kudriavzevii Pdc1 locus, a transcriptional promoter, a URA3 coding region, and a transcriptional terminator. The transcriptional promoter 5′ of URA3 was the P. kudriavzevii TEF promoter (pPkTEF1) and the transcriptional terminator 3′ of URA3 was the S. cerevisiae TDH3 terminator (tScTDH3). The PCR product of the URA3 selectable marker was gel-purified and provided as exogenous nucleic acids to P. kudriavzevii. Transformation was carried out in a single step and gene deletion was achieved by homologous recombination. Transformants were selected on CSM-Ura medium and successful deletion of both copies of the gene encoding PkPDC1 was confirmed by genetic sequencing of this locus and the flanking regions. After successful construction of a recombinant P. kudriavzevii comprising a Pdc1 genetic disruption, the URA3 selectable marker was removed from the recombinant strain genome by recombination and marker loopout.

The URA3 selectable marker and genetic disruption strategy described above were reused to next disrupt the Pdc5 and Pdc6 genes in succession. Deletion of the native genes encoding PkPDC5 and PkPDC6 was confirmed by genetic sequencing of this locus and the flanking regions. The P. kudriavzevii strain that resulted from Example 1, LPK15779, was without any URA3 selectable marker. The URA3 selectable marker was absent in the following examples that describe further strain engineering or strain performance testing. Thus, Example 1 produced a PDC minus (i.e., comprises deletion of native genes encoding PkPDC1, PkPDC5, and PkPDC6), uracil and histidine auxotrophic P. kudriavzevii, which was the background strain for Example 2 below.

### Example 2: Construction of Recombinant P. kudriavzevii Background Strain, LPK15942, with Eliminated Expression of Pyruvate Decarboxylase and Pyruvate Dehydrogenase Complex

Example 2 describes the construction of a pyruvate dehydrogenase complex (PDH) minus P. kudriavzevii, LPK15942, wherein expression of PDH was eliminated via genetic disruption of the Pda1 gene. Pda1 encodes for the E1 α-subunit (PkPDA1. SEQ ID NO: 42) of the PDH. When PkPDA1 expression is eliminated, PDH cannot assemble into a functional complex. Thus, PDH expression is also eliminated and the recombinant host cell is unable to catalyze the conversion of pyruvate, coenzyme A and NAD⁺ to acetyl-CoA, CO₂ and NADH in the host cell mitochondria. This genetic disruption has the end result of decreasing respiration, thereby decreasing formation of byproduct CO₂ and increasing glycolic acid production.

PkPDA1 was genetically disrupted using the same engineering strategy as described above in Example 1. LPK15779, a PDC minus, uracil and histidine auxotrophic P. kudriavzevii strain from Example 1 was the background strain used in Example 2.

A HIS3 selectable marker, amplified by PCR, was provided to the background strain (from Example 1) to complement the histidine auxotrophic deficiency. The HIS3 selectable marker comprised unique upstream and downstream homologous regions for homologous recombination at the PdaI locus of the background strain genome, a transcriptional promoter, a HIS3 coding region, and a transcriptional terminator. The transcriptional promoter 5′ of H1S3 was the P. kudriavzevii TEF1 promoter (pPkTEF1) and the transcriptional terminator 3 of HIS3 was the S. cerevisiae TDH3 terminator (tScTDH3). The PCR product of the HIS3 selectable marker was gel-purified and provided as exogenous nucleic acids to the background strain. Transformation was carried out in a single step and gene deletion was achieved by homologous recombination. Transformants were selected on CSM-His medium and successful deletion of both copies of the genes encoding PkPDA1 was confirmed by genetic sequencing of this locus and the flanking regions. After successful construction of a recombinant P. kudriavzevii comprising a Pda1 genetic disruption, the HIS3 selectable marker was removed from the recombinant strain genome by recombination and marker loopout.

The P. kudriavzevii strain that resulted from Example 2, LPK15942, was without a HIS3 selectable marker. The HIS3 selectable marker was absent in the following examples that describe further strain engineering or strain performance testing. Example 2 produced a PDC minus, PDH minus, uracil and histidine auxotrophic P. kudriavzevii (i.e., the strain comprised deletion of native genes encoding PkPDC1, PkPDC5, PkPDC6, and PkPDA1), which was the background strain used in Example 3.

### Example 3: Recombinant P. kudriavzevii Background Strain, LPK15942, Did not Naturally Produce Glycolic Acid

Example 3 describes the culturing and analysis of LPK15942 (from Example 2) for glycolic acid production before LPK15942 was used as the background strain for genomic integration of the glycolic acid pathway (Example 4 below). LPK15942 colonies were used to inoculate replicate tubes of 15 ml of YPE medium and were incubated at 30° C. with 80% humidity and shaking at 200 rpm for 20 hours. These replicate tubes of pre-cultures were used to inoculate baffled flask replicates of 250 ml of BM02-P media with 12.5% glucose, 1% ethanol and 40 g/l CaCO₃. Pre-cultures were diluted with water for OD₆₀₀ measurements to inform appropriate dilution of pre-cultures to produce a starting culture biomass of 1 g/l dry cell weight (DCW). Baffled flask cultures were then incubated at 30° C. with 80% humidity and shaking at 200 rpm. After 48 hours, the cultures were diluted 3× with 1 M HCl, spin-filtered and frozen for storage. Samples were analyzed by HPLC within 48 hours of harvest.

For HPLC analysis, frozen samples were thawed and analyzed by HPLC using a Bio-Rad Aminex 87H column (300×7.8 mm) and a Bio-Rad Fermentation Monitoring column (#1250115; 150×7.8 mm) installed in series, with an isocratic elution rate of 0.8 ml/min with water at pH 1.95 (with sulfuric acid) at 30° C. Refractive index and UV 210 nm measurements were acquired for 35 minutes.

The LPK15942 background strain did not produce detectable amounts of glycolic acid. Thus, all engineered P. kudriavzevii strains built from this background strain were incapable of producing glycolic acid without the heterologous nucleic acids that encode the glycolic acid pathway.

### Example 4: Construction of Recombinant P. kudriavzevii Strains LPK152375 and LPK153341 Comprising ScPYC2, ScMDH3, and SpMAE1, and Genetic Disruption of GPD1

Example 4 describes the construction of recombinant P. kudriavzevii host cells of the present disclosure that each comprised heterologous nucleic acids encoding the first 2 enzymes of the glycolic acid pathway: ScPYC2 (SEQ ID NO: 1) and ScMDH3 (SEQ ID NO: 4); the organic acid transporter SpMAE1 (SEQ ID NO: 37); and further comprised genetic disruption of both copies of PkGPD1 (SEQ ID NO: 43) (i.e. producing a GPD minus phenotype).

The PDC minus, PDH minus, uracil and histidine auxotrophic P. kudriavzevii, LPK15942 from Example 2 was the background strain used in this example to construct LPK152375 and LPK153341.

The heterologous nucleic acids used in this example were codon-optimized for yeast and were synthesized and provided by Twist Bioscience; each gene was cloned into its own entry vector, pEV, along with an upstream transcriptional promoter and a downstream transcriptional terminator. The transcriptional promoters cloned in front (5′) of each gene were constitutive and derived from P. kudriavzevii. The transcriptional terminators cloned behind (3′) of each gene were derived from S. cerevisiae. The promoter and terminator for ScMDH3 was the P. kudriavzevii TDH1 promoter (pPkTDH1) and the S. cerevisiae GRE3 terminator (tScGRE3), respectively. The promoter and terminator for SpMAE1 was the P. kudriavzevii PGK) promoter (pPkPGK1) and the S. cerevisiae TPI1 terminator (tScTPI1), respectively. The promoter and terminator for ScPYC2 was the P. kudriavzevii ENO1 promoter (pPkENO1) and the S. cerevisiae PYC2 terminator (tScPYC2), respectively. Additionally, a HIS3 marker was included in the heterologous expression cassette to complement the histidine auxotrophic deficiency in the parent strain. This HIS3 marker comprised a transcriptional promoter, a HIS3 coding region, and a transcriptional terminator. The transcriptional promoter 5′ of HIS3 was the P. kudriavzevii TEF1 promoter (pPkTEF1) and the transcriptional terminator 3′ of HIS3 was the S. cerevisiae TDH3 terminator (tScTDH3).

Each gene was amplified from its respective pEV vector using primers with upstream and downstream homologous regions to neighboring genetic elements to drive correct assembly of the full-length pathway. The upstream and downstream homologous regions were 25 bp to 700 bp in length. The 5′ and 3′ ends of the expression cassette comprised regions homologous to the genomic sequences upstream and downstream of the P. kudriavzevii GPD1 locus, thereby facilitating integration of the heterologous nucleic acids encoding the glycolic acid pathway enzymes at the GPD1 locus in the P. kudriavzevii genome. Consequently, one or both copies of the PkGPD1 gene were deleted from the host genome; thus, genomic integration of the glycolic acid pathway simultaneously decreased or eliminated expression of PkGPD1 (SEQ ID NO: 43).

All PCR products were purified and provided as exogenous nucleic acids to P. kudriavzevii. Transformation was carried out in a single step. Transformants were selected on CSM-His medium. Successful integration of all heterologous nucleic acids encoding the first 2 glycolic acid pathway enzymes as well as deletion of both copies of the genes encoding PkGPD1 were confirmed by genetic sequencing of this locus and the flanking regions.

LPK153341 further comprised an additional 1 copy to an additional 2 copies of ScMDH3 in the P. kudriavzevii NDE1 locus. As described above, the promoter and terminator for ScMDH3 was the P. kudriavzevii TDH1 promoter (pPkTDH1) and the S. cerevisae GRE3 terminator (tScGRE3), respectively. ScMDH3 was again amplified from its respective pEV vector using primers with upstream and downstream homologous regions to neighboring genetic elements to drive correct integration into the P. kudriavzevii NDE1 locus; the upstream and downstream homologous regions were 25 bp to 700 bp in length. Consequently, one or both copies of the PkNDE1 gene were deleted from the host genome; thus, genomic integration of the additional 1 copy or additional 2 copies of ScMDH3 simultaneously decreased or eliminated expression of PkNDE1.

Example 4 Produced Recombinant Host Cells LPK152375 and LPK153341 that comprised heterologous nucleic acids encoding enzymes for the first 2 steps in the glycolic acid pathway, and the transporter SpMAE1, and further comprised genetic disruption of PkPDC1 PkPDC5, PkPDC6, PkPDA1, and PkGPD1, LPK153341 further comprised additional 1 to 2 copies of ScMDH3 and genetic disruption of PkNDE1. Both strains were additionally auxotrophic for uracil.

### Example 5: Recombinant P. kudriavzevii Strains LPK152375 and LPK153341 Produce Malate

Example 5 describes the culturing and analysis of LPK152375 and LPK153341 (from Example 4) for malate production before integration of the complete glycolic acid pathway into the recombinant host cell genome. Malate is an intermediate of the glycolic acid pathway of the present disclosure (FIG. 1).

LPK152375 and LPK153341 colonies were used to inoculate replicate tubes of 15 ml of YPE medium and were incubated at 30° C. with 80% humidity and shaking at 250 rpm for 20 hours. These replicate tubes of pre-cultures were used to inoculate baffled flask replicates of 15 ml of BM02 media with 125 g/l glucose, +/−1% ethanol, +/− 50 μg/ml uracil, +/−1.5% glutamic acid, 40 g/l CaCO₃ and 10%-15% CO₂. Pre-cultures were diluted with water for OD₆₀₀, measurements to inform appropriate dilution of pre-cultures to produce a starting culture biomass of 1 g/l dry cell weight (DCW. Baffled flask cultures were then incubated at 30° C. with 80% humidity and shaking at 200 rpm. After 48 hours, the cultures were diluted 3× with 1 M HCl, spin-filtered and frozen for storage. Samples were analyzed by HPLC within 48 hours of harvest.

For HPLC analysis, frozen samples were thawed and analyzed by HPLC using a Bio-Rad Aminex 87H column (300×7.8 mm) and a Bio-Rad Fermentation Monitoring column (#1250115; 150×7.8 mm) installed in series, with an isocratic elution rate of 0.8 ml/min with water at pH 1.95 (with sulfuric acid) at 30° C. Refractive index and UV 210 nm measurements were acquired for 35 minutes.

The LPK152375 and LPK153341 strains produced ˜6 g/l malate, i.e. at amounts higher than the background strain LPK15942 (Example 2). Thus, all engineered P. kudriavzevii strains built from LPK152375 and LPK153341 are capable of producing the glycolic acid pathway intermediate malate due to heterologous nucleic acids encoding ScPYC2, ScMDH3, and SpMAE1.

### Example 7:Construction of Recombinant P. kudriavzevii Strain LPK154945 Comprising Glycolic Acid Pathway

Example 7 describes the construction of recombinant P. kudriavzevii host cells of the present disclosure that comprised heterologous nucleic acids encoding the last 3 enzymes of the glycolic acid pathway: MeMCSA and MeMCSB (SEQ ID NO: 15 and SEQ ID NO: 16), RcMCL1 (SEQ ID NO: 24), and AtGLYR (SEQ ID NO: 19).

LPK152375 from Example 4 (with heterologous nucleic acids expressing ScPYC2 and ScMDH3) was the background strain used in this example to construct LPK154945.

The heterologous nucleic acids used in this example were codon-optimized for yeast and were synthesized and provided by Twist Bioscience; each gene was cloned into its own entry vector, pEV, along with an upstream transcriptional promoter and a downstream transcriptional terminator. The transcriptional promoters cloned in front (5) of each gene were constitutive and derived from P. kudriavzevii. The transcriptional terminators cloned behind (3) of each gene were derived from S. cerevisiae. The promoter and terminator for RcMCL1 was the P. kudriavzevii PGK1 promoter (pPkPGK1) and the S. cerevisiae HXT1 terminator (tScHXT1), respectively. The promoter and terminator for MeMCSB was the P. kudriavzevii ENO1 promoter (pPkENO1) and the S. cerevisiae TEF1 terminator (tScTEF1), respectively. The promoter and terminator for MeMCSA was the P. kudriavzevii FBA1 promoter (pPkFBA1) and the S. cerevisiae GRE3 terminator (tScGRE3), respectively. The promoter and terminator for AtGLYR was the P. kudriavzevii TDH1 promoter (pPkTDH1) and the S. cerevisiae TPI terminator (tScTPI), respectively. Additionally, a H1S3 marker was included in the heterologous expression cassette to complement the histidine auxotrophic deficiency in the parent strain. This HIS3 marker comprised a transcriptional promoter, a HIS3 coding region, and a transcriptional terminator. The transcriptional promoter 5′ of HIS3 was the P. kudriavzevii TEF1 promoter (pPkTEF1) and the transcriptional terminator 3′ of HIS3 was the S. cerevisiae TDH3 terminator (tScTDH3).

) Each gene was amplified from its respective pEV vector using primers with upstream and downstream homologous regions to neighboring genetic elements to drive correct assembly of the full-length pathway. The upstream and downstream homologous regions were 25 bp to 700 bp in length. The 5′ and 3′ ends of the expression cassette comprised regions homologous to the genomic sequences upstream and downstream of the S. pombe MAE1 (from Example 4), thereby facilitating integration of the heterologous nucleic acids encoding the 3 remaining glycolic acid pathway enzymes (i.e., malate-CoA ligase, malyl-CoA lyase, and glyoxylate reductase) and into the S. pombe MAE1 site. As a consequence, the SpMAE1 malate transporter was removed and malate was no longer transported to the fermentation broth, which was useful in Example 5 for measuring malate production by HPLC.

All PCR products were purified and provided as exogenous nucleic acids to P. kudriavzevii. Transformation was carried out in a single step. Transformants were selected on CSM-His medium. Successful integration of all heterologous nucleic acids encoding the last 3 glycolic acid pathway enzymes as well as deletion of both copies of the genes encoding PkMAE1 were confirmed by genetic sequencing of this locus and the flanking regions.

Example 7 produced recombinant host cells LPK154945 that comprise heterologous nucleic acids encoding all S enzymes of the glycolic acid pathway of the present disclosure and the transporter SpMAE1, and further comprised genetic disruption of PkPDC1, PkPDC5, PkPDC6, PkPDA1, PkGPD1, and PkMAE1. LPK154945 was additionally auxotrophic for uracil.

Various publications were referenced in this application. The disclosures of these publications in their entireties are hereby incorporated by reference in this application in order to more fully describe the state of the art to which this disclosure pertains.

It should be noted that there are alternative ways of implementing the embodiments disclosed herein. Accordingly, the present embodiments are to be considered as illustrative and not restrictive; various modifications can be made without departing from the spirit of the present disclosure. Furthermore, the claims are not to be limited to the details given herein, and are entitled their full scope and equivalents thereof.

What is claimed is:

1. A recombinant host cell comprising a glycolic acid biosynthetic pathway: wherein the glycolic acid biosynthetic pathway comprises heterologous nucleic acids encoding an oxaloacetate-forming enzyme, a malate dehydrogenase, a malate-CoA ligase, a malyl-CoA lyase, and a glyoxyate reductase;
wherein the heterologous nucleic acids are expressed in sufficient amounts to produce glycolic acid.

2. The recombinant host cell of claim 1, wherein the recombinant host cell is a yeast cell.

3. The recombinant host cell of claim 2, wherein the yeast cell is of the Issatchenkia orientalis/Pichia fermentans clade.

4. The recombinant host cell of claim 2 or claim 3, wherein the yeast cell belongs to the genus Pichia, Issatchenkia or Candida.

5. The recombinant host cell of any one of claims 2-4, wherein the yeast cell is Pichia kudriavzevii.

6. The recombinant host cell of claim 2, wherein the yeast cell is of the Saccharomyces clade.

7. The recombinant host cell of claim 2 or claim 6, wherein the yeast cell is Saccharomyces cerevisiae.

8. The recombinant host cell of claim 1, wherein the recombinant host cell is a prokaryotic cell.

9. The recombinant host cell of claim 8, wherein the prokaryotic cell belongs to a genus selected from a group comprising Escherichia, Corynebacterium, Bacillus, and Lactococcus.

10. The recombinant host cell of claim 8 or claim 9, wherein the prokaryotic cell is selected from a group comprising Escherichia coli, Corynebacterium glutamicum, Bacillus subtilis, and Lactococcus lactis.

11. The recombinant host cell of claim 1, wherein the oxaloacetate-forming enzyme is selected from a group comprising pyruvate carboxylase, phosphoenolpyruvate carboxykinase, and phosphoenolpyruvate carboxylase.

12. The recombinant host cell of claim 11, wherein the pyruvate carboxylase has at least 60% homology to SEQ ID NO: 1, SEQ ID NO: 2, SEQ ID NO: 8, SEQ ID NO: 9, SEQ ID NO: 10, or SEQ ID NO: 53.

13. The recombinant host cell of claim 11, wherein the pyruvate carboxylase is selected from a group comprising SEQ ID NO: 1, SEQ ID NO: 2, SEQ ID NO: 8, SEQ ID NO: 9, SEQ ID NO: 10, and SEQ ID NO: 53.

14. The recombinant host cell of claim 11, wherein the phosphoenolpyruvate carboxykinase has at least 60% homology to SEQ ID NO: 54 or SEQ ID NO: 55.

15. The recombinant host cell of claim 11, wherein the phosphoenolpyruvate carboxykinase is selected from a group comprising SEQ ID NO: 54 and SEQ ID NO: 55.

16. The recombinant host cell of claim 11, wherein the phosphoenolpyruvate carboxylase has at least 60% homology to SEQ ID NO: 57.

17. The recombinant host cell of claim 11, wherein the phosphoenolpyruvate carboxylase is selected from a group comprising SEQ ID NO: 58, SEQ ID NO: 59, and SEQ ID NO: 60.

18. The recombinant host cell of claim 1, wherein the malate dehydrogenase has at least 60% homology to SEQ ID NO: 4, SEQ ID NO: 64, SEQ ID NO: 70, or SEQ ID NO: 71.

19. The recombinant host cell of claim 1, wherein the malate dehydrogenase is selected from a group comprising SEQ ID NO: 4, SEQ ID NO: 64, SEQ ID NO: 70, and SEQ ID NO: 71.

20. The recombinant host cell of claim 1, wherein the malate-CoA ligase has at least 60% homology to SEQ ID NO:6 and SEQ ID NO: 7, SEQ ID NO: 11 and SEQ JD NO: 12, SEQ ID NO: 13 and SEQ ID NO: 14, SEQ ID NO: 15 and SEQ ID NO: 16, SEQ ID NO: 63 and SEQ ID NO: 3, or SEQ ID NO: 56 and SEQ ID NO: 52.

21. The recombinant host cell of claim 1, wherein the malate-CoA ligase is selected from a group comprising SEQ ID NO: 6 and SEQ ID NO: 7, SEQ ID NO: 11 and SEQ ID NO: 12, SEQ ID NO: 13 and SEQ ID NO: 14, SEQ ID NO: 15 and SEQ ID NO: 16, SEQ ID NO: 63 and SEQ ID NO: 3, or SEQ ID NO: 56 and SEQ ID NO: 52.

22. The recombinant host cell of claim 1, wherein the malyl-CoA lyase has at least 40% homology to SEQ ID NO: 21.

23. The recombinant host cell of claim 1, wherein the malyl-CoA lyase is selected from a group comprising SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30.

24. The recombinant host cell of claim 1, wherein the glyoxylate reductase has at least 60% homology to SEQ ID NO: 22.

25. The recombinant host cell of claim 1, wherein the glyoxylate reductase is selected from a group comprising SEQ ID NO: 5, SEQ ID NO: 17, SEQ ID NO: 18, SEQ ID NO: 19, SEQ ID NO: 20, SEQ ID NO: 31, SEQ ID NO: 32, SEQ ID NO: 33, SEQ ID NO: 34, SEQ ID NO: 66, SEQ ID NO: 80, SEQ ID NO: 81, SEQ ID NO: 82, SEQ ID NO: and 83 SEQ ID NO: 84.

26. The recombinant host cell of claim 1, further comprising:
one or more heterologous nucleic acids encoding one or more ancillary proteins;
wherein the one or more ancillary proteins function in redox cofactor recycling, redox cofactor biogenesis, organic acid transport, carbon fixation, or improving flux through the glycolic acid biosynthetic pathway.

27. The recombinant host cell of claim 26, wherein the one or more ancillary proteins is a glycolic acid transporter, a carbon fixation enzyme, or a combination thereof.

28. The recombinant host cell of claim 27, wherein the glycolic acid transporter has at least 60% amino acid homology to SEQ ID NO: 35, SEQ ID NO: 36, SEQ ID NO: 37, SEQ ID NO: 38, SEQ ID NO: 65, SEQ ID NO: 67, SEQ ID NO: 76, SEQ ID NO: 77, SEQ ID NO: 78, or SEQ ID NO: 79.

29. The recombinant host cell of claim 27, wherein the glycolic acid transporter is selected from a group comprising SEQ ID NO: 35, SEQ ID NO: 36, SEQ ID NO: 37, SEQ ID NO: 38, SEQ ID NO: 65, SEQ ID NO: 67, SEQ ID NO: 76, SEQ ID NO: 77, SEQ ID NO: 78, and SEQ ID NO: 79.

30. The recombinant host cell of claim 27, wherein the carbon fixation enzyme is a carbonic anhydrase.

31. The recombinant host cell of claim 27, wherein the carbon fixation enzyme has at least 60% homology to SEQ ID NO: 44, SEQ ID NO: 45, SEQ ID NO: 46, SEQ ID NO: 47, SEQ ID NO: 48, SEQ ID NO: 49, SEQ ID NO: 50, or SEQ ID NO: 51.

32. The recombinant host cell of claim 27, wherein the carbon fixation enzyme is selected from a group comprising SEQ ID NO: 44, SEQ ID NO: 45, SEQ ID NO: 46, SEQ ID NO: 47, SEQ ID NO: 48, SEQ ID NO: 49, SEQ ID NO: 50, and SEQ ID NO: 51.

33. The recombinant host cell of claim 1, further comprising a genetic disruption of one or more genes, wherein
the one or more genes encodes pyruvate decarboxylase, pyruvate dehydrogenase complex, glycerol-3-phosphate dehydrogenase, malate synthase, glycine dehydrogenase, or a combination thereof.

34. The recombinant host cell of claim 33, wherein the one or more genes has at least 60% amino acid homology to SEQ ID NO: 39, SEQ ID NO: 40, or SEQ ID NO: 41.

35. The recombinant host cell of claim 34, wherein the recombinant host cell produces less than 5 g/l ethanol.

36. The recombinant host cell of claim 33, wherein the one or more genes has at least 60% amino acid homology to SEQ ID NO: 42.

37. The recombinant host cell of claim 36, wherein the recombinant host cell produces less carbon dioxide than a recombinant host cell that does not comprise said genetic disruption of a gene with at least 60% amino acid homology to SEQ ID NO: 42.

38. The recombinant host cell of claim 33, wherein the one or more genes has at least 60% amino acid homology to SEQ ID NO: 43.

39. The recombinant host cell of claim 38, wherein the recombinant host cell produces less than 10 g/l glycerol.

40. The recombinant host cell of claim 33, wherein the one or more genes has at least 60% amino acid homology to SEQ ID NO: 72, SEQ ID NO: 73, or SEQ ID NO: 74.

41. The recombinant host cell of claim 40, wherein the recombinant host cell produces less than 10 g/l malate.

42. The recombinant host cell of claim 33, wherein the one or more genes has at least 60% amino acid homology to SEQ ID NO: 75.

43. A method for the production of glycolic acid comprising the steps of:
culturing the recombinant host cell of claim 1 in fermentation broth for a sufficient period of time to produce glycolic acid.

44. The method of claim 43, wherein the culturing is performed at an oxygen transfer rate that is greater than 10 mmol/l/hr.

45. The method of claim 43 or claim 44, wherein the culturing is performed at a temperature of between about 25° C. and about 45° C.

46. The method of any one of claims 43-45, wherein the culturing is performed such that the final fermentation pH of between pH 4 and pH 8.

47. The method of any one of claims 43-46, further comprising carbon dioxide supplementation.

48. The method of any one of claims 43-47, further comprising bicarbonate supplementation.

49. The method of any one of claims 43-48, further comprising producing at least 25 g/l glycolic acid in the fermentation broth.

50. The method of any one of claims 43-49, further comprising
providing at least 100 g/l glucose to the recombinant host cells; and
producing a glycolic acid yield of at least 25%.


SEQUENCE LISTING (84 sequences)

SEQ ID NO: 1 - protein, 1180 aa, Saccharomyces cerevisiae
MSSSKKLAGLRDNFSLLGEKNKILVANRGEIPIRIFRSAHELSMRTIAIYSHEDRLSMHRLKADEAYVIGEEGQYTPVGAYLAMDEIIEIAKKHKVDFIHPGYGFLSENSEFADKVVKAGITWIGPPAEVIDSVGDKVSARHLAARANVPTVPGTPGPIETVQEALDFVNEYGYPVIIKAAFGGGGRGMRVVREGDDVADAFQRATSEARTAFGNGTCFVERFLDKPKHIEVQLLADNHGNVVHLFERDCSVQRRHQKVVEVAPAKTLPREVRDAILTDAVKLAKVCGYRNAGTAEFLVDNQNRHYFIEINPRIQVEHTITEEITGIDIVSAQIQIAAGATLTQLGLLQDKITTRGFSIQCRITTEDPSKNFQPDTGRLEVYRSAGGNGVRLDGGNAYAGATISPHYDSMLVKCSCSGSTYEIVRRKMIRALIEFRIRGVKTNIPFLLTLLTNPVFIEGTYWTTFIDDTPQLFQMVSSQNRAQKLLHYLADLAVNGSSIKGQIGLPKLKSNPSVPHLHDAQGNVINVTKSAPPSGWRQVLLEKGPSEFAKQVRQFNGTLLMDTTWRDAHQSLLATRVRTHDLATIAPTTAHALAGAFALECWGGATFDVAMRFLHEDPWERLRKLRSLVPNIPFQMLLRGANGVAYSSLPDNAIDHFVKQAKDNGVDIFRVFDALNDLEQLKVGVNAVKKAGGVVEATVCYSGDMLQPGKKYNLDYYLEVVEKIVQMGTHILGIKDMAGTMKPAAAKLLIGSLRTRYPDLPIHVHSHDSAGTAVASMTACALAGADVVDVAINSMSGLTSQPSINALLASLEGNIDTGINVEHVRELDAYWAEMRLLYSCFEADLKGPDPEVYQHEIPGGQLTNLLFQAQQLGLGEQWAETKRAYREANYLLGDIVKVTPTSKVVGDLAQFMVSNKLTSDDIRRLANSLDFPDSVMDFFEGLIGQPYGGFPEPLRSDVLRNKRRKLTCRPGLELEPFDLEKIREDLQNRFGDIDECDVASYNMYPRVYEDFQKIRETYGDLSVLPTKNFLAPAEPDEEIEVTIEQGKTLIIKLQAVGDLNKKTGQREVYFELNGELRKIRVADKSQNIQSVAKPKADVHDTHQIGAPMAGVIIEVKVHKGSLVKKGESIAVLSAMKMEMVVSSPADGQVKDVFIKDGESVDASDLLVVLEEETLPPSQKK

SEQ ID NO: 2 - protein, 1178 aa, Saccharomyces cerevisiae
MSQRKFAGLRDNFNLLGEKNKILVANRGEIPIRIFRTAHELSMQTVAIYSHEDRLSTHKQKADEAYVIGEVGQYTPVGAYLAIDEIISIAQKHQVDFIHPGYGFLSENSEFADKVVKAGITWIGPPAEVIDSVGDKVSARNLAAKANVPTVPGTPGPIETVEEALDFVNEYGYPVIIKAAFGGGGRGMRVVREGDDVADAFQRATSEARTAFGNGTCFVERFLDKPKHIEVQLLADNHGNVVHLFERDCSVQRRHQKVVEVAPAKTLPREVRDAILTDAVKLAKECGYRNAGTAEFLVDNQNRHYFIEINPRIQVEHTITEEITGIDIVAAQIQIAAGASLPQLGLFQDKITTRGFAIQCRITTEDPAKNFQPDTGRIEVYRSAGGNGVRLDGGNAYAGTIISPHYDSMLVKCSCSGSTYEIVRRKMIRALIEFRIRGVKTNIPFLLTLLTNPVFIEGTYWTTFIDDTPQLFQMVSSQNRAQKLLHYLADVAVNGSSIKGQIGLPKLKSNPSVPHLHDAQGNVINVTKSAPPSGWRQVLLEKGPAEFARQVRQFNGTLLMDTTWRDAHQSLLATRVRTHDLATIAPTTAHALAGRFALECWGGATFDVAMRFLHEDPWERLRKLRSLVPNIPFQMLLRGANGVAYSSLPDNAIDHFVKQAKDNGVDIFRVFDALNDLEQLKVGVDAVKKAGGVVEATVCFSGDMLQPGKKYNLDYYLEIAEKIVQMGTHILGIKDMAGTMKPAAAKLLIGSLRAKYPDLPIHVHTHDSAGTAVASMTACALAGADVVDVAINSMSGLTSQPSINALLASLEGNIDTGINVEHVRELDAYWAEMRLLYSCFEADLKGPDPEVYQHEIPGGQLTNLLFQAQQLGLGEQWAETKRAYREANYLLGDIVKVTPTSKVVGDLAQFMVSNKLTSDDVRRLANSLDFPDSVMDFFEGLIGQPYGGFPEPFRSDVLRNKRRKLTCRPGLELEPFDLEKIREDLQNRFGDVDECDVASYNMYPRVYEDFQKMRETYGDLSVLPTRSFLSPLETDEEIEVVIEQGKTLIIKLQAVGDLNKKTGEREVYFDLNGEMRKIRVADRSQKVETVTKSKADMHDPLHIGAPMAGVIVEVKVHKGSLIKKGQPVAVLSAMKMEMIISSPSDGQVKEVFVSDGENVDSSDLLVLLEDQVPVETKA

SEQ ID NO: 3 - protein, 394 aa, unidentified
MDIHEYQAKELLARHGVHVPRGGLAYSPEQATYRAREIGGGKWVLKAQVHSGARGKAGGIKLCANDEEISSAAEAMLGRKLVTQQTGPRGKLISRLYLEEAVDIAQELYVGFVLDRKEERVMIVASAAGGMEIEDIVEKEPNSILRTSVDPGVGMQRFQAREIAFGLGLDHNLIGKATETIFSCYQVFRDYDASMLEINPLVVTRDGNLIALDAKMSFDENALFRRPEISELRDKSQEDPRETFASDRGLSYVGLDGNIGCIINGAGLAMATMDMIKIAGGEPANFLDIGGGASPERVAKSFRAVLGDKNVETILVNIFAGINRCDWVAEGVIKAIREVGVNVPLVVRLSGTKAEEGRRILADSGEAVIVADTLAEAAEKAVAAWRAAAKKKAA

SEQ ID NO: 4 - protein, 340 aa, Saccharomyces cerevisiae
MVKVAILGASGGVGQPLSLLLKLSPYVSELALYDIRAAEGIGKDLSHINTNSSCVGYDKDSIENTLSNAQVVLIPAGVPRKPGLTRDDLFKMNAGIVKSLVTAVGKFAPNARILVISNPVNSLVPIAVETLKKMGKFKPGNVMGVTNLDLVRAETFLVDYLMLKNPKIGQEQDKTTMHRKVTVIGGHSGETIIPIITDKSLVFQLDKQYEHFIHRVQFGGDEIVKAKQGAGSATLSMAFAGAKFAEEVLRSFHNEKPETESLSAFVYLPGLKNGKKAQQLVGDNSIEYFSLPIVLRNGSVVSIDTSVLEKLSPREEQLVNTAVKELRKNIEKGKSFILDS

SEQ ID NO: 5 - protein, 312 aa, Escherichia coli
MDIIFYHPVFDTAWWIDALGKAIPQARVRAWKTGDNHPADYALVWHPPVEMLAGRDLKAVFALGAGVDSILSKLQTYPEMLKPSIPLFRLEDTGMGEQMQEYAVSQVLHWFRRFDDYRAQQNASLWQPLPEYQREDFTIGILGAGVLGSKVAQSLQTWCFPLRCWSRSHKSWPGVQSFAGTEELGKFLSQCRVLINLLPNTPETVGIINQQLLEKLPDGAYLLNLSRGVHVVEDDLLAALDSGKVKAAMLDVFNREPLPPESPLWQHPRVTITPHVAAITRPVEAVEYIAHTITQLEKGKTVSGQVDRVRGY

SEQ ID NO: 6 - protein, 293 aa, Advenella mimigardefordensis
MSILINKDSKVITQGITGKTGQFHTRMCRDYANGKEAFVAGVNPKRAGEDFEGVPIYASVKDAKEQTGANVSVIYVPPAGAADAIWEAVDADLDLVICITEGIPVRDMLAVRNRMRQENKKTLLLGPNCPGLITPDELKIGIMPAHICKKGRIGVVSRSGTLTYEAVAQLTELGLGQSSAVGIGGDPINGLKHIDILKMFNDDPETDAVVMIGEIGGPDEVNAAEWAKDNMSKPVVGFIAGVTAPPGKRMGHAGALISGGADTADAKLEIMEACGIRTTRNPSEMGKLLKSVL

SEQ ID NO: 7 - protein, 386 aa, Advenella mimigardefordensis
MKIHEYQGKELLKKFGVTVPRGIPAFSVDEAVAAAEKLGGPVWVVKAQIHAGGRGKGGGVKLAKSIDEVRQLSSEILGMQLVTHQTGPEGQKVNRLLIEEGADIKKEYYVGIVTDRSSQRVCVMASSEGGMDVEEVAEKTPEKILKVFVDPAKGFTDDEARELARGIGVPDASVEKAAVEFQKLYQAYWDTDSSLAEINPLILTGSGDIIALDAKFNFDSNALFRHPEIVEYRDLDEEDPAEIEASKFDLAYIQLDGNIGCLVNGAGLAMATMDTIKLFGGEPANFLDVGGGATAEKVTEAFKIMLKNKEVKAILVNIFGGIMRCDVIAEGVIAACKAVNLSVPLVVRMKGTNEEMGKKMLAESGLPIISADTMAEAATKVVAAVK

SEQ ID NO: 8 - protein, 1180 aa, unidentified
MSTVEDHSSLHKLRKESEILSNANKILVANRGEIPIRIFRSAHELSMHTVAIYSHEDRLSMHRLKADEAYAIGKTGQYSPVQAYLQIDEIIKIAKEHDVSMIHPGYGFLSENSEFAKKVEESGMIWVGPPAEVIDSVGDKVSARNLAIKCDVPVVPGTDGPIEDIEQAKQFVEQYGYPVIIKAAFGGGGRGMRVVREGDDIVDAFQRASSEAKSAFGNGTCFIERFLDKPKHIEVQLLADNYGNTIHLFERDCSVQRRHQKVVEIAPAKTLPVEVRNAILKDAVTLAKTANYRNAGTAEFLVDSQNRHYFIEINPRIQVEHTITEEITGVDIVAAQIQIAAGASLEQLGLLQNKITTRGFAIQCRITTEDPAKNFAPDTGKIEVYRSAGGNGVRLDGGNGFAGAVISPHYDSMLVKCSTSGSNYEIARRKMIRALVEFRIRGVKTNIPFLLALLTHPVFISGDCWTTFIDDTPSLFEMVSSKNRAQKLLAYIGDLCVNGSSIKGQIGFPKLNKEAEIPDLLDPNDEVIDVSKPSTNGLRPYLLKYGPDAFSKKVRGFDGCMIMDTTWRDAHQSLLATRVRTIDLLRIAPTTSHALQNAFALECWGGATFDVAMRFLYEDPWERLRQLRKAVPNIPFQMLLRGANGVAYSSLPDNAIDHFVKQAKDNGVDIFRVFDALNDLEQLKVGVDAVKKAGGVVEATVCYSGDMLIPGKKYNLDYYLETVGKIVEMGTHILGIKDMAGTLKPKAAKLLIGSIRSKYPDLVIHVHTHDSAGTGISTYVACALAGADIVDCAINSMSGLTSQPSMSAFIAALDGDIETGVPEHFARQLDAYWAEMRLLYSCFEADLKGPDPEVYKHEIPGGQLTNLIFQAQQVGLGEQWEETKKKYEDANMLLGDIVKVTPTSKVVGDLAQFMVSNKLEKEDVEKLANELDFPDSVLDFFEGLMGTPYGGFPEPLRTNVISGKRRKLKGRPGLELEPFNLEEIRENLVSRFGPGITECDVASYNMYPKVYEQYRKVVEKYGDLSVLPTKAFLAPPTIGEEVHVEIEQGKTLIIKLLAISDLSKSHGTREVYFELNGEMRKVTIEDKTAAIETVTRAKADGHNPNEVGAPMAGVVVEVRVKHGTEVKKGDPLAVLSAMKMEMVISAPVSGRVGEVFVNEGDSVDMGDLLVKIAKDEAPAA

SEQ ID NO: 9 - protein, 1180 aa, unidentified
MSTVEDHSSLHKLRKESEILSNANKILVANRGEIPIRIFRSAHELSMHTVAIYSHEDRLSMHRLKADEAYAIGKTGQYSPVQAYLQIDEIIKIAKEHDVSMIHPGYGFLSENSEFAKKVEESGMIWVGPPAEVIDSVGDKVSARNLAIKCDVPVVPGTDGPIEDIEQAKQFVEQYGYPVIIKAAFGGGGRGMRVVREGDDIVDAFQRASSEAKSAFGNGTCFIERFLDKPKHIEVQLLADNYGNTIHLFERDCSVQRRHQKVVEIAPAKTLPVEVRNAILKDAVTLAKTANYRNAGTAEFLVDSQNRHYFIEINPRIQVEHTITEEITGVDIVAAQIQIAAGASLEQLGLLQNKITTRGFAIQCRITTEDPAKNFAPDTGKIEVYRSAGGNGVRLDGGNGFAGAVISPHYDSMLVKCSTSGSNYEIARRKMIRALVEFRIRGVKTNIPFLLALLTHPVFISGDCWTTFIDDTPSLFEMVSSKNRAQKLLAYIGDLCVNGSSIKGQIGFPKLNKEAEIPDLLDPNDEVIDVSKPSTNGLRPYLLKYGPDAFSKKVREFDGCMIMDTTWRDAHQSLLATRVRTIDLLRIAPTTSHALQNAFALECWGGATFDVAMRFLYEDPWERLRQLRKAVPNIPFQMLLRGANGVAYSSLPDNAIDHFVKQAKDNGVDIFRVFDALNDLEQLKVGVDAVKKAGGVVEATVCYSGDMLIPGKKYNLDYYLETVGKIVEMGTHILGIKDMAGTLKPKAAKLLIGSIRSKYPDLVIHVHTHDSAGTGISTYVACALAGADIVDCAINSMSGLTSQPSMSAFIAALDGDIETGVPEHFARQLDAYWAEMRLLYSCFEADLKGPDPEVYKHEIPGGQLTNLIFQAQQVGLGEQWEETKKKYEDANMLLGDIVKVTPTSKVVGDLAQFMVSNKLEKEDVEKLANELDFPDSVLDFFEGLMGTPYGGFPEPLRTNVISGKRRKLKGRPGLELEPFNLEEIRENLVSRFGPGITECDVASYNMYPKVYEQYRKVVEKYGDLSVLPTKAFLAPPTIGEEVHVEIEQGKTLIIKLLAISDLSKSHGTREVYFELNGEMRKVTIEDKTAAIETVTRAKADGHNPNEVGAPMAGVVVEVRVKHGTEVKKGDPLAVLSAMKMEMVISAPVSGRVGEVFVNEGDSVDMGDLLVKIAKDEAPAA

SEQ ID NO: 10 - protein, 1143 aa, unidentified
MSTILSNANKILVANRGEIPIRIFRSAHELSMHTVAIYSHEDRLSMHRLKADEAYAIGKTGQYSPVQAYLQIDEIIKIAKEHDVSMIHPGYGFLSENSEFAKKVEESGMIWVGPPAEVIDSVGDKVSARNLAIKCDVPVVPGTDGPIEDIEQAKQFVEQYGYPVIIKAAFGGGGRGMRVVREGDDIVDAFQRASSEAKSAFGNGTCFIERFLDKPKHIEVQLLADNYGNTIHLFERDCSVQRRHQKVVEIAPAKTLPVEVRNAILKDAVTLAKTANYRNAGTAEFLVDSQNRHYFIEINPRIQVEHTITEEITGVDIVAAQIQIAAGASLEQLGLLQNKITTRGFAIQCRITTEDPAKNFAPDTGKIEVYRSAGGNGVRLDGGNGFAGAVISPHYDSMLVKCSTSGSNYEIARRKMIRALVEFRIRGVKTNIPFLLALLTHPVFISGDCWTTFIDDTPSLFEMVSSKNRAQKLLAYIGDLCVNGSSIKGQIGFPKLNKEAEIPDLLDPNDEVIDVSKPSTNGLRPYLLKYGPDAFSKKVRGFDGCMIMDTTWRDAHQSLLATRVRTIDLLRIAPTTSHALQNAFALECWGGATFDVAMRFLYEDPWERLRQLRKAVPNIPFQMLLRGANGVAYSSLPDNAIDHFVKQAKDNGVDIFRVFDALNDLEQLKVGVDAVKKAGGVVEATVCYSGDMLIPGKKYNLDYYLETVGKIVEMGTHILGIKDMAGTLKPKAAKLLIGSIRSKYPDLVIHVHTHDSAGTGISTYVACALAGADIVDCAINSMSGLTSQPSMSAFIAALDGDIETGVPEHFARQLDAYWAEMRLLYSWTRPEVYKHEIPGGQLTNLIFQAQQVGLGEQWEETKKKYEDANMLLGDIVKVTPTSKVVGDLAQFMVSNKLEKEDVEKLANELDFPDSVLDFFEGLMGTPYGGFPEPLRTNVISGKRRKLKGRPGLELEPFNLEEIRENLVSRFGPGITECDVASYNMYPKVYEQYRKVVEKYGDLSVLPTKAFLAPPTIGEEVHVEIEQGKTLIIKLLAISDLSKSHGTREVYFELNGEMRKSKGDGHNPNEVGAPMAGVVVEVRVKHGTEVKKGDPLAVLSAMKMEMVISAPVSGRVGEVFVNEGDSVDMGDLLVKIAKDEAPAA

SEQ ID NO: 11 - protein, 389 aa, Alcanivorax borkumensis
MNLHEYQSKQLFADYGLPVSTGFACDTPEEVAAKAKEIGGDKWVVKAQVHAGGRGKAGGVKLVDTPEDAAAFAKKWLGKQLVTFQTDEHGQPVSKILVETCTDIAKELYLSAVLDRASRRVVFMASTEGGVDIETVAEETPEKILKAEIDPLVGAQPYQAREIAFKLGLEGKQVNQFAKIFVGLAKLFVDKDLALIEVNPLVITDTGDLHCLDAKINVDGSALYRHPDIKALDDPSQEDERERRAAEWDLNYVALEGNIGCMVNGAGLAMGTMDLVKLKGGAPANFLDVGGGATKERVTEAFKIILSDDQVKGVLVNIFGGIVRCDLIAEGIIGAVEEVGVTVPVVVRLEGNNAELGSQKLAESGMNIIAAQSFDDAAEQVVKAVGGAA

SEQ ID NO: 12 - protein, 291 aa, Alcanivorax borkumensis
MSVLIDKNTKVICQGFTGSQGTLHSEQAIEYGTQMVGGVTPGKGGQTHLGLPVFNTVAEAVAETGATASVIYVPAPFCKDSILEAADAGIELIVCITEGVPALDMLFVKEYISRKDGIRLIGPNCPGVITPGECKIGIMPGHIHTPGKVGIVSRSGTLTYEAVNQTTKLGFGQSSCIGIGGDPIPGMTFIDALELFQNDPQTEAIVMVGEIGGTAEDEAAAYIKEHVTKPVVSYIAGVTAPAGKRMGHAGAIIAGGKGTADEKFAVLEAAGVKTVRNPAEIGKAVKEATGW

SEQ ID NO: 13 - protein, 388 aa, Escherichia coli
MNLHEYQAKQLFARYGLPAPVGYACTTPREAEEAASKIGAGPWVVKCQVHAGGRGKAGGVKVVNSKEDIRAFAENWLGKRLVTYQTDANGQPVNQILVEAATDIAKELYLGAVVDRSSRRVVFMASTEGGVEIEKVAEETPHLIHKVALDPLTGPMPYQGRELAFKLGLEGKLVQQFTKIFMGLATIFLERDLALIEINPLVITKQGDLICLDGKLGADGNALFRQPDLREMRDQSQEDPREAQAAQWELNYVALDGNIGCMVNGAGLAMGTMDIVKLHGGEPANFLDVGGGATKERVTEAFKIILSDDKVKAVLVNIFGGIVRCDLIADGIIGAVAEVGVNVPVVVRLEGNNAELGAKKLADSGLNIIAAKGLTDAAQQVVAAVEGK

SEQ ID NO: 14 - protein, 289 aa, Escherichia coli
MSILIDKNTKVICQGFTGSQGTFHSEQAIAYGTKMVGGVTPGKGGTTHLGLPVFNTVREAVAATGATASVIYVPAPFCKDSILEAIDAGIKLIITITEGIPTLDMLTVKVKLDEAGVRMIGPNCPGVITPGECKIGIQPGHIHKPGKVGIVSRSGTLTYEAVKQTTDYGFGQSTCVGIGGDPIPGSNFIDILEMFEKDPQTEAIVMIGEIGGSAEEEAAAYIKEHVTKPVVGYIAGVTAPKGKRMGHAGAIIAGGKGTADEKFAALEAAGVKTVRSLADIGEALKTVLK

SEQ ID NO: 15 - protein, 390 aa, Methylobacterium extorquens
MDVHEYQAKELLASFGVAVPKGAVAFSPDQAVYAATELGGSFWAVKAQIHAGARGKAGGIKLCRTYNEVRDAARDLLGKRLVTLQTGPEGKPVQRVYVETADPFERELYLGYVLDRKAERVRVIASQRGGMDIEEIAAKEPEALIQVVVEPAVGLQQFQAREIAFQLGLNIKQVSAAVKTIMNAYRAFRDCDGTMLEINPLVVTKDDRVLALDAKMSFDDNALFRRRNIADMHDPSQGDPREAQAAEHNLSYIGLEGEIGCIVNGAGLAMATMDMIKHAGGEPANFLDVGGGASPDRVATAFRLVLSDRNVKAILVNIFAGINRCDWVAEGVVKAAREVKIDVPLIVRLAGTNVDEGKKILAESGLDLITADTLTEAARKAVEACHGAKH

SEQ ID NO: 16 - protein, 296 aa, Methylobacterium extorquens
MSILIDEKTPILVQGITGDKGTFHAKEMIAYGSNVVGGVTPGKGGKTHCGVPVFNTVKEAVEATGATTSITFVAPPFAADAIMEAADAGLKLVCSITDGIPAQDMMRVKRYLRRYPKEKRTMVVGPNCAGIISPGKSMLGIMPGHIYLPGKVGVISRSGTLGYEAAAQMKELGIGISTSVGIGGDPINGSSFLDHLALFEQDPETEAVLMIGEIGGPQEAEASAWIKENFSKPVIGFVAGLTAPKGRRMGHAGAIISATGDSAAEKAEIMRSYGLTVAPDPGSFGSTVADVLARAA

SEQ ID NO: 17 - protein, 323 aa, Klebsiella pneumoniae
MKPSVILYKTLPDDLLQRLEEHFSVTQVKNLRPETVSQHAEAFAQAEGLLGSSEKVDAALLEKMPKLRATSTVSVGYDNFDVEALNARRVLLMHTPTVLTETVADTVMALVLSTARRVVEVAERVKAGEWTKSIGPDWFGSDVHHKTLGIVGMGRIGMALAQRAHFGFGMPILYNARRQHPQAEERFNARYCDLDTLLQEADFVCLILPLSEETHHLFGQAQFAKMKSSAIFINAGRGPVVDEQALIAALQAGEIHAAGLDVFEHEPLAKDSPLLTLPNVVALPHIGSATHETRYNMAACAVDNLIDALNGNVEKNCVNPQVK

SEQ ID NO: 18 - protein, 300 aa, Sulfolobus solfataricus
MKIISTEKVPDECKNIISVKDENITEEDYKNAEILLTWPGRVNNDLIGKMPNLKVIQTFSAGVDDLDFSIIPSHVKVFSNAGAYSLSVAEHAWALILASAKGVGTKKRTIVYDVSEKTLLILGGGGIGSEVARIGKTAFRNYVIGISRSFKKPEWFDERHGMTMLREKIGEADIIVDTLPLNKQTRGLLSYDLLKDIRRNAIIVNVGRGETVDEEGIYKLLKERQDVRFGTDVFWRKNGKEDFYNTKLWELDNFTGTLHTAGAYGNESIMKRAMLIACLNVKKYIDRGIADNEVRREDYV

SEQ ID NO: 19 - protein, 289 aa, Arabidopsis thaliana
MEVGFLGLGIMGKAMSMNLLKNGFKVTVWNRTLSKCDELVEHGASVCESPAEVIKKCKYTIAMLSDPCAALSVVFDKGGVLEQICEGKGYIDMSTVDAETSLKINEAITGKGGRFVEGPVSGSKKPAEDGQLIILAAGDKALFEESIPAFDVLGKRSFYLGQVGNGAKMKLIVNMIMGSMMNAFSEGLVLADKSGLSSDTLLDILDLGAMTNPMFKGKGPSMNKSSYPPAFPLKHQQKDMRLALALGDENAVSMPVAAAANEAFKKARSLGLGDLDFSAVIEAVKFSRE

SEQ ID NO: 20 - protein, 319 aa, Rhizobium etli
MSTPPPVLVDIKFNPEGVTRVLKTAFADRGSINLADPADRLRDLSAAEYALLWKPDADLFQRAPNLKVIFSGGAGVDHIIGMDSLPEIPIVRFVDRSLTTRMSEWVVMQSLMHLRGQYAHDISQRRGEWAKLIAPEAAEVTVGVMGLGVLGQDAVTKLKVMGFNVIGWSRTRKEIDGVETFDARELDAFLARTDILVGLLPLTPDTTGFYDSGLFKKLRRDGALGRPVFINAGGGRSQVEADIVSAIRDGILGGASLDVFEVEPLASDNPLWALENVFITPHDAAVSEENALFRHVETQIARFERGEPLQFVVDRAAGY

SEQ ID NO: 21 - protein, 322 aa, synthetic construct
MSFRLQPEAPARPNRCQLFGPGSNPKLFEKMAASAADVINLDLEDAVAPDDKEAARANIIEAINALDWGDKTLSVRINGLDTPWWYRDVVDLLEAAGDRLDQIMIPKVGCAADIYAVDALVTAIERAKGRKKPISFEVIIESAAGIAHVEAIAAASPRLEAMSLGAADFAASMGMKTTGIGGTQENYYMLRDPEGQFHWQDPWHWAQAAIVAACRAHGILPVDGPFGDFSDDEGFRAQARRAATLGMVGKWAIHPKQIALANEVFTPSEEAVAEAREILAAMEAAKADGEGATVYDGRLVDIASIKQAEVIVDQAEMIAGKS

SEQ ID NO: 22 - protein, 324 aa, synthetic construct
MKPSIILYKALPDDLLQRLEEHFTVTQVPNLRPETVEQHAEAFANAEGLLGSSEKVDAALLEKMPKLRATSTVSVGYDNFDVEALNARRVLLMHTPTVLTETVADTVMALVLSTARRVVEVAERVKAGEWTKSIGPDWFGTDVHHKTLGIVGMGRIGMALAQRAHFGFNMPILYNARRHHPQAEERFNARYCDLDTLLQEADFVCLILPLTEETHHLFGAAQFAKMKSSAIFINAGRGPVVDEDALIAALQSGEIHAAGLDVFEQEPLPVDSPLLSLPNVVALPHIGSATHETRYNMAACAVDNLIDALQGKVEKNCVNPQVAG

SEQ ID NO: 23 - protein, 318 aa, Rhodobacter sphaeroides
MSFRLQPAPPARPNRCQLFGPGSRPALFEKMAASAADVINLDLEDSVAPDDKAQARANIIEAINGLDWGRKYLSVRINGLDTPFWYRDVVDLLEQAGDRLDQIMIPKVGCAADVYAVDALVTAIERAKGRTKPLSFEVIIESAAGIAHVEEIAASSPRLQAMSLGAADFAASMGMQTTGIGGTQENYYMLHDGQKHWSDPWHWAQAAIVAACRTHGILPVDGPFGDFSDDEGFRAQARRSATLGMVGKWAIHPKQVALANEVFTPSETAVTEAREILAAMDAAKARGEGATVYKGRLVDIASIKQAEVIVRQAEMISA

SEQ ID NO: 24 - protein, 318 aa, Rhodobacter capsulatus
MSFRTQPPAPARLNRCQLFGPGSRPAIFEKMAQSAADVINLDLEDSVAPDDKPQARRNIIEASHNIDWGNKYLSVRINGLDTPFWYRDVVELLEDGSERIDQIMIPKVGCAADVYAVDALVTAIEAAKGRKKRISLEVIIESAAGIAHVEEIAAASPRLQAMSLGAADFAASMGMATTGIGGTQENYYMLHAGVKHWSDPWHWAQAAIVAACRTHGILPVDGPFGDFSDDEGFRAQALRSATLGMVGKWAIHPKQVALANEVFTPSDAAVAEAREILAAMEKAKAEGAGATVYKGRLVDIASIRQAEVIVRQAEMAKV

SEQ ID NO: 25 - protein, 348 aa, Chloroflexus aurantiacus
MRKLAHNFYKPLAIGAPEPIRELPVRPERVVHFFPPHVEKIRARIPEVAKQVDVLCGNLEDAIPMDAKEAARNGFIEVVKATDFGDTALWVRVNALNSPWVLDDIAEIVAAVGNKLDVIMIPKVEGPWDIHFVDQYLALLEARHQIKKPILIHALLETAQGMVNLEEIAGASPRMHGFSLGPADLAASRGMKTTRVGGGHPFYGVLADPQEGQAERPFYQQDLWHYTIARMVDVAVAHGLRAFYGPFGDIKDEAACEAQFRNAFLLGCTGAWSLAPNQIPIAKRVFSPDVNEVLFAKRILEAMPDGSGVAMIDGKMQDDATWKQAKVIVDLARMIAKKDPDLAQAYGL

SEQ ID NO: 26 - protein, 324 aa, Methylobacterium extorquens
MSFTLIQQATPRLHRSELAVPGSNPTFMEKSAASKADVIFLDLEDAVAPDDKEQARKNIIQALNDLDWGNKTMMIRINGLDTHYMYRDVVDIVEACPRLDMILIPKVGVPADVYAIDVLTTQIEQAKKREKKIGFEVLIETALGMANVEAIATSSKRLEAMSFGVADYAASTRARSTVIGGVNADYSVLTDKDEAGNRQTHWQDPWLFAQNRMLVACRAYGLRPIDGPFGDFSDPDGYTSAARRCAALGFEGKWAIHPSQIDLANEVFTPSEAEVTKARRILEAMEEAAKAGRGAVSLDGRLIDIASIRMAEALIQKADAMGGK

SEQ ID NO: 27 - protein, 318 aa, Rhodobacter sp.
MSFRLQPPAPARPNRCQLFGPGSRPALFEKMAASAADVINLDLEDSVAPSDKDVARANIIEAISAVDWGRKTLSVRINGLDTPWWYRDVVDLLEKGGDRLDQIMIPKVGCAEDVYAVDALVTAVERAMGRVRPISFEVIIESAAGISHVEAIAASSPRLQAMSLGAADFAASMGMQTTGIGGTQENYYMLREGARHWSDPWHWAQAAIVAACRTHGILPVDGPFGDFSDDEGFRAQARRSATLGMVGKWAIHPKQVALANEVFTPSEQAVAEAREILAAMEAAKARGEGATVYKGRLVDIASIKQAEVIVRQAELIGG

SEQ ID NO: 28 - protein, 319 aa, Roseobacter sp.
MSFRLQPAAPARPNRCQLFGPGSNTKLFPKMAASAADVINLDLEDSVAPSDKDAARANVIEALNTVDWGNKYMSVRINGLDTPYWYRDVVDLLEQAGDRIDQIMIPKVGCAEDVYAVDALVTAIERAKGRSKPISFEVIIESAAGIAHVEEIAASSPRLQAMSLGAADFAASMGMQTTGIGGTQENYYMLREGEKHWSDPWHWAQAAIVAACRTHGVLPVDGPFGDFSDDEGYIAQAKRSATLGMVGKWAIHPKQIALANEVFTPSEEAVAEAREILAAMEQAKANGEGATVYKGRLVDIASIKQAEVIVAQSELIAKG

SEQ ID NO: 29 - protein, 319 aa, Roseovarius nubinhibens
MSFRLQPPAPARPNRCQLFGPASNKKLFEKMAASDADVINIDLEDSVAPTDKDAARANAIEAINGLNWGNKTLSVRINGLDTGWWYRDVVDLLEQCGDRLDQIMIPKVGCAADIYAVDALVTSIERARGKDKRISFEVIIESAAGIAHVEEIAAASPRLEAMSLGAADFAASMGMATTGIGGTQENYYMIREGQSYWPDPWHWATAAIVAACRTHGVLPVDGPFGDFSDDEGFRAQARRAATLGMVGKWAIHPKQIALANEVFTPSDEAVAEAREILAAMEEAKAKGEGATVYKGRLVDIASIKQAEVIVKQSEMIAGS

SEQ ID NO: 30 - protein, 348 aa, unidentified
MSKPARKFLEPLAIGAPEPYRELPVALERMIHFFPPHIEKMRAKVPDMAKQVDVLLGNLEDAIPADAKEAARAGFIEVAKNADLGDTGLWTRVNCLNSPWFLDDVIQVVDAAGDKLDVVMLPKVEGPWDIHYLDQLLAQLEAKNSVKRPIMIHAILETALGVENIGAIAAASPRMHGMSLGPADLAASRGMKTTRVGGGHPFYGVMEDPRDDDAPRAFYQQDLWHYTVAKMVDACQANGIKAFYGPFGDFSDNEACEAQFRNAFLLGCAGAWSLHPKQIDIAKNVFSPDPEEVIFALKILDAMPDGTGAVMIDGKMQDDATWKQAKVIADLAKQVAAKDPEMAKVYGL

SEQ ID NO: 31 - protein, 350 aa, Saccharomyces cerevisiae
MSKKPIVLKLGKDAFGDQAWGELEKIADVITIPESTTREQFLREVKDPQNKLSQVQVITRTARSVKNTGRFDEELALALPSSVVAVCHTGAGYDQIDVEPFKKRHIQVANVPDLVSNATADTHVFLLLGALRNFGIGNRRLIEGNWPEAGPACGSPFGYDPEGKTVGILGLGRIGRCILERLKPFGFENFIYHNRHQLPSEEEHGCEYVGFEEFLKRSDIVSVNVPLNHNTHHLINAETIEKMKDGVVIVNTARGAVIDEQAMTDALRSGKIRSAGLDVFEYEPKISKELLSMSQVLGLPHMGTHSVETRKKMEELVVENAKNVILTGKVLTIVPELQNEDWPNESKPLV

SEQ ID NO: 32 - protein, 300 aa, Sulfolobus solfataricus; identical to SEQ ID NO: 18

SEQ ID NO: 33 - protein, 312 aa, Escherichia coli
MDIIFYHPTFDTQWWIEALRKAIPQARVRAWKSGDNDSADYALVWHPPVEMLAGRDLKAVFALGAGVDSILSKLQAHPEMLNPSVPLFRLEDTGMGEQMQEYAVSQVLHWFRRFDDYRIQQNSSHWQPLPEYHREDFTIGILGAGVLGSKVAQSLQTWRFPLRCWSRTRKSWPGVQSFAGREELSAFLSQCRVLINLLPNTPETVGIINQQLLEKLPDGAYLLNLARGVHVVEDDLLAALDSGKVKGAMLDVFNREPLPPESPLWQHPRVTITPHVAAITRPAEAVEYISRTIAQLEKGERVCGQVDRARGY

SEQ ID NO: 34 - protein, 323 aa, Klebsiella pneumoniae; identical to SEQ ID NO: 17

SEQ ID NO: 35 - protein, 1511 aa, Saccharomyces cerevisiae
MSSTDEHIEKDISSRSNHDDDYANSVQSYAASEGQVDNEDLAATSQLSRHLSNILSNEEGIERLESMARVISHKTKKEMDSFEINDLDFDLRSLLHYLRSRQLEQGIEPGDSGIAFKNLTAVGVDASAAYGPSVEEMFRNIASIPAHLISKFTKKSDVPLRNIIQNCTGVVESGEMLFVVGRPGAGCSTFLKCLSGETSELVDVQGEFSYDGLDQSEMMSKYKGYVIYCPELDFHFPKITVKETIDFALKCKTPRVRIDKMTRKQYVDNIRDMWCTVFGLRHTYATKVGNDFVRGVSGGERKRVSLVEAQAMNASIYSWDNATRGLDASTALEFAQAIRTATNMVNNSAIVAIYQAGENIYELFDKTTVLYNGRQIYFGPADKAVGYFQRMGWVKPNRMTSAEFLTSVTVDFENRTLDIKPGYEDKVPKSSSEFEEYWLNSEDYQELLRTYDDYQSRHPVNETRDRLDVAKKQRLQQGQRENSQYVVNYWTQVYYCMIRGFQRVKGDSTYTKVYLSSFLIKALIIGSMFHKIDDKSQSTTAGAYSRGGMLFYVLLFASVTSLAEIGNSFSSRPVIVKHKSYSMYHLSAESLQEIITEFPTKFVAIVILCLITYWIPFMKYEAGAFFQYILYLLTVQQCTSFIFKFVATMSKSGVDAHAVGGLWVLMLCVYAGFVLPIGEMHHWIRWLHFINPLTYAFESLVSTEFHHREMLCSALVPSGPGYEGISIANQVCDAAGAVKGNLYVSGDSYILHQYHFAYKHAWRNWGVNIVWTFGYIVFNVILSEYLKPVEGGGDLLLYKRGHMPELGTENADARTASREEMMEALNGPNVDLEKVIAEKDVFTWNHLDYTIPYDGATRKLLSDVFGYVKPGKMTALMGESGAGKTTLLNVLAQRINMGVITGDMLVNAKPLPASFNRSCGYVAQADNHMAELSVRESLRFAAELRQQSSVPLEEKYEYVEKIITLLGMQNYAEALVGKTGRGLNVEQRKKLSIGVELVAKPSLLLFLDEPTSGLDSQSAWSIVQFMRALADSGQSILCTIHQPSATLFEQFDRLLLLKKGGKMVYFGDIGPNSETLLKYFERQSGMKCGVSENPAEYILNCIGAGATASVNSDWHDLWLASPECAAARAEVEELHRTLPGRAVNDDPELATRFAASYMTQIKCVLRRTALQFWRSPVYIRAKFFECVACALFVGLSYVGVNHSVGGAIEAFSSIFMLLLIALAMINQLHVFAYDSRELYEVREAASNTFHWSVLLLCHAAVENFWSTLCQFMCFICYYWPAQFSGRASHAGFFFFFYVLIFPLYFVTYGLWILYMSPDVPSASMINSNLFAAMLLFCGILQPREKMPAFWRRLMYNVSPFTYVVQALVTPLVHNKKVVCNPHEYNIMDPPSGKTCGEFLSTYMDNNTGYLVNPTATENCQYCPYTVQDQVVAKYNVKWDHRWRNFGFMWAYICFNIAAMLICYYVVRVKVWSLKSVLNFKKWFNGPRKERHEKDTNIFQTVPGDENKITKK

SEQ ID NO: 36 - protein, 944 aa, Saccharomyces cerevisiae
MDTQIAITGVAVGKEINNDNSKTDQKVSLPKADVPCIDKATQTIIEGCSKDDPRLSYPTKLETTEKGKTKRNSFACVCCHSLKQKCEPSDVNDIYRKPCRRCLKHKKLCKFDLSKRTRKRKPRSRSPTPFESPMVNVSTKSKGPTDSEESSLKDGTSYLASFPSDPNAKQFPNSRTVLPGLQQSLSDLWSTLSQPPSYGAREAETTSTGEITTNNHTKSNGSVPTNPAVLASNDEHTNISDAPVIYSTYNSPVPISSAPTSINSEALFKHRPKIVGDEETQNVKVKRQKKSYSRHMTRSFRKQLQSLIISQKGKIRDISMKLDTWSKQWNDLVEKSMFLPTIADPVSVGIISHEEATLRLHLYKTEISYLSKLPFIKVEENVSVDELRKKKPILFSVIMSCVSIVLTPKQTTRGTIMKLDSFVLNLITNQIFKANNKSIEIIESLSTLCLWYNFFEWSSKTRYHIFNYICCCLTRDLGPTYVNRSFGMFSDEDPKRFKSPLELYSNGASLTLLVYISALNISIFLRQSIQARWSHVTEKACEDLVKETKKSRHYDNDKLLLDSADDPILVQFAKMNHVLENIHTHLHERDLNDDEFDDPIFTKKYLNKLMEKYHKQLQEIFTKLDRNRPRVIAFYYSVEAYLYQYKLAVFIGEMSHTINEKVELPREIMDDFVKCYHCCKSALEEFSKLEPILITSLPLFHTSRIIYTVGMLLLKLRYSVVAIPSFHDLMPLTDDAIALVIGVNNLLEKTSELYPFNNSLYKFRYVIALFCQTYANKVIDVADRYNAEREKLKEKQVIDEVSNGHDGTKPINAYVTESQKMPTEEDPIIDNNTNQNITAVPDEMLPVYSRVRDDTAAMNLNINSTSYMNESPHEHRESMTGTTLLPPPFISNDVTNSADSTNIKPSPSSSVDNLNDYLTDINSLAWGVNSLNDEFWTDLFMNDI

SEQ ID NO: 37 - protein, 438 aa, unidentified
MGELKEILKQRYHELLDWNVKAPHVPLSQRLKHFTWSWFACTMATGGVGLIIGSFPFRFYGLNTIGKIVYILQIFLFSLFGSCMLFRFIKYPSTIKDSWNHHLEKLFIATCLLSISTFIDMLAIYAYPDTGEWMVWVIRILYYIYVAVSFIYCVMAFFTIFNNHVYTIETASPAWILPIFPPMICGVIAGAVNSTQPAHQLKNMVIFGILFQGLGFWVYLLLFAVNVLRFFTVGLAKPQDRPGMFMFVGPPAFSGLALINIARGAMGSRPYIFVGANSSEYLGFVSTFMAIFIWGLAAWCYCLAMVSFLAGFFTRAPLKFACGWFAFIFPNVGFVNCTIEIGKMIDSKAFQMFGHIIGVILCIQWILLMYLMVRAFLVNDLCYPGKDEDAHPPPKPNTGVLNPTFPPEKAPASLEKVDTHVTSTGGESDPPSSEHESV

SEQ ID NO: 38 - protein, 1517 aa, unidentified
MSNSSSSEGKTNEDGRNSVHSSDSFAQSVASFHLDDNESQNVTAQLSQQITNVLSNSNGAERIESLARVISTKTKKQMESFEVNQLDFDLKALLNYLRSSQLEQGIEPGDSGIAFHDLTAVGIDASAAFGPSVEEMVRSWIHFPVRLWKKICRQKSETPLRNIIQHCTGVVESGEMLFVVGRPGAGCSTLLKCLSGETGELVEVTGDISYDGLSQEEMMQKFKGYVIYCPELDFHFPKITVKETIDFALKCKTPRSRIDHLTRAQYVDNMRDLWCTVFGLTHTYATNVGNDVVRGVSGGERKRVSLVEALAMNASIYSWDNATRGLDASTALEFAQAIRTATNMMNNSAIVAIYQAGENIYQLFDKTTVLYNGKQVYFGPADEAVGYFERMGYIKPNRMTSAEFLTSATVDFENRTLEVREGYEEKIPKSSTEMEAYWHNSPEYAKATELFNEYCQSHPEEETRQRLETAKKQRLQKGQREKSQFVVTFWAQVWYCMIRGFQRVKGDSTYTKVYLSSFLTKGLIVGSMFHKIDPKSQSTTEGAYSRGGLLFYVLLFAALTSLAEISNSFQNRAIIVKQKTYSMYHTSAESLQEIFTEIPTKFVAILTLSLVSYWIPVLKYDAGSFFQYLLYLFTTQQCTSFIFKLVATLTKDGGTAHAIGGLWVLMLTVYAGFVLPIGNMHHWIRWFHYLNPLTYAYESLMSTEFHGRKMLCSRLLPSGPGYENVSIAHKICDAAGAVAGQLYVSGDAYVLKKYHFRYKHAWRDWGINIVWTFGYIVMNVVMSEYLKPLEGGGDLLLYKRGHMPELGSESVDSKVASREEMMESLNGPGVDLEKVIASKDVFTWNHLNYTIPYDGATRQLLSDVFGYVKPGKMTALMGESGAGKTTLLNVLAQRINVGVITGDMLVNAKPLPPSFNRSCGYVAQADNHMGELSVRESLRFAAELRQPKSVPLQEKYDYVEKIISLLGMEKYAEAIIGKTGRGLNVEQRKKLSIGVELVAKPSLLLFLDEPTSGLDSQSAWSIVQFMRALADSGQSILCTIHQPSATLFEQFDRLLLLKKGGKMVYFGDIGENSSTLLNYFERQSGVKCGKSENPAEYMLNCIGAGATASADADWHDLWLQSPECAAAREEVEELHRTLASRPVTDDKELAGRYAASYLTQMKCVFRRTNIQFWRSPVYIRAKFLECVLCALFVGLSYVGVDHSIAGASQSFSSIFMMLLIALAMVNQLHVFALDSRELYEVREAASNTFHWSVLLLNHTFVEIIWSTLCEFICWICYYWPAQYSGRASHAGYFFLIYVIMFPAYFVSYGCWVFYMSPDVPSASMINSNLFAGMLLFCGILQPKDKMPGFWKRFMYNVSPFTYVVQSLVTPLVQGKKVRCTKNEFAVVNPPEGQTCSQYFARFIKDNTGYLKNPNDTESCHYCPYSYQQEVVEQYNVRWVYRWRNFGFLWAYIGFNFFAMLACYWVLRVKNYSITSIFGVFKIGNWKKAIHHDSRHEKDHTIFQEKPGDAANVQKTKA

SEQ ID NO: 39 - protein, 575 aa, unidentified
MTDKISLGTYLFEKLKEAGSYSIFGVPGDFNLALLDHVKEVEGIRWVGNANELNAGYEADGYARINGFASLITTFGVGELSAVNAIAGSYAEHVPLIHIVGMPSLSAMKNNLLLHHTLGDTRFDNFTEMSKKISAKVEIVYDLESAPKLINNLIETAYHTKRPVYLGLPSNFADELVPAALVKENKLHLEEPLNNPVAEEEFIHNVVEMVKKAEKPIILVDACAARHNISKEVRELAKLTKFPVFTTPMGKSTVDEDDEEFFGLYLGSLSAPDVKDIVGPTDCILSLGGLPSDFNTGSFSYGYTTKNVVEFHSNYCKFKSATYENLMMKGAVQRLISELKNIKYSNVSTLSPPKSKFAYESAKVAPEGIITQDYLWKRLSYFLKPRDIIVTETGTSSFGVLATHLPRDSKSISQVLWGSIGFSLPAAVGAAFAAEDAHKQTGEQERRTVLFIGDGSLQLTVQSISDAARWNIKPYIFILNNRGYTIEKLIHGRHEDYNQIQPWDHQLLLKLFADKTQYENHVVKSAKDLDALMKDEAFNKEDKIRVIELFLDEFDAPEILVAQAKLSDEINSKAA

SEQ ID NO: 40 - protein, 608 aa, unidentified
MLQTANSEVPNASQITIDAASGLPADRVLPNITNTEITISEYIFYRILQLGVRSVFGVPGDFNLRFLEHIYDVHGLNWIGCCNELNAAYAADAYAKASKKMGVLLTTYGVGELSALNGVAGAYTEFAPVLHLVGTSALKFKRNPRTLNLHHLAGDKKTFKKSDHYKYERIASEFSVDSASIEDDPIEACEMIDRVIYSTWRESRPGYIFLPCDLSEMKVDAQRLASPIELTYRFNSPVSRVEGVADQILQLIYQNKNVSIIVDGFIRKFRMESEFYDIMEKFGDKVNIFSTMYGKGLIGEEHPRFVGTYFGKYEKAVGNLLEASDLIIHFGNFDHELNMGGFTFNIPQEKYIDLSAQYVDITGNLDESITMMEVLPVLASKLDSSRVNVADKFEKFDKYYETPDYQREASLQETDIMQSLNENLTGDDILIVETCSFLFAVPDLKVKQHTNIILQAYWASIGYALPATLGASLAIRDFNLSGKVYTIEGDGSAQMSLQELSSMLRYNIDATMILLNNSGYTIERVIVGPHSSYNDINTNWQWTDLLRAFGDVANEKSVSYTIKEREQLLNILSDPSFKHNGKFRLLECVLPMFDVPKKLGQFTGKIPA

SEQ ID NO: 41 - protein, 584 aa, unidentified
MAPVSLETCTLEFSCKLPLSEYIFRRIASLGIHNIFGVPGDYNLSFLEHLYSVPELSWVGCCNELNSAYATDGYSRTIGHDKFGVLLTTQGVGELSAANAIAGSFAEHVPILHIVGTTPYSLKHKGSHHHHLINGVSTREPTNHYAYEEMSKNISCKILSLSDDLTNAANEIDDLFRTILMLKKPGYLYIPCDLVNVEIDASNLQSVPANKLRERVPSTDSQTIAKITSTIVDKLLSSSNPVVLCDILTDRYGMTAYAQDLVDSLKVPCCNSFMGKALLNESKEHYIGDFNGEESNKMVHSYISNTDCFLHIGDYYNEINSGHWSLYNGINKESIVILNPEYVKIGSQTYQNVSFEDILPAILSSIKANPNLPCFHIPKIMSTIEQIPSNTPISQTLMLEKLQSFLKPNDVLVTETCSLMFGLPDIRMPENSKVIGQHFYLSIGMALPCSFGVSVALNELKKDSRLILIEGDGSAQMTVQELSNFNRENVVKPLIILLNNSGYTVERVIKGPKREYNDIRPDWKWTQLLQTFGMDDAKSMKVTTPEELDDALDEYGNNLSTPRLLEVVLDKLDVPWRFNKMVGN

SEQ ID NO: 42 - protein, 407 aa, unidentified
MLRLFSRRTPSVRALPKFTRSLATASPEAGAQEVSNLHDIVEIELPEYSFEGYKLDVPELNYSTEKGTLLQMYKDMVIIRRMEMAADALYKAKKIRGFCHLSVGQEAIAVGIENAITKQDDIITSYRCHGTTYMRGASVQEVLAELMGRRSGVSYGKGGSMHMYTKGFYGGNGIVGAQVPLGTGLAFAHHYRDQKNMTWTMYGDGAANQGQVFESFNMAKLWNLPCVFTCENNKYGMGTSASRSSAMTEYYKRGQYIPGLKVNGMDILAVYQAAKFAKEWTSNDNGPLVIEFETYRYGGHSMSDPGTTYRTREEVQNMRSKKDPIAGLKAHLLEFNIATEEEIKAFDKSARKYVDEQVKLADASPPPEAKMSILFEDVYVPGSEIPVLRGRIRDDSWSFEKGGFAYK

SEQ ID NO: 43 - protein, 388 aa, unidentified
MVSPAERLSTIASTIKPNRKDSTSLQPEDYPEHPFKVTVVGSGNWGCTIAKVIAENTVERPRQFQRDVNMWVYEELIEGEKLTEIINTKHENVKYLPGIKLPVNVVAVPDIVEACAGSDLIVFNIPHQFLPRILSQLKGKVNPKARAISCLKGLDVNPNGCKLLSTVITEELGIYCGALSGANLAPEVAQCKWSETTVAYTIPDDFRGKGKDIDHQILKSLFHRPYFHVRVISDVAGISIAGALKNVVAMAAGFVEGLGWGDNAKAAVMRIGLVETIQFAKTFFDGCHAATFTHESAGVADLITTCAGGRNVRVGRYMAQHSVSATEAEEKLLNGQSCQGIHTTREVYEFLSNMGRTDEFPLFTTTYRIIYENFPIEKLPECLEPVED

SEQ ID NO: 44 - protein, 255 aa, unidentified
MSAPSTATQNLKNLKAAAPQSVVGADHAPHVNPNYPFTLSKNSSLGDYLQANRLNTEKLNKSHPEILPLSGQGQQPHTLWIGCSDSRINECQALGCLPGEIFTLRNIANVISSTDISSMSALQFAIEVLKVKKIIVCGHTDCGGVWASLSNKKAGGVLDTWLSAVRHVRAQHLDELREIEDINDKCRRLVELNILNSINTIKKNPSFVEYHARGDIELYALIYDVKTGYLNEIPLEDDTYAEVFHLHDNDGEPVH

SEQ ID NO: 45 - protein, 862 aa, unidentified
MVMKTIACVACKRRKVRCDSSKQVPCSNCVRYGKHCIASDDRRSLRPTYQELETLRERVKQLERHVQELNSDVLIQREEVMQEASPVYPKPRSFQSLYRHKVNIYGPPSIFDEDGEDGEREGENLDDEEGGDDAENNMKKRKMDMYKNERLGYLGENVDTISDCSSERSISKRNNLSKIVLVSPVNKLNKDPKVIQFVKTFFQWFYPDLHMFIPRETFLVEFYHPRREQVYCNIALVYAICAIAGGGDGYYEVARKQMEKPSSAVIGIQGNILLGLYELRKGDRRKAWLSVGLGLRIGLESGFHLKLERANKLMVLFRSRIYWGCFIIDHLMSFLLGRPPMLRIEESSIEESEKVPDLEWIKEFNFAGKGILDVSATLRAIVSLIIIVDEWILGRCEDVELINGHLQRWRNELSEEMQWDEGKLKSKAYHPPDVVHIMYYYMVFIGINRLYGEGEFLDNAINEGVLVIQTHIEAHGIMRCTLLMHCLARTILKVRPGHSLCVGILEERVPNTGADHDAGVNALSSIGVGGGGGDDDAQNAVYNVTQNGNYNMQTGDTHTNHFLDMELDLSDTADSDMNMILDIERSNSNLTIKIPFKRFATELIPRAMSAPSTATQNLKNLKAAAPQSVVGADHAPHVNPNYPFTLSKNSSLGDYLQANRLNTEKLNKSHPEILPLSGQGQQPHTLWIGCSDSRINECQALGCLPGEIFTLRNIANVISSTDISSMSALQFAIEVLKVKKIIVCGHTDCGGVWASLSNKKAGGVLDTWLSAVRHVRAQHLDELREIEDINDKCRRLVELNILNSINTIKKNPSFVEYHARGDIELYALIYDVKTGYLNEIPLEDDTYAEVFHLHDNDGEPVH

SEQ ID NO: 46 - protein, 260 aa, Homo sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 47 - protein, 330 aa, Flaveria bidentis
MSAASAFAMNAPSFVNASSLKKASTSARSGVLSARFTCNSSSSSSSSATPPSLIRNEPVFAAPAPIITPNWTEDGNESYEEAIDALKKTLIEKGELEPVAATRIDQITAQAAAPDTKAPFDPVERIKSGFVKFKTEKFVTNPALYDELAKGQSPKFMVFACSDSRVCPSHVLDFQPGEAFVVRNVANMVPPFDKTKYSGVGAAVEYAVLHLKVQEIFVIGHSRCGGIKGLMTFPDEGPHSTDFIEDWVKVCLPAKSKVVAEHNGTHLDDQCVLCEKEAVNVSLGNLLTYPFVRDGLRNKTLALKGGHYDFVNGTFELWALDFGLSSPTSV

SEQ ID NO: 48 - protein, 221 aa, Saccharomyces cerevisiae
MSATESSSIFTLSHNSNLQDILAANAKWASQMNNIQPTLFPDHNAKGQSPHTLFIGCSDSRYNENCLGVLPGEVFTWKNVANICHSEDLTLKATLEFAIICLKVNKVIICGHTDCGGIKTCLTNQREALPKVNCSHLYKYLDDIDTMYHEESQNLIHLKTQREKSHYLSHCNVKRQFNRIIENPTVQTAVQNGELQVYGLLYNVEDGLLQTVSTYTKVTPK

SEQ ID NO: 49 - protein, 281 aa, Candida albicans
MGRENILKYQLEHDHESDLVTEKDQSLLLDNNNNLNGMNNTIKTHPVRVSSGNHNNFPFTLSSESTLQDFLNNNKFFVDSIKHNHGNQIFDLNGQGQSPHTLWIGCSDSRAGDQCLATLPGEIFVHRNIANIVNANDISSQGVIQFAIDVLKVKKIIVCGHTDCGGIWASLSKKKIGGVLDLWLNPVRHIRAANLKLLEEYNQDPKLKAKKLAELNVISSVTALKRHPSASVALKKNEIEVWGMLYDVATGYLSQVEIPQDEFEDLFHVHDEHDEEEYNPH

SEQ ID NO: 50 - protein, 192 aa, Porphyromonas gingivalis
MAQRENSDYLTTKMALIQSVRGFTPIIGEDTFLAENATIVGDVVMGKGCSVWFNAVLRGDVNSIRIGDNVNIQDGSILHTLYQKSTIEIGDNVSVGHNVVIHGAKICDYALIGMGAVVLDHVVVGEGAIVAAGSVVLTGTQIEPNSIYAGAPARFIKKVDPEQSREMNFRIAHNYRMYASWFKDESSEIDNP

SEQ ID NO: 51 - protein, 207 aa, Mycobacterium tuberculosis
MPNTNPVAAWKALKEGNERFVAGRPQHPSQSVDHRAGLAAGQKPTAVIFGCADSRVAAEIIFDQGLGDMFVVRTAGHVIDSAVLGSIEYAVTVLNVPLIVVLGHDSCGAVNAALAAINDGTLPGGYVRDVVERVAPSVLLGRRDGLSRVDEFEQRHVHETVAILMARSSAISERIAGGSLAIVGVTYQLDDGRAVLRDHIGNIGEEV

SEQ ID NO: 52 - protein, 295 aa, Roseobacter denitrificans
MSILLDSDTKVIVQGITGKMARFHTKDMLEYGTNVVGGVVPGKGGETVEGVPVFDTVEEAVAQTGAEASLVFVPPPFAADSIMEAADAGIQYCVCITDGIPAQDMIRVKRYMYRYPKERRMVLTGPNCAGTISPGKALLGIMPGHIYLQGHVGIVGRSGTLGYEAAAQLKERGIGVSTSVGIGGDPINGSSFKDILQRFEEDEDTHVIAMIGEIGGPQEAEAAEYIRAHVTKPVVAYVAGLTAPKGRTMGHAGAIISAFGESASEKVEILSAAGVTVAENPAVIGETIAKVMEAA

SEQ ID NO: 53 - protein, 1193 aa, unidentified
MAAPFRQPEEAVDDTEFIDDHHEHLRDTVHHRLRANSSIMHFQKILVANRGEIPIRIFRTAHELSLQTVAIYSHEDRLSMHRQKADEAYMIGHRGQYTPVGAYLAGDEIIKIALEHGVQLIHPGYGFLSENADFARKVENAGIVFVGPTPDTIDSLGDKVSARRLAIKCEVPVVPGTEGPVERYEEVKAFTDTYGFPIIIKAAFGGGGRGMRVVRDQAELRDSFERATSEARSAFGNGTVFVERFLDKPKHIEVQLLGDSHGNVVHLFERDCSVQRRHQKVVEVAPAKDLPADVRDRILADAVKLAKSVNYRNAGTAEFLVDQQNRHYFIEINPRIQVEHTITEEITGIDIVAAQIQIAAGASLEQLGLTQDRISARGFAIQCRITTEDPAKGFSPDTGKIEVYRSAGGNGVRLDGGNGFAGAIITPHYDSMLVKCTCRGSTYEIARRKVVRALVEFRIRGVKTNIPFLTSLLSHPTFVDGNCWTTFIDDTPELFSLVGSQNRAQKLLAYLGDVAVNGSSIKGQIGEPKLKGDVIKPKLFDAEGKPLDVSAPCTKGWKQILDREGPAAFAKAVRANKGCLIMDTTWRDAHQSLLATRVRTIDLLNIAHETSYAYSNAYSLECWGGATFDVAMRFLYEDPWDRLRKMRKAVPNIPFQMLLRGANGVAYSSLPDNAIYHFCKQAKKCGVDIFRVFDALNDVDQLEVGIKAVHAAEGVVEATMCYSGDMLNPHKKYNLEYYMALVDKIVAMKPHILGIKDMAGVLKPQAARLLVGSIRQRYPDLPIHVHTHDSAGTGVASMIACAQAGADAVDAATDSMSGMTSQPSIGAILASLEGTEQDPGLNLAHVRAIDSYWAQLRLLYSPFEAGLTGPDPEVYEHEIPGGQLTNLIFQASQLGLGQQWAETKKAYEAANDLLGDIVKVTPTSKVVGDLAQFMVSNKLTPEDVVERAGELDFPGSVLEFLEGLMGQPFGGFPEPLRSRALRDRRKLEKRPGLYLEPLDLAKIKSQIREKFGAATEYDVASYAMYPKVFEDYKKFVQKFGDLSVLPTRYFLAKPEIGEEFHVELEKGKVLILKLLAIGPLSEQTGQREVFYEVNGEVRQVAVDDNKASVDNTSRPKADVGDSSQVGAPMSGVVVEIRVHDGLEVKKGDPLAVLSAMKMEMVISAPHSGKVSSLLVKEGDSVDGQDLVCKIVKA

SEQ ID NO: 54 - protein, 549 aa, Saccharomyces cerevisiae
MSPSKMNATVGSTSEVEQKIRQELALSDEVTTIRRNAPAAVLYEDGLKENKTVISSSGALIAYSGVKTGRSPKDKRIVEEPTSKDEIWWGPVNKPCSERTWSINRERAADYLRTRDHIYIVDAFAGWDPKYRIKVRVVCARAYHALFMTNMLIRPTEEELAHFGEPDFTVWNAGQFPANLHTQDMSSKSTIEINFKAMEMIILGTEYAGEMKKGIFTVMFYLMPVHHNVLTLHSSANQGIQNGDVTLFFGLSGTGKTTLSADPHRLLIGDDEHCWSDHGVFNIEGGCYAKCINLSAEKEPEIFDAIKFGSVLENVIYDEKSHVVDYDDSSITENTRCAYPIDYIPSAKIPCLADSHPKNIILLTCDASGVLPPVSKLTPEQVMYHFISGYTSKMAGTEQGVTEPEPTFSSCFGQPFLALHPIRYATMLATKMSQHKANAYLINTGWTGSSYVSGGKRCPLKYTRAILDSIHDGSLANETYETLPIFNLQVPTKVNGVPAELLNPAKNWSQGESKYRGAVTNLANLFVQNFKIYQDRATPDVLAAGPQFE

SEQ ID NO: 55 - protein, 560 aa, unidentified
MAPTAVDIHNEYKQNVSNEQEIPFNKTERKSSIASKLGLNPDAKIHYNSAVPILYEDGLKEKGTTISSSGALIAFSGSKTGRSPKDKRIVDEETSTDNIWWGPVNKKVDENTWNISKSRAIDYLRTREKVYIIDAFAGWDPRYRIKVRIVCARAYHALFMKNMLIRPTTEELKNFGEPDFTIWNAGQFPANVYTKGMTSSTSVEINFKSMEMVILGTEYAGEMKKGIFTVMFYLMPIRHKVLTLHSSANQGKKDGDVTLFFGLSGTGKTTLSADPHRELIGDDEHCWSDHGVFNIEGGCYAKCLDLSAEREPEIFNAIRFGSVLENVVYDPVDRTVDYSAANVTENTRCAYPIDFIPSAKIPCLADSHPKNIVLLTCDARGVLPPVSKLTNAQVMYHFISGYTSKMAGTEVGVTEPEATFSACFGQPFLVLHPMKYAQQLSDKMAEHSSTAWLLNTGWTGQSYVKGGKRCPLKYTRAILDAIHSGELAKQEFETYPTFGLQVPKTCPGVPESVLNPSKHWATGEADFKAEVTNLAKLFAENFEKYSAECTAEVVAAGPAL

SEQ ID NO: 56 - protein, 399 aa, Roseobacter denitrificans
MDIHEYQAKEVLANFGVAVPDGALAYSPEQASYRARELGGERWVVKAQIHAGGRGKAGGVKVCESDVEIQKASEEMFGKKMITHQTGPEGKGIYRVYVEAAVPIDREIYLGFVLDRTTQRVMIVASAEGGMEIEEISEKRPESIVRAIVEPAVGLREFQCRQIAFKLGVDAGLTQQMVRTLQGCYRAFTELDATMVEINPLVITADNRVIALDAKMTFDDNALFRHPQISELRDKSQEDPRESRAADRGLSYVGLDGNIGCIVNGAGLAMATMDTIKLAGGEPANFLDIGGGATPERVAKAFRLVLSDKSVQAILVNIFAGINRCDWVAEGVVQALREVQVDVPVIVRLAGTNVEEGQKILAQSGLPIIRATTLMEAAERAVGAWQNDLSQGTRMRAVS

SEQ ID NO: 57 - protein, 878 aa, synthetic construct
MNEQYSALRSNVSMLGKLLGDTIKDALGEDILDRVETIRKLSKSSRAGNEANRQELLTTLQNLSNDELLPVARAFSQFLNLTNTAEQYHSISPHGEAASNPEAIARLFRKLKDQNLSEADIKKAVESLSIELVLTAHPTEITRRTLIHKLVEVNTCLSQLDHDDLADYERNQIMRRLRQLIAQSWHTDEIRKQRPTPVDEAKWGFAVVENSLWEGVPAFLREFNEQLEESLGYKLPVEAVPVRFTSWMGGDRDGNPNVTAEITRHVLLLSRWKAADLFLKDIQVLVSELSMVECTPELRALAGEEGALEPYRELLKNLRTQLMATLAYLEARLKGERLPKPAGLLTQNEQLWEPLYACYQSLQACGMGIIANGQLLDTLRRVKCFGVPLVRIDIRQESTRHTEALAELTRYLGLGDYESWSEADKQAFLIRELNSKRPLLPRNWEPSAETQEVLDTCKVIAEAPKGSIAAYVISMAKTPSDVLAVHLLLKEAGCPFALPVAPLFETLDDLNNAEDVMTQLLNIDWYRGFIQGKQMVMIGYSDSAKDAGVMAASWAQYRAQDALIKTCEKAGIELTLFHGRGGSIGRGGAPAHAALLSQPPGSLKGGLRVTEQGEMIRFKLGLPEVTVSSLSLYASAILEANLLPPPEPKDEWRDIMDELSDISCDLYRGYVRENKDFVPYFRAATPEQELGKLPLGSRPAKRRPTGGVESLRAIPWIFAWTQNRLMLPAWLGAGAALQKVIEDGKQDELEAMCRDWPFFSTRIGMLEMVFAKADLWLAEYYDQRLVDKALWPLGEELRDQLEEDIKVVLAIANDSHLMADLPWIAESIALRNVYTDPLNVLQAELLHRSRQQEEPDPRVEQALMVTIAGIAAGMRNTG

SEQ ID NO: 58 - protein, 883 aa, Escherichia coli
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 59 - protein, 606 aa, Mycobacterium tuberculosis
MTSATIPGLDTAPTNHQGLLSWVEEVAELTQPDRVVFTDGSEEEFQRLCDQLVEAGTFIRLNPEKHKNSYLALSDPSDVARVESRTYICSAKEIDAGPTNNWMDPGEMRSIMKDLYRGCMRGRTMYVVPFCMGPLGAEDPKLGVEITDSEYVVVSMRTMTRMGKAALEKMGDDGFFVKALHSVGAPLEPGQKDVAWPCSETKYITHFPETREIWSYGSGYGGNALLGKKCYSLRIASAMAHDEGWLAEHMLILKLISPENKAYYFAAAFPSACGKTNLAMLQPTIPGWRAETLGDDIAWMRFGKDGRLYAVNPEFGFFGVAPGTNWKSNPNAMRTIAAGNTVFTNVALTDDGDVWWEGLEGDPQHLIDWKGNDWYFRETETNAAHPNSRYCTPMSQCPILAPEWDDPQGVPISGILFGGRRKTTVPLVTEARDWQHGVFIGATLGSEQTAAAEGKVGNVRRDPMAMLPFLGYNVGDYFQHWINLGKHADESKLPKVFFVNWFRRGDDGRFLWPGFGENSRVLKWIVDRIEHKAGGATTPIGTVPAVEDLDLDGLDVDAADVAAALAVDADEWRQELPLIEEWLQFVGEKLPTGVKDEFDALKERLG

SEQ ID NO: 60 - protein, 919 aa, Corynebacterium glutamicum
MTDFLRDDIRFLGQILGEVIAEQEGQEVYELVEQARLTSFDIAKGNAEMDSLVQVFDGITPAKATPIARAFSHFALLANLAEDLYDEELREQALDAGDTPPDSTLDATWLKLNEGNVGAEAVADVLRNAEVAPVLTAHPTETRRRTVFDAQKWITTHMRERHALQSAEPTARTQSKLDEIEKNIRRRITILWQTALIRVARPRIEDEIEVGLRYYKLSLLEEIPRINRDVAVELRERFGEGVPLKPVVKPGSWIGGDHDGNPYVTAETVEYSTHRAAETVLKYYARQLHSLEHELSLSDRMNKVTPQLLALADAGHNDVPSRVDEPYRRAVHGVRGRILATTAELIGEDAVEGVWFKVFTPYASPEEFLNDALTIDHSLRESKDVLIADDRLSVLISAIESFGFNLYALDLRQNSESYEDVLTELFERAQVTANYRELSEAEKLEVLLKELRSPRPLIPHGSDEYSEVTDRELGIFRTASEAVKKFGPRMVPHCIISMASSVTDVLEPMVLLKEFGLIAANGDNPRGTVDVIPLFETIEDLQAGAGILDELWKIDLYRNYLLQRDNVQEVMLGYSDSNKDGGYFSANWALYDAELQLVELCRSAGVKLRLFHGRGGTVGRGGGPSYDAILAQPRGAVQGSVRITEQGEIISAKYGNPETARRNLEALVSATLEASLLDVSELTDHQRAYDIMSEISELSLKKYASLVHEDQGFIDYFTQSTPLQEIGSLNIGSRPSSRKQTSSVEDLRAIPWVLSWSQSRVMLPGWFGVGTALEQWIGEGEQATQRIAELQTLNESWPFFTSVLDNMAQVMSKAELRLAKLYADLIPDTEVAERVYSVIREEYFLTKKMFCVITGSDDLLDDNPLLARSVQRRYPYLLPLNVIQVEMMRRYRKGDQSEQVSRNIQLTMNGLSTALRNSG

SEQ ID NO: 61 - protein, 432 aa, Corynebacterium glutamicum
MSNVGKPRTAQEIQQDWDTNPRWNGITRDYTADQVADLQGSVIEEHTLARRGSEILWDAVTQEGDGYINALGALTGNQAVQQVRAGLKAVYLSGWQVAGDANLSGHTYPDQSLYPANSVPSVVRRINNALLRSDEIARTEGDTSVDNWVVPIVADGEAGFGGALNVYELQKAMIAAGAAGTHWEDQLASEKKCGHLGGKVLIPTQQHIRTLNSARLAADVANTPTVVIARTDAEAATLITSDVDERDQPFITGERTAEGYYHVKNGLEPCIARAKSYAPYADMIWMETGTPDLELAKKFAEGVRSEFPDQLLSYNCSPSFNWSAHLEADEIAKFQKELGAMGFKFQFITLAGFHSLNYGMFDLAYGYAREGMTSFVDLQNREFKAAEERGFTAVKHQREVGAGYFDQIATTVDPNSSTTALKGSTEEGQFHN

SEQ ID NO: 62 - protein, 434 aa, Escherichia coli
MKTRTQQIEELQKEWTQPRWEGITRPYSAEDVVKLRGSVNPECTLAQLGAAKMWRLLHGESKKGYINSLGALTGGQALQQAKAGIEAVYLSGWQVAADANLAASMYPDQSLYPANSVPAVVERINNTFRRADQIQWSAGIEPGDPRYVDYFLPIVADAEAGFGGVLNAFELMKAMIEAGAAAVHFEDQLASVKKCGHMGGKVLVPTQEAIQKLVAARLAADVTGVPTLLVARTDADAADLITSDCDPYDSEFITGERTSEGFFRTHAGIEQAISRGLAYAPYADLVWCETSTPDLELARRFAQAIHAKYPGKLLAYNCSPSFNWQKNLDDKTIASFQQQLSDMGYKFQFITLAGIHSMWFNMFDLANAYAQGEGMKHYVEKVQQPEFAAAKDGYTFVSHQQEVGTGYFDKVTTIIQGGTSSVTALTGSTEESQF

SEQ ID NO: 63 - protein, 394 aa, unidentified; identical to SEQ ID NO: 3

SEQ ID NO: 64 - protein, 330 aa, unidentified
MVKAAVLGASGGIGQPLSLLLKTCPLVEELALYDVVNTPGVAADLSHISSIAKISGFLPKDDGLKQALTGANIVVIPAGIPRKPGMTRDDLFKINAGIVRDLVKGIAEFCPKAFVLVISNPVNSTVPIAAEVLKAAGVFDPKRLFGVTTLDVVRAETFTQEFSGQKDPSAVQIPVVGGHSGETIVPLFSKTTPAIQIPEEKYDALIHRVQFGGDEVVQAKDGAGSATLSMAYAGYRFAESVIKASKGQTGIVEPTFVYLPGIPGGDEIVKATGVEFFSTLVTLGTNGAEKASNVLEGVTEKEKKLLEACTKGLKGNIEKGIDFVKNPPPK

SEQ ID NO: 65 - protein, 600 aa, unidentified
MNNNNITPESDSMKSNDNDQTNDYMPDVADFDHTQTNTNEIARAISHPGSVLSRVASYVSRKDRYVDENGNEVWQDDEVSILMEEDETPDFTWKNIRHYAITRFTTLTELHRVSMENINPIPELRKMTLHNWNYFFMGYAAWLCAAWAFFAVSVSTAPLATLYGKETKDISWGLSLVLFVRSAGAIIFGIWTDNYSRKWPYITCLGLFLICQLCTPWAKTYTQFLGVRWISGIAMGGIYACASATAIEDAPVKARSFLSGLFFTAYAMGFIFAIIFYRAFLNVNGENYWKVQFWFSIWLPAVLILWRLVWPETKYFTKVLKARQLMRDDAIAKNGGQPLPKLSFKQKFANVKKTVSKYWLLFGYLILLLVGPNYLTHASQDLFPTMLRAQLRFSEDAVTVAIVVVCLGSIAGGMFFGQLMEITGRRVGLLLALIMAGCFTYPAFMLKTSSAVLGAGFMLWFSILGVWGVLPIHLSELSPPEARALVSGLAYQLGNLASAASVVIENDLADLYPIEWNSAVKVTNKDYSKVMAILTGSSVIFTFVLVFVGHEKFHRDLSSPHLKSYIERVDQTEEVAAMTGSTANSISSKPSDDQLEKVSV

SEQ ID NO: 66 - protein, 323 aa, Escherichia coli
MKPSVILYKTLPDDLLQHLEEHFSVTQVKNLRPETVSQHAEAFAQAEGLLGSSEKVDAALLEKMPKLRATSTVSVGYDNFDVEALNARRVLLMHTPTVLTETVADTVMALVLSTARRVVEVAERVKAGEWTKSIGPDWFGTDVHHKTLGIVGMGRIGMALAQRAHFGFGMPILYNARRQHPQAEERFNARYCDLDTLLQEADFVCLILPLSEETHHLFGQAQFAKMKSSAIFINAGRGPVVDEQALIAALQNGEIHAAGLDVFEHEPLAKDSPLLSLPNVVALPHIGSATHETRYNMAACAVDNLIDALNGNVEKNCVNPQVK

SEQ ID NO: 67 - protein, 380 aa, unidentified
MLTPPKFEDEKQLGPVGIRERLRHFTWAWYTLTMSGGGLAVLIISQPFGFRGLREIGIAVYILNLILFALVCSTMAIRFILHGNLLESLRHDREGLFFPTFWLSVATIICGLSRYFGEESNESFQLALEALFWIYCVCTLLVAIIQYSFVFSSHKYGLQTMMPSWILPAFPIMLSGTIASVIGEQQPARAALPIIGAGVTFQGLGFSISFMMYAHYIGRLMESGLPHSDHRPGMFICVGPPAFTALALVGMSKGLPEDFKLLHDAHALEDGRIIELLAISAGVFLWALSLWFFCIAIVAVIRSPPEAFHLNWWAMVFPNTGFTLATITLGKALNSNGVKGVGSAMSICIVCMYIFVFVNNVRAVIRKDIMYPGKDEDVSD

SEQ ID NO: 68 - protein, 550 aa, unidentified
MAAPFRQPEEAVDDTEFIDDHHEHLRDTVHHRLRANSSIMHFQKILVANRGEIPIRIFRTAHELSLQTVAIYSHEDRLSMHRQKADEAYMIGHRGQYTPVGAYLAGDEIIKIALEHGVQLIHPGYGFLSENADFARKVENAGIVFVGPTPDTIDSLGDKVSARRLAIKCEVPVVPGTEGPVERYEEVKAFTDTYGFPIIIKAAFGGGGRGMRVVRDQAELRDSFERATSEARSAFGNGTVFVERFLDKPKHIEVQLLGDSHGNVVHLFERDCSVQRRHQKVVEVAPAKDLPADVRDRILADAVKLAKSVNYRNAGTAEFLVDQQNRHYFIEINPRIQVEHTITEEITGIDIVAAQIQIAAGASLEQLGLTQDRISARGFAIQCRITTEDPAKGFSPDTGKIEVYRSAGGNGVRLDGGNGFAGAIITPHYDSMLVKCTCRGSTYEIARRKVVRALVEFRIRGVKTNIPFLTSLLSHPTFVDGNCWTTFIDDTPELFSLVGSQNRAQKLLAYLGDVAVNGSSIKGQIGEPKLKGDVIKPKLFDAEGKPLDVS

SEQ ID NO: 69 - protein, 2386 aa, unidentified
MAAPFRQPEEAVDDTEFIDDHHEHLRDTVHHRLRANSSIMHFQKILVANRGEIPIRIFRTAHELSLQTVAIYSHEDRLSMHRQKADEAYMIGHRGQYTPVGAYLAGDEIIKIALEHGVQLIHPGYGFLSENADFARKVENAGIVFVGPTPDTIDSLGDKVSARRLAIKCEVPVVPGTEGPVERYEEVKAFTDTYGFPIIIKAAFGGGGRGMRVVRDQAELRDSFERATSEARSAFGNGTVFVERFLDKPKHIEVQLLGDSHGNVVHLFERDCSVQRRHQKVVEVAPAKDLPADVRDRILADAVKLAKSVNYRNAGTAEFLVDQQNRHYFIEINPRIQVEHTITEEITGIDIVAAQIQIAAGASLEQLGLTQDRISARGFAIQCRITTEDPAKGFSPDTGKIEVYRSAGGNGVRLDGGNGFAGAIITPHYDSMLVKCTCRGSTYEIARRKVVRALVEFRIRGVKTNIPFLTSLLSHPTFVDGNCWTTFIDDTPELFSLVGSQNRAQKLLAYLGDVAVNGSSIKGQIGEPKLKGDVIKPKLFDAEGKPLDVSAPCTKGWKQILDREGPAAFAKAVRANKGCLIMDTTWRDAHQSLLATRVRTIDLLNIAHETSYAYSNAYSLECWGGATFDVAMRFLYEDPWDRLRKMRKAVPNIPFQMLLRGANGVAYSSLPDNAIYHFCKQAKKCGVDIFRVFDALNDVDQLEVGIKAVHAAEGVVEATMCYSGDMLNPHKKYNLEYYMALVDKIVAMKPHILGIKDMAGVLKPQAARLLVGSIRQRYPDLPIHVHTHDSAGTGVASMIACAQAGADAVDAATDSMSGMTSQPSIGAILASLEGTEQDPGLNLAHVRAIDSYWAQLRLLYSPFEAGLTGPDPEVYEHEIPGGQLTNLIFQASQLGLGQQWAETKKAYEAANDLLGDIVKVTPTSKVVGDLAQFMVSNKLTPEDVVERAGELDFPGSVLEFLEGLMGQPFGGFPEPLRSRALRDRRKLEKRPGLYLEPLDLAKIKSQIREKFGAATEYDVASYAMYPKVFEDYKKFVQKFGDLSVLPTRYFLAKPEIGEEFHVELEKGKVLILKLLAIGPLSEQTGQREVFYEVNGEVRQVAVDDNKASVDNTSRPKADVGDSSQVGAPMSGVVVEIRVHDGLEVKKGDPLAVLSAMKMEMVISAPHSGKVSSLLVKEGDSVDGQDLVCKIVKAMAAPFRQPEEAVDDTEFIDDHHEHLRDTVHHRLRANSSIMHFQKILVANRGEIPIRIFRTAHELSLQTVAIYSHEDRLSMHRQKADEAYMIGHRGQYTPVGAYLAGDEIIKIALEHGVQLIHPGYGFLSENADFARKVENAGIVFVGPTPDTIDSLGDKVSARRLAIKCEVPVVPGTEGPVERYEEVKAFTDTYGFPIIIKAAFGGGGRGMRVVRDQAELRDSFERATSEARSAFGNGTVFVERFLDKPKHIEVQLLGDSHGNVVHLFERDCSVQRRHQKVVEVAPAKDLPADVRDRILADAVKLAKSVNYRNAGTAEFLVDQQNRHYFIEINPRIQVEHTITEEITGIDIVAAQIQIAAGASLEQLGLTQDRISARGFAIQCRITTEDPAKGFSPDTGKIEVYRSAGGNGVRLDGGNGFAGAIITPHYDSMLVKCTCRGSTYEIARRKVVRALVEFRIRGVKTNIPFLTSLLSHPTFVDGNCWTTFIDDTPELFSLVGSQNRAQKLLAYLGDVAVNGSSIKGQIGEPKLKGDVIKPKLFDAEGKPLDVSAPCTKGWKQILDREGPAAFAKAVRANKGCLIMDTTWRDAHQSLLATRVRTIDLLNIAHETSYAYSNAYSLECWGGATFDVAMRFLYEDPWDRLRKMRKAVPNIPFQMLLRGANGVAYSSLPDNAIYHFCKQAKKCGVDIFRVFDALNDVDQLEVGIKAVHAAEGVVEATMCYSGDMLNPHKKYNLEYYMALVDKIVAMKPHILGIKDMAGVLKPQAARLLVGSIRQRYPDLPIHVHTHDSAGTGVASMIACAQAGADAVDAATDSMSGMTSQPSIGAILASLEGTEQDPGLNLAHVRAIDSYWAQLRLLYSPFEAGLTGPDPEVYEHEIPGGQLTNLIFQASQLGLGQQWAETKKAYEAANDLLGDIVKVTPTSKVVGDLAQFMVSNKLTPEDVVERAGELDFPGSVLEFLEGLMGQPFGGFPEPLRSRALRDRRKLEKRPGLYLEPLDLAKIKSQIREKFGAATEYDVASYAMYPKVFEDYKKFVQKFGDLSVLPTRYFLAKPEIGEEFHVELEKGKVLILKLLAIGPLSEQTGQREVFYEVNGEVRQVAVDDNKASVDNTSRPKADVGDSSQVGAPMSGVVVEIRVHDGLEVKKGDPLAVLSAMKMEMVISAPHSGKVSSLLVKEGDSVDGQDLVCKIVKA

SEQ ID NO: 70 - protein, 330 aa, unidentified; identical to SEQ ID NO: 64

SEQ ID NO: 71 - protein, 330 aa, unidentified; identical to SEQ ID NO: 64

SEQ ID NO: 72 - protein, 568 aa, unidentified
MTKPAELYCTSLEGVQVVGEVSDKPLLSEATPRDILTIDALKFVVLLHRAFNGTRKQLLANRQDVQKRLDAGESLTFLKETEYIRNDPNWRCATNHPKLMCRKVEITGPPDAKMIINAFNTNVHTYMTDFEDSCAPTWSNMLYGQVNLYDAIRDKIDFTNDKTGKRYKINREGRRVPVMIVRPRGWHMVDQHILVDGEPISASILDFGLFFYHNAKYLISQGLGPFFYLPKMEHWKEAKLWNDIFCVAQDCLEIPRGTIKATVLIETLPISYQLDEVLYALKDHSSGLNCGRWDYMFSTIKRLRNQKDKILPDRQQVTMTVPFMTNYVKQLIKVCHKRGVHAMGGMAAFIPIKNDEEANKVALEAVRKDKLREVLAGHDGTWIAHPGLLDTALSVFEEHMPTPNQLYKLKDDVSVTESDLVDTNIEGGQITMKGLNANIYIGLNYMESWLRGLGCVPINHLMEDAATAEVSRLQLWSWCKHQVKMNDTGKTITPDFVCQLIDEEVARCSHKEGNKYELAAQCLKDEISGKKPVAEFLTDILYPYIATPGKPVDLNSLKQSTPVCASRL

SEQ ID NO: 73 - protein, 554 aa, Saccharomyces cerevisiae
MVKVSLDNVKLLVDVDKEPFFKPSSTTVGDILTKDALEFIVLLHRTFNNKRKQLLENRQVVQKKLDSGSYHLDFLPETANIRNDPTWQGPILAPGLINRSTEITGPPLRNMLINALNAPVNTYMTDFEDSASPTWNNMVYGQVNLYDAIRNQIDFDTPRKSYKLNGNVANLPTIIVRPRGWHMVEKHLYVDDEPISASIFDFGLYFYHNAKELIKLGKGPYFYLPKMEHHLEAKLWNDVFCVAQDYIGIPRGTIRATVLIETLPAAFQMEEIIYQLRQHSSGLNCGRWDYIFSTIKRLRNDPNHILPNRNQVTMTSPFMDAYVKRLINTCHRRGVHAMGGMAAQIPIKDDPAANEKAMTKVRNDKIRELTNGHDGSWVAHPALAPICNEVFINMGTPNQIYFIPENVVTAANLLETKIPNGEITTEGIVQNLDIGLQYMEAWLRGSGCVPINNLMEDAATAEVSRCQLYQWVKHGVTLKDTGEKVTPELTEKILKEQVERLSKASPLGDKNKFALAAKYFLPEIRGEKFSEFLTTLLYDEIVSTKATPTDLSKL

SEQ ID NO: 74 - protein, 554 aa, Saccharomyces cerevisiae
MVKISLDNTALYADIDTTPQFEPSKTTVADILTKDALEFIVLLHRTFNSTRKQLLANRSNLQSKLDSGEYRFDFLPETEQIRNDPTWQGAIPAPGLINRSSEITGPPLRNMLVNALNAEVTTYMTDFEDSSSPTWENMIYGQVNLYDAIRNQIDFKTPRKEYRLKDDISRLPTLIVRPRGWHMVEKHLYIDDEPISASIFDFGLYFYHNAKELVKIGKGPYFYLPKMEHHMEVKLWNDIFCVAQDFIGMPRGTIRATVLIETLPAAFQMEEIIYQIREHSSGLNCGRWDYIFSTIKKLRNLPEHVLPNRDLVTMTSPFMDAYVKRLINTCHRRGVHAMGGMAAQIPIKDDPKANEAAMNKVRNDKIREMKNGHDGSWVAHPALAPICNEVFSNMGTANQIYFVPDVHVTSSDLLNTKIQDAQVTTEGIRVNLDIGLQYMEAWLRGSGCVPINHLMEDAATAEVSRCQLYQWVKHGVVLSDTGDKVTPELTAKILNEETAKLASASPLGEKNKFALAAKYFLPEVTGKIFSDFLTTLLYDEIIKPSAKPVDLSKL

SEQ ID NO: 75 - protein, 1034 aa, Saccharomyces cerevisiae
MLRTRVTALLCRATVRSSTNYVSLARTRSFHSQSILLKTAATDITSTQYSRIFNPDLKNIDRPLDTFARRHLGPSPSDVKKMLKTMGYSDLNAFIEELVPPNILKRRPLKLEAPSKGFCEQEMLQHLEKIANKNHYKVKNFIGKGYYGTILPPVIQRNLLESPEWYTSYTPYQPEISQGRLEALLNFQTVVSDLTGLPVANASLLDEGTAAGEAMLLSFNISRKKKLKYVIDKKLHQQTKSVLHTRAKPFNIEIIEVDCSDIKKAVDVLKNPDVSGCLVQYPATDGSILPPDSMKQLSDALHSHKSLLSVASDLMALTLLKPPAHYGADIVLGSSQRFGVPMGYGGPHAAFFAVIDKLNRKIPGRIVGISKDRLGKTALRLALQTREQHIKRDKATSNICTAQALLANVASSYCVYHGPKGLQNISRRIFSLTSILANAIENDSCPHELINKTWFDTLTIKLGNGISSEQLLDKALKEFNINLFAVDTTTISLALDETTTKADVENLLKVFDIENSSQFLSEDYSNSFPREFQRTDEILRNEVFHMHHSETAMLRYLHRLQSRDLSLANSMIPLGSCTMKLNSTVEMMPITWPQFSNIHPFQPSNQVQGYKELITSLEKDLCSITGFDGISLQPNSGAQGEYTGLRVIRSYLESKGENHRNVCLIPVSAHGTNPASAAMAGLKVVPVNCLQDGSLDLVDLKNKAEQHSKELAAVMITYPSTYGLFEPGIQHAIDIVHSFGGQVYLDGANMNAQVGLTSPGDLGADVCHLNLHKTFSIPHGGGGPAGAPICVKSHLIPHLPKHDVVDMITGIGGSKSIDSVSSAPYGNALVLPISYAYIKMMGNEGLPFSSVIAMLNSNYMMTRLKDHYKILFVNEMSTLKHCAHEFIVDLREYKAKGVEAIDVAKRLQDYGFHAPTLAFPVPGTLMIEPTESENLEELDRFCDAMISIKEEINALVAGQPKGQILKNAPHSLEDLITSSNWDTRGYTREEAAYPLPFLRYNKFWPTVARLDDTYGDMNLICTCPSVEEIANETE

SEQ ID NO: 76 - protein, 616 aa, Saccharomyces cerevisiae
MSSSITDEKISGEQQQPAGRKLYYNTSTFAEPPLVDGEGNPINYEPEVYNPDHEKLYHNPSLPAQSIQDTRDDELLERVYSQDQGVEYEEDEEDKPNLSAASIKSYALTRFTSLLHIHEFSWENVNPIPELRKMTWQNWNYFFMGYFAWLSAAWAFFCVSVSVAPLAELYDRPTKDITWGLGLVLFVRSAGAVIFGLWTDKSSRKWPYITCLFLFVIAQLCTPWCDTYEKFLGVRWITGIAMGGIYGCASATAIEDAPVKARSFLSGLFFSAYAMGFIFAIIFYRAFGYFRDDGWKILFWFSIFLPILLIFWRLLWPETKYFTKVLKARKLILSDAVKANGGEPLPKANFKQKMVSMKRTVQKYWLLFAYLVVLLVGPNYLTHASQDLLPTMLRAQLGLSKDAVTVIVVVTNIGAICGGMIFGQFMEVTGRRLGLLIACTMGGCFTYPAFMLRSEKAILGAGFMLYFCVFGVWGILPIHLAELAPADARALVAGLSYQLGNLASAAASTIETQLADRYPLERDASGAVIKEDYAKVMAILTGSVFIFTFACVFVGHEKFHRDLSSPVMKKYINQVEEYEADGLSISDIVEQKTECASVKMIDSNVSKTYEEHIETV

SEQ ID NO: 77 - protein, 604 aa, unidentified
MSEGNEEKGQGQGQGQDHGHFPEGRTFLHQVESKRSVHTHHSVPMNSSDSQSNASDPEELAPFPSVVDESELDESELEEIQNEDVSIIYKDDEEKPDLSWASICRYAGTRITSLFQIHKVSMDNINPFPELRKMTLSNWNYFFMGYLAWLCAAWAFFAVSVSTAPLAKLYDKPTKDISWGLSLVLFVRSAGAIIFGLWTDNYSRKWPYITCLGLFLICQLCTPWAKTYTQFLGVRWISGIAMGGIYGCASATAIEDAPVKARSILSGLFFSAYAMGFIFAIIFYRAFLHVNGENYWKVQFWFSIFLPALLMLWRLVWPETKYFTKVLKARELMKQDAIASNGGNPIPKTTFKEKMANTKRIICKYWLLFGYLVLLLVGPNYLTHGSQDLFPTMIRSQLNFSEDAVTVAIVVVNLGGICGGLFFGQLMEVTGRRLGLLIALTMAGCFTYPAFMLKTSSAVLGAGFMLFFSVFGVWGVIPIHLSELSPPEARALVAGLAYQLGNLASAASVVIENDLADIYPLARDAAGKVIKKDYAKVMAILTGSVVIFTFVMVLIGHEKFHRDLSSAVLKAYMEKVDRNEEDARLGVSSLSISSKSKEEQVENA

SEQ ID NO: 78 - protein, 614 aa, unidentified
MSSITDGKTSNEQQAAGRKLYYNTSTFAEPPLVDEEGNPINYEPEVYNPDHEKLYHNPSLPAVSIQDTRDDELLERVYSQDQGVEYEEDEEDKPNLSLASVKSYAKTRFTSLSHVHEFSWQNINPVPELRKMTWQNWNYFFMGYFAWLAAAWAFFCVSVSVAPLAELYGRPTKDITWGLGLVLFVRSAGAVIFGLWTDKSSRKWPYIACLFLFVIAQLCTPWCDTYEKFLGVRWITGIAMGGIYGCASATAIEDAPVKARSFLSGLFFSAYAMGFIFAIIFYRAFGYFREDGWKILFWFSIFLPILLIFWRLLWPETRYFTKVLKARKLILSDAVKANGGEPLPKANFRQKMVSMKRTVRKYWLLFTYLVILLVGPNYLTHASQDLLPTMLRAQLGLSKDAVTVIVVVTNIGAICGGMIFGQFMEVTGRRVGLLIACTMGGCFTYPAFMLRTEKAILGAGFMLYFCVFGVWGILPIHLAELAPADARALVAGLAYQLGNLASAAASTIETELAERYPLERDASGTVVKEDYAKVMAILTGSVFIFTFGCVFVGHEKFHRDLSSPVMKKYINQVDEYEADGISISDIVEQKTECASVKMIDSNTSKTYEEHVETV

SEQ ID NO: 79 - protein, 541 aa, Candida albicans
MEEKARADHISITNSNELNYDDYSNSYKNQPELDDELFNEKPDLSLSSISRYFATRLTTLLDLPLYHTHKKWYEVINPIPGLKSMSKSDWNFYCLGFFAWALDAMDFFCVSVAAPEIANTLNISVTDVTWGVTLVLMLRSVGAVIFGIASDYFGRKWTYISIVTLFVVVEVGTGFVQTYQQFLGVRAIFGILMGAMYPIAMVTALEGQPIAARSVLSGLFLPGYCFGYIMAMVWYRAFAGTYKEGEGWRSLIWFSGGLSLILIVWRLFTPESPDYIKMKIKKEKFNQQQRLKEQEQNGGVAVKEKKFWQKIDKSILVTFKTEWLIFSYLVLLYAGWNFTTHGSQDLYVTMITKQYHVGLDKKTVIIVVSNIGGIIGGIIMGQASELLGRRLTVVISIVCAGAFLYPSFFNPDRNWPAYIFLNAFVFGSFSVGPAYLLELVNSTHRTLLSGVAYQLGNLVSSASATIEAKIGERFPLKDQPGMFDYGKVMCIFCGAIFAYMIFIIFLGPERFHRDLQIHDTDEIEELQQDSSSNNESVKERV

SEQ ID NO: 80 - protein, 324 aa, Escherichia coli
MKPSVILYKALPDDLLQRLQEHFTVHQVANLSPQTVEQNAAIFAEAEGLLGSNENVDAALLEKMPKLRATSTISVGYDNFDVDALTARKILLMHTPTVLTETVADTLMALVLSTARRVVEVAERVKAGEWTASIGPDWYGTDVHHKTLGIVGMGRIGMALAQRAHFGFNMPILYNARRHHKEAEERFNARYCDLDTLLQESDFVCLILPLTDETHHLFGAEQFAKMKSSAIFINAGRGPVVDENALIAALQKGEIHAAGLDVFEQEPLSVDSPLLSMANVVAVPHIGSATHETRYGMAACAVDNLIDALQGKVEKNCVNPHVAD

SEQ ID NO: 81 - protein, 324 aa, Escherichia fergusonii
MKPSIILYKKLPDVLLQRLEEHFTVTQVSNLSPQTVEQHAEAFANAEGLLGSSEKVDDALLEKMPKLRATSTISVGYDNFDVDALNARKVLLMHTPTVLTETVADTLMALILATSRRVVEVAERVKAGEWTASIGPDWFGSDVHHKTLGIVGMGRIGMALAQRAHFGFNMPILYNARRQHPQAEERFNARYCDLDTLLQEADFVCLILPLTEETHHLFGAEQFAKMKSSAIFINAGRGPVVDEKALISALQKGEIHAAGLDVFEREPLPVDSPLLAMPNVVALPHIGSATHETRYNMAACAVDNLIAALQGNVDKNCVNPQVAK

SEQ ID NO: 82 - protein, 324 aa, Kluyvera georgiana
MKPSIILYKALPDDLLRRLEEHFSVTQVPNLRPETVAQHAQAFANAEGLLGSSETVNTALLEKMPKLRATSTISVGYDNFDVEALNARRVLLMHTPTVLTETVADTVMALVLSSARRVVEVAERVKGGEWTKSIGPDWFGIDVHHKTLGIVGMGRIGMALAQRAHFGFNMPILYNARRRHEQAEERFNARYCDLNTLLDEADFVCLILPLTDETYHLFGAEQFARMKSSAIFINAGRGPVVDEKALISALRNGDIHAAGLDVFEQEPLPASSPLLKMPNVVALPHIGSATHETRYNMAACAVDNLIDALQGKVEKNCVNPQVAG

SEQ ID NO: 83 - protein, 324 aa, Citrobacter koseri
MKPSIILYKALPDDLLHRLEEHFTVTQVPNLRPETVEQHAQAFASAEGLLGSSETVNSALLEKMPKLRAASTVSVGYDNFDVAALNARSVLLMHTPTVLTETVADTVMALVLSTARRVVEVAERVKVGEWTKSIGPDWFGTDVHHKTLGIVGMGRIGLALAQRAHFGFNMPILYNARRHHPEAEERFNARYCDLDTLLQAADFVCLILPLTEETHHLFGAAQFAKMKSSAIFINAGRGPVVDETALIAALQSGEIHAAGLDVFEQEPLPVDSPLLSLPNVVALPHIGSATHETRYNMAACAVDNLIDALQGKVEKNCVNPQVAG

SEQ ID NO: 84 - protein, 324 aa, Salmonella sp.
MKPSIILYKALPDDLLHRLEEHFTVTQVPNLRPETVEQHTQAFASAEGLLGSSETVNSALLEKMPKLRAASTVSVGYDNFDVAALNARSVLLMHTPTVLTETVADTVMALVLSTARRVVEVAERVKVGEWTKSIGPDWFGTDVHHKTLGIVGMGRIGLALAQRAHFGFNMPILYNARRHHPEAEERFNARYCDLDTLLQTADFVCLILPLTEETHHLFGAAQFAKMKSSAIFINAGRGPVVDENALIAALQSGEIHAAGLDVFEQEPLPVDSPLLSLPNVVALPHIGSATHETRYNMAACAVDNLIDALQGKVEKNCVNPQVAG